SEQUENCE LISTING (109 sequences)

SEQ ID NO: 1 - protein, 41 aa, Bacillus anthracis
MSNNNYSNGLNPDESLSASAFDPNLVGPTLPPIPPFTLPTG

SEQ ID NO: 2 - protein, 332 aa, Bacillus anthracis
MSNNNYSNGLNPDESLSASAFDPNLVGPTLPPIPPFTLPTGPTGPFTTGPTGPTGPTGPTGPTGPTGPTGPTGDTGTTGPTGPTGPTGPTGPTGPTGPTGPTGPTGFTPTGPTGPTGPTGDTGTTGPTGPTGPTGPTGPTGDTGTTGPTGPTGPTGPTGPTGPTGPTFTGPTGPTGPTGATGLTGPTGPTGPSGLGLPAGLYAFNSGGISLDLGINDPVPFNTVGSQFFTGTAISQLDADTFVISETGFYKITVIANTATASVLGGLTIQVNGVPVPGTGSSLISLGAPFTIVIQAITQITTTPSLVEVIVTGLGLSLALGTSASIIIEKVA

SEQ ID NO: 3 - protein, 33 aa, Bacillus anthracis
MSEKYIILHGTALEPNLIGPTLPPIPPFTFPNG

SEQ ID NO: 4 - protein, 209 aa, Bacillus anthracis
MSEKYIILHGTALEPNLIGPTLPPIPPFTFPNGPTGITGPTGATGFTGIGITGPTGVTGPTGIGITGPTGATGLGILPVFGTITTDVGIGFSVIVNTNINFTLPGPVSGTTLNPVDNSIIINTTGVYSVSFSIVFVIQAISSSILNLTINDSIQFAIESRIGGGPGVRATSARTDLLSLNQGDVLRVRIREATGDIIYSNASLVVSKVD

SEQ ID NO: 5 - protein, 44 aa, Bacillus anthracis
MVKVVEGNGGKSKIKSPLNSNFKILSDLVGPTFPPVPTGMTGIT

SEQ ID NO: 6 - protein, 647 aa, Bacillus anthracis
VVKVVEGNGGKSKIKSPLNSNFKILSDLVGPTFPPVPTGMTGITGSTGATGNTGPTGETGATGSAGITGSTGPTGNTGGTGSTGPTGNTGATGSTGVTGSTGVTGSTGVTGSTGVTGSTGPTGETGGTGSTGVTGSTGATGSTGVTGNTGPTGSTGATGNTGSIGETGGTGSMGPTGETGVTGSTGGTGSTGVTGNTGPTGSTGVTGSTGVTGSTGPTGSTGVTGSTGPTGSTGVTGSTGVTGNMGPTGSTGVTGNTGSTGTTGATGETGPMGSTGATGTTGPTGETGETGETGGTGSTGPTGNTGATGSTGVTGSTGVTGSTGVTGETGPTGSTGATGNTGPTGETGGTGSTGATGSTGVTGNTGPTGSTGVTGNTGATGETGPTGNTGATGNTGPTGETGVTGSTGPTGETGVTGSTGPTGNTGATGETGATGSTGVTGNTGSTGETGPTGSTGPTGSTGATGVTGNTGPTGSTGATGATGSTGPTGSTGTTGNTGVTGDTGPTGATGVSTTATYAFANNTSGSVISVLLGGTNIPLPNNQNIGPGITVSGGNTVFTVANAGNYYIAYTINLTAGLLVSSRITVNGSPLAGTINSPTVATGSFSATIIASLPAGAAVSLQLFGVVALATLSTATPGATLTIIRLS

SEQ ID NO: 7 - protein, 34 aa, Bacillus anthracis
MKQNDKLWLDKGIIGPENIGPTFPVLPPIHIPTG

SEQ ID NO: 8 - protein, 366 aa, Bacillus anthracis
MKQNDKLWLDKGIIGPENIGPTFPVLPPIHIPTGITGATGATGITGATGPTGTTGATGATGITGVTGATGITGVTGATGITGVTGATGITGVTGPTGITGATGPTGITGATGPAGITGVTGPTGITGATGPTGTTGVTGPTGDTGLAGATGPTGATGLAGATGPTGDTGATGPTGATGLAGATGPTGATGLTGATGATGATGGGAIIPFASGTTPALLVNAVLANTGTLLGFGFSQPGIAPGVGGTLTILPGVVGDYAFVAPRDGIITSLAGFFSATAALAPLTPVQIQMQIFIAPAASNTFTPVAPPLLLTPALPAIAIGTTATGIQAYNVPVVAGDKILVYVSLTGASPIAAVAGFVSAGLNIV

SEQ ID NO: 9 - protein, 30 aa, Bacillus anthracis
MDEFLSSAALNPGSVGPTLPPMQPFQFRTG

SEQ ID NO: 10 - protein, 77 aa, Bacillus anthracis
MDEFLSSAALNPGSVGPTLPPMQPFQFRTGPTGSTGAKGAIGNTEPYWHTGPPGIVLLTYDFKSLIISFAFRILPIS

SEQ ID NO: 11 - protein, 39 aa, Bacillus weihenstephanensis
  source 1..39: note = Bacillus weihenstephensis
MFDKNEIQKINGILQANALNPNLIGPTLPPIPPFTLPTG

SEQ ID NO: 12 - protein, 299 aa, Bacillus weihenstephanensis
  source 1..299: note = Bacillus weihenstephensis
MFDKNEIQKINGILQANALNPNLIGPTLPPIPPFTLPTGPTGVTGPTGVTGPTGVTGPTGVTGPTGVTGPTGVTGPTGVTGPTGVTGPTGVTGPTGVTGPTGVTGPTGVTGPTGVTGPTGVTGPTGETGPTGGTEGCLCDCCVLPMQSVLQQLIGETVILGTIADTPNTPPLFFLFTITSVNDFLVTVTDGTTTFVVNISDVTGVGFLPPGPPITLLPPTDVGCECECRERPIRQLLDAFIGSTVSLLASNGSIAADFSVEQTGLGIVLGTLPINPTTTVRFAISTCKITAVNITPITM

SEQ ID NO: 13 - protein, 39 aa, Bacillus weihenstephanensis
  source 1..39: note = Bacillus weihenstephensis
MFDKNEMKKTNEVLQANALDPNIIGPTLPPIPPFTLPTG

SEQ ID NO: 14 - protein, 289 aa, Bacillus weihenstephanensis
  source 1..289: note = Bacillus weihenstephensis
MFDKNEMKKTNEVLQANALDPNIIGPTLPPIPPFTLPTGPTGPTGPTGPTGPTGPTGPTGPTGPTGPTGPTGPTGPTGLTGPTGPTGLTGPTGLTGPTGPTGLTGQTGSTGPTGATEGCLCDCCVFPMQEVLRQLVGQTVILATIADAPNVAPRFFLFNITSVNDFLVTVTDPVSNTTFVVNISDVIGVGFSLTVPPLTLLPPADLGCECDCRERPIRELLDTLIGSTVNLLVSNGSIATGFNVEQTALGIVIGTLPIPINPPPPTLFRFAISTCKITAVDITPTPTAT

SEQ ID NO: 15 - protein, 49 aa, Bacillus cereus
MSRKDKFNRSRMSRKDRFNSPKIKSEISISPDLVGPTFPPIPSFTLPTG

SEQ ID NO: 16 - protein, 189 aa, Bacillus cereus
MSRKDKFNRSRMSRKDRFNSPKIKSEISISPDLVGPTFPPIPSFTLPTGITGPTFNINFRAEKNVAQSFTPPADIQVSYGNIIFNNGGGYSSVTNTFTAPINGIYLFSASIGFNPTLGTTSTLRITIRKNLVSVASQTGTITTGGTPQLEITTIIDLLASQTIDIQFSAAESGTLTVGSSNFFSGALLP

SEQ ID NO: 17 - protein, 33 aa, Bacillus cereus
MNEEYSILHGPALEPNLIGPTLPSIPPFTFPTG

SEQ ID NO: 18 - protein, 84 aa, Bacillus cereus
MNEEYSILHGPALEPNLIGPTLPSIPPFTFPTGPTGITGPTGATGFTGIGITGPTGVTGPTGIGITGPTGATGPTGIGITGPTG

SEQ ID NO: 19 - protein, 39 aa, Bacillus cereus
MKNRDNNRKQNSLSSNFRIPPELIGPTFPPVPTGFTGIG

SEQ ID NO: 20 - protein, 1056 aa, Bacillus cereus
MKNRDNNRKQNSLSSNFRIPPELIGPTFPPVPTGFTGIGITGPTGPQGPTGPQGPRGLQGPMGEMGPTGPQGVQGIQGSVGPIGATGPEGQQGPQGLRGPQGETGATGPGGVQGLQGPIGPTGATGAQGIQGIQGLQGPIGATGPEGSQGIQGVQGLPGATGPQGIQGAQGIQGTPGPSGNTGATGATGATGQGITGPTGITGPTGITGPSGGPPGPTGPTGATGPGGGPSGSTGATGATGNTGATGSTGVTGATGSTGPTGSTGAQGLQGIQGIQGPIGPTGPEGSQGIQGIPGPTGVTGEQGIQGVQGIQGATGATGDQGPQGIQGVIGPQGVTGATGDQGPQGIQGVPGPSGETGPQGVQGIQGPMGDIGPTGPEGPEGLQGPQGIQGVPGPVGATGPEGPQGIQGIQGPVGATGPQGPQGIQGIQGVQGITGATGVQGATGIQGIQGEIGATGPEGPQGVQGAQGAIGPTGPMGPQGVQGVQGIQGATGAQGVQGPQGIQGIQGPTGATGDMGATGATGEGTTGPTGVTGPTGVTGPSGGPAGPTGPTGPSGPAGVTGPSGGPPGPTGATGATGVTGDTGATGSTGVTGATGETGATGVTGLQGPQGIQGVQGEIGPTGPQGVQGPQGIQGVTGATGDQGPQGIQGPQGDIGPTGPQGIQGPQGSQGIQGATGGTGAQGPQGIQGPQGDIGLTGSQGPTGIQGIQGEIGPTGPEGPEGLQGPQGIQGIQGPVGATGPEGPQGIQGIQGVQGATGPQGPQGIQGIQGVQGITGATGAQGATGIQGIQGEIGATGPEGPQGVQGIQGAIGPTGPMGAQGVQGIQGIQGATGAQGVQGPQGIQGVQGPTGATGETGATGATGEGTTGPTGVTGPTGVTGPSGGPAGPTGPTGPSGPAGVTGPSGGPPGPTGATGATGVTGDTGATGSTGVTGATGATGATGVTGLQGPQGIQGVQGEIGPTGPQGIQGPQGIQGVTGATGAQGPQGIQGPQGDIGPTGSQGIQGPQGPQGIQGATGATGAQGPQGIQGPQGEIGPTGPQGPQGIQGPQGIQGPTG

SEQ ID NO: 21 - protein, 39 aa, Bacillus weihenstephanensis
  source 1..39: note = Bacillus weihenstephensis
MSDKHQMKKISEVLQAHALDPNLIGPPLPPITPFTFPTG

SEQ ID NO: 22 - protein, 365 aa, Bacillus weihenstephanensis
  source 1..365: note = Bacillus weihenstephensis
MSDKHQMKKISEVLQAHALDPNLIGPPLPPITPFTFPTGSTGPTGSTGSTGPTGSTGNTGPTGPTGPPVGTNLDTIYVTNDISNNVSAIDGNTNTVLTTIPVGTNPVGVGVNSSTNLIYVVNNGSDNISVINGSTNTVVATIPVGTQPFGVGVNPSTNLIYVANRTSNNVSVIKGGTNTVLTTIPVGTNPVGVGVNSSTNLIYVTNEIPNSVSVIKGGTNTVVATIPVGLFPFGVGVNSLTNLIYVVNNSPHNVSVIDGNTNTVLTTISVGTSPVGVGVNLSTNLIYVANEVPNNISVINGNTNTVLTTIPVGTTPFEVGVNSSTNLIYVSNLNSNNVSVINGSANTVIATVPVGSVPRGIGVKP

SEQ ID NO: 23 - protein, 30 aa, Bacillus weihenstephanensis
  source 1..30: note = Bacillus weihenstephensis
MDEFLSFAALNPGSIGPTLPPVPPFQFPTG

SEQ ID NO: 24 - protein, 160 aa, Bacillus weihenstephanensis
  source 1..160: note = Bacillus weihenstephensis
MDEFLSFAALNPGSIGPTLPPVPPFQFPTGPTGSTGSTGPTGSTGSTGPTGFNLPAGPASITLTSNETTACVSTQGNNTLFFSGQVLVNGSPTPGVVVSFSFSNPSLAFMVPLAVITNASGNFTAVFLAANGPGTVTVTASLLDSPGTMASVTITIVNCP

SEQ ID NO: 25 - protein, 30 aa, Bacillus weihenstephanensis
  source 1..30: note = Bacillus weihenstephensis
MDEFLSSTALNPCSIGPTLPPMQPFQFPTG

SEQ ID NO: 26 - protein, 69 aa, Bacillus weihenstephanensis
  source 1..69: note = Bacillus weihenstephensis
MDEFLSSTALNPCSIGPTLPPMQPFQFPTGPTGSTGTTGPTGSIGPTGNTGLTGNTGPTGITGPTGDTG

SEQ ID NO: 27 - protein, 36 aa, Bacillus weihenstephanensis
  source 1..36: note = Bacillus weihenstephensis
MKERDRQNSLNSNFRISPNLIGPTFPPVPTGFTGIG

SEQ ID NO: 28 - protein, 934 aa, Bacillus weihenstephanensis
  source 1..934: note = Bacillus weihenstephensis
MKERDRQNSLNSNFRISPNLIGPTFPPVPTGFTGIGITGPTGPQGPTGPQGPRGFQGPMGEMGPTGPQGVQGIQGPAGQMGATGPEGQQGPQGLRGPQGETGATGPQGVQGLQGPIGPTGATGAQGIQGIQGLQGPIGATGPEGPQGIQGVQGVPGATGSQGIQGAQGIQGPQGPSGNTGATGVTGQGISGPTGITGPTGITGPSGGPPGPTGATGATGPGGGPSGSTGATGATGNTGVTGSAGVTGNTGSTGSTGETGAQGLQGIQGVQGPIGPTGPEGPQGIQGIPGPTGVTGEQGIQGVQGIQGITGATGDQGPQGIQGAIGPQGITGATGDQGPQGIQGVPGPTGDTGSQGVQGIQGPMGDIGPTGPEGPEGLQGPQGIQGVPGPAGATGPEGPQGIQGIQGPIGVTGPEGPQGIQGIQGIQGITGATGAQGATGVQGVQGNIGATGPEGPQGVQGTQGDIGPTGPMGPQGVQGIQGIQGPTGAQGVQGPQGIQGIQGPTGVTGDTGTTGATGEGTTGATGVTGPSGVTGPSGGPAGPTGPTGPSGPTGLTGPSGGPPGPTGATGVTGGVGDTGATGSTGVTGATGVTGATGATGLQGPQGIQGVQGDIGPTGPQGVQGPQGIQGITGATGDQGPQGIQGPQGIQGPTGPQGIQGGQGPQGIQGATGATGAQGPQGIQGIQGVQGPTGPQGPTGIQGVQGEIGPTGPQGVQGLQGPQGPTGDTGPTGPQGPQGIQGPTGATGATGSQGIQGPTGATGATGSQGIQGPTGATGATGATGATGATGATGATGVTGVSTTATYSFANNTSGSAISVLLGGTNIPLPNNQNIGPGITVSGGNTVFTVTNAGNYYIAYTINITAALLVSSRITVNGSPLAGTINSPAVATGSFNATIISNLAAGSAISLQLFGLLAVATLSTTTPGATLTIIRLS

SEQ ID NO: 29 - protein, 39 aa, Bacillus mycoides
VFDKNEIQKINGILQANALNPNLIGPTLPPIPPFTLPTG

SEQ ID NO: 30 - protein, 287 aa, Bacillus mycoides
VFDKNEIQKINGILQANALNPNLIGPTLPPIPPFTLPTGPTGGTGPTGVTGPTGVTGPTGVTGPTGVTGPTGVTGPTGVTGPTGVTGPTGVTGPTGVTGPTGVTGPTGVTGPTGVTGPTGGTEGCLCDCCVLPMQSVLQQLIGETVILGTIADTPNTPPLFFLFTITSVNDFLVTVTDGTTTFVVNISDVTGVGFLPPGPPITLLPPTDVGCECECRERPIRQLLDAFIGSTVSLLASNGSIAADFSVEQTGLGIVLGTLPINPTTTVRFAISTCKITAVNITPITM

SEQ ID NO: 31 - protein, 30 aa, Bacillus mycoides
MDEFLYFAALNPGSIGPTLPPVQPFQFPTG

SEQ ID NO: 32 - protein, 190 aa, Bacillus mycoides
MDEFLYFAALNPGSIGPTLPPVQPFQFPTGPTGSTGATGSTGSTGSTGPTGSTGSTGSTGSTGPTGPTGPTGSTGPTGPTGFNLPAGPASITLTSNETTACVSTQGNNTLFFSGQVLVNGSPTPGVVVSFSFSNPSLAFMVPLAVITNASGNFTAVFLAANGPGTVTVTASLLDSPGTMASVTITIVNCP

SEQ ID NO: 33 - protein, 21 aa, Bacillus mycoides
MDSKNIGPTFPPLPSINFPTG

SEQ ID NO: 34 - protein, 335 aa, Bacillus mycoides
MDSKNIGPTFPPLPSINFPTGVTGETGATGETGATGATGETGATGETGETGATGATGATGATGETGATGATGATGAAGATGETGATGETGATGETGATGETGATGVTGETGATGETGAAGETGITGVTGPTGETGATGETGATGATGITGATGITGVAGATGETGAAGETGPTGATGAIGAIGATGATGITGVTGATGETGAAGATGITGVTGATGETGAAGATGITGATGITGVAGATGITGPTGIPGTIPTTNLLYFTFSDGEKLIYTNADGIAQYGTTQILSPSEVSYINLFINGILQPQPFYEVTAGQLTLLDDEPPSQGSSIILQFIIIN

SEQ ID NO: 35 - protein, 22 aa, Bacillus thuringiensis
MIGPENIGPTFPILPPIYIPTG

SEQ ID NO: 36 - protein, 234 aa, Bacillus thuringiensis
MIGPENIGPTFPILPPIYIPTGETGPTGITGATGETGPTGITGPTGITGATGETGSTGITGATGETGSTGITGPIGITGATGETGPIGITGATGETGPTGITGSTGITGLTGVTGLTGETGPIGITGPTGITGPTGVTGATGPTGGIGPITTTNLLYYTFADGEKLIYTDTDGIPQYGTTNILSPSEVSYINLFVNGILQPQPLYEVSTGKLTLLDTQPPSQGSSIILQFIIIN

SEQ ID NO: 37 - DNA, 23 bp, synthetic construct
  source 1..23: mol_type = other DNA; note = Primer
ggatccatggctgaacacaatcc

SEQ ID NO: 38 - DNA, 24 bp, synthetic construct
  source 1..24: mol_type = other DNA; note = Primer
ggatccttaattcgtattctggcc

SEQ ID NO: 39 - DNA, 21 bp, synthetic construct
  source 1..21: mol_type = other DNA; note = Primer
ggatccatgaaacggtcaatc

SEQ ID NO: 40 - DNA, 24 bp, synthetic construct
  source 1..24: mol_type = other DNA; note = Primer
ggatccttactaatttggttctgt

SEQ ID NO: 41 - DNA, 21 bp, synthetic construct
  source 1..21: mol_type = other DNA; note = Primer
ggatccatgctaccaaaagcc

SEQ ID NO: 42 - DNA, 24 bp, synthetic construct
  source 1..24: mol_type = other DNA; note = Primer
ggatccttagtccgcaggcgtagc

SEQ ID NO: 43 - protein, 35 aa, Bacillus cereus
MSNNNIPSPFFFNNFNPELIGPTFPPIPPLTLPTG

SEQ ID NO: 44 - protein, 222 aa, Bacillus cereus
MSNNNIPSPFFFNNFNPELIGPTFPPIPPLTLPTGPTGSTGATGATGPTGATGPTGATGPTGATGATGSTGATGPTGATGTFSSANASIVTPAPQTVNNLAPIQFTAPVLISKNVTFNGIDTFTIQIPGNYFFIGAVMTSNNQAGPVAVGVGFNGIPVPSLDGANYGTPTGQEVVCFGFSGQIPAGTTINLYNISDKTISIGGATAAGSSIVAARLSFFRIS

SEQ ID NO: 45 - protein, 41 aa, Bacillus cereus
MFSEKKRKDLIPDNFLSAPALDPNLIGPTFPPIPSFTLPTG

SEQ ID NO: 46 - protein, 293 aa, Bacillus cereus
MFSEKKRKDLIPDNFLSAPALDPNLIGPTFPPIPSFTLPTGSTGPTGPTGDTGPTGPTATICIRTDPDNGCSVAEGSGTVASGFASHAEACNTQAIGDCSHAEGQFATASGTASHAEGFQTTASGFASHTEGSGTTADANFSHTEGINTIVDVLHPGSHIMGKNGTTRSSFSWHLANGLAVGPSLNSAVIEGVTGNLYLDGVVISPNAADYAEMFETIDGNLIDVGYFVTLYGEKIRKANANDDYILGVVSATPAMIADASDLRWHNLFVRDEWGRTQYHEVVVPEKKMAMEE

SEQ ID NO: 47 - protein, 49 aa, Bacillus cereus
MTRKDKFNRSRISRRDRFNSPKIKSEILISPDLVGPTFPPIPSFTLPTG

SEQ ID NO: 48 - protein, 83 aa, Bacillus cereus
MTRKDKFNRSRISRRDRFNSPKIKSEILISPDLVGPTFPPIPSFTLPTGVTGPTGNTGPTGITGPTGDTGPTGDTGPTGITGP

SEQ ID NO: 49 - protein, 38 aa, Bacillus cereus
MSRKDRFNSPKIKSEISISPDLVGPTFPPIPSFTLPTG

SEQ ID NO: 50 - protein, 163 aa, Bacillus cereus
MSRKDRFNSPKIKSEISISPDLVGPTFPPIPSFTLPTGITGPTGNTGPTGDTGPTGPTFNINFRAEKNGAQSFTPPADIQVSYGNIIFNNGGGYSSVTNTFTAPINGIYLFSANIGFNPTLGTTSTLRITIRKNLVSVASQTIDIQFSAAESGTLTVGSSNFF

SEQ ID NO: 51 - protein, 39 aa, Bacillus cereus
MKERDNKGKQHSLNSNFRIPPELIGPTFPPVPTGFTGIG

SEQ ID NO: 52 - protein, 323 aa, Bacillus cereus
MKERDNKGKQHSLNSNFRIPPELIGPTFPPVPTGFTGIGITGPTGPQGPTGPQGPRGFQGPMGEMGPTGPQGVQGIQGPAGQMGATGPEGQQGPEGLRGPVGATGATGLQGVQGIQGPIGSTGATGAQGIQGIQGLQGPIGATGPEGPQGIQGVQGLPGATGPQGVQGVQGVIGPQGPSGSTGGTGATGQGVTGPTGITGSTGVTGPSGGPPGPTGPTGATGPGGGPSGSTGVTGSTGNTGATGSPGVTGATGPTGSTGATGIQGSQGIQGIQGIQGPLGPTGPEGPQGIQGIPGPTGITGEQGIQGVQGIQGITGATGDQGT

SEQ ID NO: 53 - protein, 39 aa, Bacillus cereus
MRERDNKRQQHSLNPNFRISPELIGPTFPPVPTGFTGIG

SEQ ID NO: 54 - protein, 436 aa, Bacillus cereus
MRERDNKRQQHSLNPNFRISPELIGPTFPPVPTGFTGIGITGPTGPQGPTGPQGPRGFQGPMGEMGPTGPQGVQGIQGPVGPIGATGPEGQQGPQGLRGPQGETGATGPGGVQGLQGPIGPTGATGAQGVQGIQGLQGPIGATGPEGPQGIQGVQGLPGATGSQGIQGVQGIQGPQGPSGNTGATGATGQGITGPTGITGPTGITGPSGGPPGPTGPTGATGPGGGPSGSTGATGATGNTGATGNTGITGATGSTGPTGSTGAQGLQGIQGIQGPIGPTGPEGPQGIQGIPGPTGVTGEQGIQGVQGIQGITGATGDQGPQGIQGVIGAQGVTGATGDQGPQGIQGVPGPSGATGPQGVQGIQGPMGDIGPTGPEGPEGLQGPQGIQGVPGPVGATGPEGPQGIQGIQGVQGATGPQGPQGIQGIQGVQGITGATG

SEQ ID NO: 55 - protein, 36 aa, Bacillus thuringiensis
MKNRDNKGKQQSNFRIPPELIGPTFPPVPTGFTGIG

SEQ ID NO: 56 - protein, 470 aa, Bacillus thuringiensis
MKNRDNKGKQQSNFRIPPELIGPTFPPVPTGFTGIGITGPTGPQGPTGPQGPRGFQGPMGEMGPTGPQGVQGIQGPVGPIGATGPEGQQGAQGLRGPQGETGATGPQGVQGLQGPIGPTGATGAQGIQGIQGLQGPIGATGPEGPQGIQGVQGLPGATGPQGIQGAQGIQGTQGPSGNTGATGATGQGLTGPTGITGPTGITGPSGGPPGPTGPTGATGPGGGPSGSTGATGATGDTGATGSTGVTGATGAQGPQGVQGIQGPTGATGATGATGPQGIQGPQGIQGPTGATGATGSQGPTGNTGPTGSQGIQGPTGPTGAGATGATGATGATGVSTTATYAFANNTSGSIISVLLGGTNIPLPNNQNIGPGITVSGGNTVFTVANAGNYYIAYTINLTAGLLVSSRITVNGSPLAGTINSPAVAAGSFSATIIANLPAGAAVSLQLFGVIALATLSTATPGATLTIIRLS

SEQ ID NO: 57 - protein, 136 aa, Bacillus mycoides
MKFSKKSTVDSSIVGKRVVSKVNILRFYDARSCQDKDVDGFVDVGELFTIFRKLNMEGSVQFKAHNSIGKTYYITINEVYVFVTVLLQYSTLIGGSYVFDKNEIQKINGILQANALNPNLIGPTLPPIPPFTLPTG

SEQ ID NO: 58 - protein, 384 aa, Bacillus mycoides
MKFSKKSTVDSSIVGKRVVSKVNILRFYDARSCQDKDVDGFVDVGELFTIFRKLNMEGSVQFKAHNSIGKTYYITINEVYVFVTVLLQYSTLIGGSYVFDKNEIQKINGILQANALNPNLIGPTLPPIPPFTLPTGPTGGTGPTGVTGPTGVTGPTGVTGPTGVTGPTGVTGPTGVTGPTGVTGPTGVTGPTGVTGPTGVTGPTGVTGPTGVTGPTGGTEGCLCDCCVLPMQSVLQQLIGETVILGTIADTPNTPPLFFLFTITSVNDFLVTVTDGTTTFVVNISDVTGVGFLPPGPPITLLPPTDVGCECECRERPIRQLLDAFIGSTVSLLASNGSIAADFSVEQTGLGIVLGTLPINPTTTVRFAISTCKITAVNITPITM

SEQ ID NO: 59 - protein, 196 aa, Bacillus anthracis
MSNNNYSNGLNPDESLSASAFDPNLVGPTLPPIPPFTLPTGPTGPFTTGPTGPTGPTGPTGPTGPTGPTGPTGDTGTTGPTGPTGPTGPTGPTGPTGPTGPTGPTGFTPTGPTGPTGPTGDTGTTGPTGPTGPTGPTGPTGDTGTTGPTGPTGPTGPTGPTGPTGPTFTGPTGPTGPTGATGLTGPTGPTGPSGLG

SEQ ID NO: 60 - protein, 17 aa, Bacillus anthracis
MAFDPNLVGPTLPPIPP

SEQ ID NO: 61 - protein, 17 aa, Bacillus anthracis
MALEPNLIGPTLPPIPP

SEQ ID NO: 62 - protein, 17 aa, Bacillus weihenstephanensis
  source 1..17: note = Bacillus weihenstephensis
MALNPNLIGPTLPPIPP

SEQ ID NO: 63 - protein, 17 aa, Bacillus weihenstephanensis
  source 1..17: note = Bacillus weihenstephensis
MALDPNIIGPTLPPIPP

SEQ ID NO: 64 - protein, 17 aa, Bacillus cereus
MALEPNLIGPTLPSIPP

SEQ ID NO: 65 - protein, 17 aa, Bacillus weihenstephanensis
  source 1..17: note = Bacillus weihenstephensis
MALDPNLIGPPLPPITP

SEQ ID NO: 66 - protein, 17 aa, Bacillus weihenstephanensis
  source 1..17: note = Bacillus weihenstephensis
MALNPGSIGPTLPPVPP

SEQ ID NO: 67 - protein, 17 aa, Bacillus weihenstephanensis
  source 1..17: note = Bacillus weihenstephensis
MALNPCSIGPTLPPMQP

SEQ ID NO: 68 - protein, 17 aa, Bacillus mycoides
MALNPGSIGPTLPPVQP

SEQ ID NO: 69 - protein, 17 aa, Bacillus anthracis
MALNPGSVGPTLPPMQP

SEQ ID NO: 70 - protein, 17 aa, Bacillus cereus
MALDPNLIGPTFPPIPS

SEQ ID NO: 71 - protein, 799 aa, Bacillus mycoides
MKRKTPFKVFSSLAITTMLGCTFALGTSVAYAETTSQSKGSISTTPIDNNLIQEERLAEALKERGTIDQSASKEETQKAVEQYIEKKKGDQPNKEILPDDPAKEASDFVKKVKEKKMEEKEKVKKSVENASSEQTPSQNKKQLNGKVPTSPAKQAPYNGAVRTDKVLVLLVEFSDYKHNNIEQSPGYMYANDFSREHYQKMLFGNEPFTLFDGSKVKTFKQYYEEQSGGSYTTDGYVTEWLTVPGKAADYGADGKTGHDNKGPKGARDLVKEALKAAAEKGLDLSQFDQFDRYDTNGDGNQNEPDGVIDHLMVIHAGVGQEAGGGKLGDDAIWSHRSKLAQDPVAIEGTKSKVSYWDGKVAAHDYTIEPEDGAVGVFAHEFGHDLGLPDEYDTNYTGAGSPVEAWSLMSGGSWTGRIAGTEPTSFSPQNKDFLQKNMDGNWAKIVEVDYDKIKRGVGFPTYIDQSVTKSNRPGLVRVNLPEKSVETIKTGFGKHAYYSTRGDDMHTTLETPLFDLTKAANAKFDYKANYELEAECDFIEVHAVTEDGTKTLIDKLGDKVVKGDQDTTEGKWIDKSYDLSQFKGKKVKLQFDYITDPALTYKGFAMDNVNVTVDGKVVFSDDAEGQAKMKLNGFVVSDGTEKKPHYYYLEWRNYAGSDEGLKVGRGPVYNTGLVVWYADDSFKDNWVGRHPGEGFLGVVDSHPEAVVGNLNGKPVYGNTGLQIADAAFSLDQTPAWNVNSFTRGQFNYPGLPGVATFDDSKVYSNTQIPDAGRKVPQLGLKFQVVGQADDKSAGAIWIRR

SEQ ID NO: 72 - protein, 152 aa, Bacillus anthracis
MSCNENKHHGSSHCVVDVVKFINELQDCSTTTCGSGCEIPFLGAHNTASVANTRPFILYTKAGAPFEAFAPSANLTSCRSPIFRVESVDDDSCAVLRVLSVVLGDSSPVPPTDDPICTFLAVPNARLVSTSTCITVDLSCFCAIQCLRDVTI

SEQ ID NO: 73 - protein, 167 aa, Bacillus anthracis
MFSSDCEFTKIDCEAKPASTLPAFGFAFNASAPQFASLFTPLLLPSVSPNPNITVPVINDTVSVGDGIRILRAGIYQISYTLTISLDNSPVAPEAGRFFLSLGTPANIIPGSGTAVRSNVIGTGEVDVSSGVILINLNPGDLIRIVPVELIGTVDIRAAALTVAQIS

SEQ ID NO: 74 - protein, 156 aa, Bacillus anthracis
MSCNCNEDHHHHDCDFNCVSNVVRFIHELQECATTTCGSGCEVPFLGAHNSASVANTRPFILYTKAGAPFEAFAPSANLTSCRSPIFRVESIDDDDCAVLRVLSVVLGDTSPVPPTDDPICTFLAVPNARLISTNTCLTVDLSCFCAIQCLRDVTI

SEQ ID NO: 75 - protein, 182 aa, Bacillus anthracis
MEVGGTSVKNKNKSSTVGKPLLYIAQVSLELAAPKTKRIILTNFENEDRKEESNRNENVVSSAVEEVIEQEEQQQEQEQEQEEQVEEKTEEEEQVQEQQEPVRTVPYNKSFKDMNNEEKIHFLLNRPHYIPKVRCRIKTATISYVGSIISYRNGIVAIMPPNSMRDIRLSIEEIKSIDMAGF

SEQ ID NO: 76 - protein, 174 aa, Bacillus anthracis
MKERSENMRSSSRKLTNFNCRAQAPSTLPALGFAFNATSPQFATLFTPLLLPSTGPNPNITVPVINDTISTGTGIRIQVAGIYQISYTLTISLDNVPVTPEAARFFLTLNSSTNIIAGSGTAVRSNIIGTGEVDVSSGVILINLNPGDLIQIVPVEVIGTVDIRSAALTVAQIR

SEQ ID NO: 77 - protein, 796 aa, Bacillus thuringiensis
MSKKPFKVLSSIALTAVLGLSFGAGTQSAYAETPVNKTATSPVDDHLIPEERLADALKKRGVIDSKASETETKKAVEKYVENKKGENPGKEAANGDQLTKDASDFLKKVKDAKADTKEKLNQPATGTPAATGPVKGGLNGKVPTSPAKQKDYNGEVRKDKVLVLLVEYADFKHNNIDKEPGYMYSNDFNKEHYEKMLFGNEPFTLDDGSKIETFKQYYEEQSGGSYTVDGTVTKWLTVPGKAADYGADAPGGGHDNKGPKGPRDLVKDALKAAVDSGIDLSEFDQFDQYDVNGDGNKNQPDGLIDHLMIIHAGVGQEAGGGKLGDDAIWSHRWTVGPKPFPIEGTQAKVPYWGGKMAAFDYTIEPEDGAVGVFAHEYGHDLGLPDEYDTQYSGQGEPIEAWSIMSGGSWAGKIAGTTPTSFSPQNKEFFQKTIGGNWANIVEVDYEKLNKGIGLATYLDQSVTKSARPGMIRVNLPDKDVKTIEPAFGKQYYYSTKGDDLHTKMETPLFDLTNATSAKFDFKSLYEIEAGYDFLEVHAVTEDGKQTLIERLGEKANSGNADSTNGKWIDKSYDLSQFKGKKVKLTFDYITDGGLALNGFALDNASLTVDGKVVFSDDAEGTPQLKLDGFVVSNGTEKKKHNYYVEWRNYAGADNALKFARGPVFNTGMVVWYADSAYTDNWVGVHPGHGFLGVVDSHPEAIVGTLNGKPTVKSSTRFQIADAAFSFDKTPAWKVVSPTRGTFTYDGLAGVPKFDDSKTYINQQIPDAGRILPKLGLKFEVVGQADDNSAGAVRLYR

SEQ ID NO: 78 - protein, 430 aa, Bacillus cereus
MKHNDCFDHNNCNPIVFSADCCKNPQSVPITREQLSQLITLLNSLVSAISAFFANPSNANRLVLLDLFNQFLIFLNSLLPSPEVNFLKQLTQSIIVLLQSPAPNLGQLSTLLQQFYSALAQFFFALDLIPISCNSNVDSATLQLLFNLLIQLINATPGATGPTGPTGPTGPTGPAGTGAGPTGATGATGATGPTGATGPAGTGGATGATGATGVTGATGATGATGPTGPTGATGPTGATGATGATGPTGATGPTGATGLTGATGAAGGGAIIPFASGTTPSALVNALVANTGTLLGFGFSQPGVALTGGTSITLALGVGDYAFVAPRAGTITSLAGFFSATAALAPISPVQVQIQILTAPAASNTFTVQGAPLLLTPAFAAIAIGSTASGIIAEAIPVAAGDKILLYVSLTAASPIAAVAGFVSAGINIV

SEQ ID NO: 79 - protein, 437 aa, Bacillus cereus
MKHNDCFGHNNCNNPIVFTPDCCNNPQTVPITSEQLGRLITLLNSLIAAIAAFFANPSDANRLALLNLFTQLLNLLNELAPSPEGNFLKQLIQSIINLLQSPNPNLGQLLSLLQQFYSALAPFFFSLILDPASLQLLLNLLAQLIGVTPGGGATGPTGPTGPGGGATGPTGPTGPGGGATGPTGATGPTGDTGLAGATGATGPTGDTGVAGPAGPTGPTGDTGLAGATGPTGPTGDTGLAGATGPTGATGLAGATGPTGATGLTGATGATGAAGGGAIIPFASGTTPAALVNALIANTGTLLGFGFSQPGIGLAGGTSITLALGVGDYAFVAPRDGVITSLAGFFSATAALSPLSPVQVQIQILTAPAASNTFTVQGAPLLLTPAFAAIAIGSTASGIIPEAIPVVAGDKILLYVSLTAASPIAAVAGFVSAGINIV

SEQ ID NO: 80 - protein, 119 aa, Bacillus anthracis
MLFTSWLLFFIFALAAFRLTRLIVYDKITGFLRRPFIDELEITEPDGSVSTFTKVKGKGLRKWIGELLSCYWCTGVWVSAFLLVLYNWIPIVAEPLLALLAIAGAAAIIETITGYFMGE

SEQ ID NO: 81 - protein, 61 aa, Bacillus anthracis
MFAVSNNPRQNSYDLQQWYHMQQQHQAQQQAYQEQLQQQGFVKKKGCNCGKKKSTIKHYEE

SEQ ID NO: 82 - protein, 481 aa, Bacillus anthracis
MSRYDDSQNKFSKPCFPSSAGRIPNTPSIPVTKAQLRTFRAIIIDLTKIIPKLFANPSPQNIEDLIDTLNLLSKFICSLDAASSLKAQGLAIIKNLITILKNPTFVASAVFIELQNLINYLLSITKLFRIDPCTLQELLKLIAALQTALVNSASFIQGPTGPTGPTGPTGPAGATGATGPQGVQGPAGATGATGPQGVQGPAGATGATGPQGAQGPAGATGATGPQGAQGPAGATGATGPQGIQGPAGATGATGPQGVQGPTGATGIGVTGPTGPSGGPAGATGPQGPQGNTGATGPQGIQGPAGATGATGPQGAQGPAGATGATGPQGVQGPTGATGIGVTGPTGPSGPSFPVATIVVTNNIQQTVLQFNNFIFNTAINVNNIIFNGTDTVTVINAGIYVISVSISTTAPGCAPLGVGISINGAVATDNFSSNLIGDSLSFTTIETLTAGANISVQSTLNEITIPATGNTNIRLTVFRIA

SEQ ID NO: 83 - protein, 275 aa, Bacillus thuringiensis
MKMKRGITTLLSVAVLSTSLVACSGITEKTVAKEEKVKLTDQQLMADLWYQTAGEMKALYYQGYNIGQLKLDAVLAKGTEKKPAIVLDLDETVLDNSPHQAMSVKTGKGYPYKWDDWINKAEAEALPGAIDFLKYTESKGVDIYYISNRKTNQLDATIKNLERVGAPQATKEHILLQDPKEKGKEKRRELVSQTHDIVLFFGDNLSDFTGFDGKSVKDRNQAVADSKAQFGEKFIIFPNPMYGDWEGALYDYDFKKSDAEKDKIRRDNLKSFDTK

SEQ ID NO: 84 - protein, 795 aa, Bacillus thuringiensis
MKKKKKLKPLAVLTTAAVLSSTFAFGGHAAYAETPTSSLPIDEHLIPEERLAEALKQRGVIDQSASQAETSKAVEKYVEKKKGENPGKEILTGDSLTQEASDFMKKVKDAKMRENEQAQQPEVGPVAGQGAALNPGKLNGKVPTTSAKQEEYNGAVRKDKVLVLLVEFSDFKHNNIDQEPGYMYSKDFNREHYQKMLFGDEPFTLFDGSKINTFKQYYEEQSGGSYTVDGTVTEWLTVPGKASDYGADAGTGHDNKGPLGPKDLVKEALKAAVAKGINLADFDQYDQYDQNGNGNKNEPDGIIDHLMVVHAGVGQEAGGGKLKDDAIWSHRSKLGSKPYAIDGTKSSVSNWGGKMAAYDYTIEPEDGAVGVFAHEYGHDLGLPDEYDTKYSGQGEPVESWSIMSGGSWAGKIAGTEPTSFSPQNKEFFQKNMKGNWANILEVDYDKLSKGIGVATYVDQSTTKSKRPGIVRVNLPDKDIKNIESAFGKKFYYSTKGNDIHTTLETPVFDLTNAKDAKFDYKAFYELEAKYDFLDVYAIAEDGTKTRIDRMGEKDIKGGADTTDGKWVDKSYDLSQFKGKKVKLQFEYLTDIAVAYKGFALDNAALTVDGKVVFSDDAEGQPAMTLKGFTVSNGFEQKKHNYYVEWRNYAGSDTALQYARGPVFNTGMVVWYADQSFTDNWVGVHPGEGFLGVVDSHPEAIVGTLNGQPTVKSSTRYQIADAAFSFDQTPAWKVNSPTRGIFDYKGLPGVAKFDDSKQYINSVIPDAGRKLPKLGLKFEVVGQAEDKSAGAVWLHR

SEQ ID NO: 85 - DNA, 169 bp, Bacillus anthracis
  source 1..169: mol_type = unassigned DNA
taatcaccctcttccaaatcaatcatatgttatacatatactaaactttccatttttttaaattgttcaagtagtttaagatttcttttcaataattcaaatgtccgtgtcattttctttcggttttgcatctactatataatgaacgctttatggaggtgaatttatg

SEQ ID NO: 86 - DNA, 303 bp, Bacillus anthracis
  source 1..303: mol_type = unassigned DNA
atttatttcattcaatttttcctatttagtacctaccgcactcacaaaaagcacctctcattaatttatattatagtcattgaaatctaatttaatgaaatcatcatactatatgttttataagaagtaaaggtaccatacttaattaatacatatctatacacttcaatatcacagcatgcagttgaattatatccaactttcatttcaaattaaataagtgcctccgctattgtgaatgtcatttactctccctactacatttaataattatgacaagcaatcataggaggttactacatg

SEQ ID NO: 87 - DNA, 173 bp, Bacillus anthracis
  source 1..173: mol_type = unassigned DNA
aattacataacaagaactacattagggagcaagcagtctagcgaaagctaactgcttttttattaaataactattttattaaatttcatatatacaatcgcttgtccatttcatttggctctacccacgcatttactattagtaatatgaatttttcagaggtggattttatt

SEQ ID NO: 88 - DNA, 124 bp, Bacillus weihenstephanensis
  source 1..124: mol_type = unassigned DNA; note = Bacillus weihenstephensis
ctatgatttaagatacacaatagcaaaagagaaacatattatataacgataaatgaaacttatgtatatgtatggtaactgtatatattactacaatacagtatactcataggaggtaggtatg

SEQ ID NO: 89 - DNA, 376 bp, Bacillus weihenstephanensis
  source 1..376: mol_type = unassigned DNA; note = Bacillus weihenstephensis
ggtaggtagatttgaaatatgatgaagaaaaggaataactaaaaggagtcgatatccgactccttttagttataaataatgtggaattagagtataattttatataggtatattgtattagatgaacgctttatcctttaattgtgattaatgatggattgtaagagaaggggcttacagtcctttttttatggtgttctataagcctttttaaaaggggtaccaccccacacccaaaaacagggggggttataactacatattggatgttttgtaacgtacaagaatcggtattaattaccctgtaaataagttatgtgtatataaggtaactttatatattctcctacaataaaataaaggaggtaataaagtg

SEQ ID NO: 90 - DNA, 225 bp, Bacillus thuringiensis
  source 1..225: mol_type = unassigned DNA
aacccttaatgcattggttaaacattgtaaagtctaaagcatggataatgggcgagaagtaagtagattgttaacaccctgggtcaaaaattgatatttagtaaaattagttgcactttgtgcattttttcataagatgagtcatatgttttaaattgtagtaatgaaaaacagtattatatcataatgaattggtatcttaataaaagagatggaggtaactta

SEQ ID NO: 91 - DNA, 125 bp, Bacillus thuringiensis
  source 1..125: mol_type = unassigned DNA
taattccaccttcccttatcctctttcgcctatttaaaaaaaggtcttgagattgtgaccaaatctcctcaactccaatatcttattaatgtaaatacaaacaagaagataaggagtgacattaa

SEQ ID NO: 92 - DNA, 144 bp, Bacillus thuringiensis
  source 1..144: mol_type = unassigned DNA
aggatgtctttttttatattgtattatgtacatccctactatataaattccctgcttttatcgtaagaattaacgtaatatcaaccatatcccgttcatattgtagtagtgtatgtcagaactcacgagaaggagtgaacataa

SEQ ID NO: 93 - DNA, 126 bp, Bacillus thuringiensis
  source 1..126: mol_type = unassigned DNA
ttaatgtcactccttatcttcttgtttgtatttacattaataagatattggagttgaggagatttggtcacaatctcaagaccttttttttaaataggcgaaagaggataagggaaggtggaatta

SEQ ID NO: 94 - DNA, 103 bp, Bacillus thuringiensis
  source 1..103: mol_type = unassigned DNA
atatattttcataatacgagaaaaagcggagtttaaaagaatgagggaacggaaataaagagttgttcatatagtaaatagacagaattgacagtagaggaga

SEQ ID NO: 95 - DNA, 169 bp, Bacillus thuringiensis
  source 1..169: mol_type = unassigned DNA
aaactaaataatgagctaagcatggattgggtggcagaattatctgccacccaatccatgcttaacgagtattattatgtaaatttcttaaaattgggaacttgtctagaacatagaacctgtccttttcattaactgaaagtagaaacagataaaggagtgaaaaaca

SEQ ID NO: 96 - DNA, 111 bp, Bacillus thuringiensis
  source 1..111: mol_type = unassigned DNA
attcactacaacggggatgagtttgatgcggatacatatgagaagtaccggaaagtgtttgtagaacattacaaagatatattatctccatcataaaggagagatgcaaag

SEQ ID NO: 97 - DNA, 273 bp, Bacillus anthracis
  source 1..273: mol_type = unassigned DNA
cgcgcaccacttcgtcgtacaacaacgcaagaagaagttggggatacagcagtattcttattcagtgatttagcacgcggcgtaacaggagaaaacattcacgttgattcagggtatcatatcttaggataaatataatattaattttaaaggacaatctctacatgttgagattgtcctttttatttgttcttagaaagaacgatttttaacgaaagttcttaccacgttatgaatataagtataatagtacacgatttattcagctacgta

SEQ ID NO: 98 - DNA, 303 bp, Bacillus anthracis
  source 1..303: mol_type = unassigned DNA
tgaagtatctagagctaatttacgcaaaggaatctcaggacaacactttcgcaacacctatattttaaatttaataaaaaaagagactccggagtcagaaattataaagctagctgggttcaaatcaaaaatttcactaaaacgatattatcaatacgcagaaaatggaaaaaacgccttatcataaggcgttttttccattttttcttcaaacaaacgattttactatgaccatttaactaatttttgcatctactatgatgagtttcattcacattctcattagaaaggagagatttaatg

SEQ ID NO: 99 - DNA, 240 bp, Bacillus anthracis
  source 1..240: mol_type = unassigned DNA
tatatcatatgtaaaattagttcttattcccacatatcatatagaatcgccatattatacatgcagaaaactaagtatggtattattcttaaattgtttagcaccttctaatattacagatagaatccgtcattttcaacagtgaacatggatttcttctgaacacaactctttttctttccttatttccaaaaagaaaagcagcccattttaaaatacggctgcttgtaatgtacatta

SEQ ID NO: 100 - DNA, 267 bp, Bacillus thuringiensis
  source 1..267: mol_type = unassigned DNA
tatcacataactctttatttttaatatttcgacataaagtgaaactttaatcagtgggggctttgttcatccccccactgattattaattgaaccaagggataaaaagatagagggtctgaccagaaaactggagggcatgattctataacaaaaagcttaatgtttatagaattatgtctttttatatagggagggtagtaaacagagatttggacaaaaatgcaccgatttatctgaattttaagttttataaaggggagaaatg

SEQ ID NO: 101 - DNA, 124 bp, Bacillus thuringiensis
  source 1..124: mol_type = unassigned DNA
attttttacttagcagtaaaactgatatcagttttactgctttttcatttttaaattcaatcattaaatcttccttttctacatagtcataatgttgtatgacattccgtaggaggcacttata

SEQ ID NO: 102 - DNA, 170 bp, Bacillus thuringiensis
  source 1..170: mol_type = unassigned DNA
acataaattcacctccataaagcgttcattatatagtagatgcaaaaccgaaagaaaatgacacggacatttgaattattgaaaagaaatcttaaactacttgaacaatttaaaaaaatggaaagtttagtatatgtataacatatgattgatttggaagagggtgatta

SEQ ID NO: 103 - DNA, 212 bp, Bacillus thuringiensis
  source 1..212: mol_type = unassigned DNA
ttctattttccaacataacatgctacgattaaatggttttttgcaaatgccttcttgggaagaaggattagagcgtttttttatagaaaccaaaagtcattaacaattttaagttaatgacttttttgtttgcctttaagaggttttatgttactataattatagtatcaggtactaataacaagtataagtatttctgggaggatatatca

SEQ ID NO: 104 - DNA, 1500 bp, Bacillus subtilis
  source 1..1500: mol_type = unassigned DNA
atgaaacggtcaatctcgatttttattacgtgtttattgattacgttattgacaatgggcggcatgatagcttcgccggcatcagcagcagggacaaaaacgccagtagccaagaatggccagcttagcataaaaggtacacagctcgttaaccgagacggtaaagcggtacagctgaaggggatcagttcacacggattgcaatggtatggagaatatgtcaataaagacagcttaaaatggctgagagatgattggggtatcaccgttttccgtgcagcgatgtatacggcagatggcggttatattgacaacccgtccgtgaaaaataaagtaaaagaagcggttgaagcggcaaaagagcttgggatatatgtcatcattgactggcatatcttaaatgacggtaatccaaaccaaaataaagagaaggcaaaagaattcttcaaggaaatgtcaagcctttacggaaacacgccaaacgtcatttatgaaattgcaaacgaaccaaacggtgatgtgaactggaagcgtgatattaaaccatatgcggaagaagtgatttcagttatccgcaaaaatgatccagacaacatcatcattgtcggaaccggtacatggagccaggatgtgaatgatgctgccgatgaccagctaaaagatgcaaacgttatgtacgcacttcatttttatgccggcacacacggccaatttttacgggataaagcaaactatgcactcagcaaaggagcacctatttttgtgacagagtggggaacaagcgacgcgtctggcaatggcggtgtattccttgatcaatcgagggaatggctgaaatatctcgacagcaagaccattagctgggtgaactggaatctttctgataagcaggaatcatcctcagctttaaagccgggggcatctaaaacaggcggctggcggttgtcagatttatctgcttcaggaacattcgttagagaaaacattctcggcaccaaagattcgacgaaggacattcctgaaacgccatcaaaagataaacccacacaggaaaatggtatttctgtacagtacagagcaggggatgggagtatgaacagcaaccaaatccgtccgcagcttcaaataaaaaataacggcaataccacggttgatttaaaagatgtcactgcccgttactggtataaagcgaaaaacaaaggccaaaactttgactgtgactacgcgcagattggatgcggcaatgtgacacacaagtttgtgacgttgcataaaccaaagcaaggtgcagatacctatctggaacttggatttaaaaacggaacgttggcaccgggagcaagcacagggaatattcagctccgtcttcacaatgatgactggagcaattatgcacaaagcggcgattattcctttttcaaatcaaatacgtttaaaacaacgaaaaaaatcacattatatgatcaaggaaaactgatttggggaacagaaccaaattag

SEQ ID NO: 105 - DNA, 852 bp, Bacillus thuringiensis
  source 1..852: mol_type = unassigned DNA
atgaaaaagaaagtacttgctttagcggcagctattacattggttgctccattacaaagtgttgcatttgctcatgaaaatgatgggggacagagatttggagttattccgcgctggtctgctgaagataaacataaagaaggcgtgaattctcatttatggattgtaaatcgtgcaattgatattatgtctcgtaatacaacacttgtaaaacaagatcgagttgcactattaaatgaatggcgtactgagttagagaacggtatttatgctgctgactatgaaaatccttattatgataatagcacatttgcttcacatttctatgaccctgacaatgggaaaacttatattccgtatgcaaagcaggcaaaggaaactggagctaaatattttaaattagctggtgagtcttacaaaaataaagatatgcaacaagcattcttctatttaggattatctcttcattatctaggggatgtaaaccaaccgatgcatgcagcaaactttacaaacctttcgtatccacaagggttccattctaaatatgaaaactttgtagatacgataaaagataactataaagtaacggatggaaatggatattggaactggaaaggtacgaatccagaagattggattcatggagcggcagtagttgcgaaacaagattacgctggcattgtaaatgataatacgaaagattggttcgtgagagctgctgtatcacaagaatatgcagataaatggcgcgctgaagttacaccaatgacaggtaagcgtttaatggatgcacaacgtgttactgctggatatattcagctttggtttgatacgtacggagatcgttaa

SEQ ID NO: 106 - DNA, 729 bp, Bacillus subtilis
  source 1..729: mol_type = unassigned DNA
gcgggactgaataaagatcaaaagcgccgggcggaacagctgacaagtatctttgaaaacggcacaacggagatccaatatggatatgtagagcgattggatgacgggcgaggctatacatgcggacgggcaggctttacaacggctaccggggatgcattggaagtagtggaagtatacacaaaggcagttccgaataacaaactgaaaaagtatctgcctgaattgcgccgtctggccaaggaagaaagcgatgatacaagcaatctcaagggattcgcttctgcctggaagtcgcttgcaaatgataaggaatttcgcgccgctcaagacaaagtaaatgaccatttgtattatcagcctgccatgaaacgatcggataatgccggactaaaaacagcattggcaagagctgtgatgtacgatacggttattcagcatggcgatggtgatgaccctgactctttttatgccttgattaaacgtacgaacaaaaaagcgggcggatcacctaaagacggaatagacgagaagaagtggttgaataaattcttggacgtacgctatgacgatctgatgaatccggccaatcatgacacccgtgacgaatggagagaatcagttgcccgtgtggacgtgcttcgctctatcgccaaggagaacaactataatctaaacggaccgattcatgttcgttcaaacgagtacggtaattttgtaatcaaataa

SEQ ID NO: 107 - protein, 499 aa, Bacillus subtilis
MKRSISIFITCLLITLLTMGGMIASPASAAGTKTPVAKNGQLSIKGTQLVNRDGKAVQLKGISSHGLQWYGEYVNKDSLKWLRDDWGITVFRAAMYTADGGYIDNPSVKNKVKEAVEAAKELGIYVIIDWHILNDGNPNQNKEKAKEFFKEMSSLYGNTPNVIYEIANEPNGDVNWKRDIKPYAEEVISVIRKNDPDNIIIVGTGTWSQDVNDAADDQLKDANVMYALHFYAGTHGQFLRDKANYALSKGAPIFVTEWGTSDASGNGGVFLDQSREWLKYLDSKTISWVNWNLSDKQESSSALKPGASKTGGWRLSDLSASGTFVRENILGTKDSTKDIPETPSKDKPTQENGISVQYRAGDGSMNSNQIRPQLQIKNNGNTTVDLKDVTARYWYKAKNKGQNFDCDYAQIGCGNVTHKFVTLHKPKQGADTYLELGFKNGTLAPGASTGNIQLRLHNDDWSNYAQSGDYSFFKSNTFKTTKKITLYDQGKLIWGTEPN

SEQ ID NO: 108 - protein, 283 aa, Bacillus thuringiensis
MKKKVLALAAAITLVAPLQSVAFAHENDGGQRFGVIPRWSAEDKHKEGVNSHLWIVNRAIDIMSRNTTLVKQDRVALLNEWRTELENGIYAADYENPYYDNSTFASHFYDPDNGKTYIPYAKQAKETGAKYFKLAGESYKNKDMQQAFFYLGLSLHYLGDVNQPMHAANFTNLSYPQGFHSKYENFVDTIKDNYKVTDGNGYWNWKGTNPEDWIHGAAVVAKQDYAGIVNDNTKDWFVRAAVSQEYADKWRAEVTPMTGKRLMDAQRVTAGYIQLWFDTYGDR

SEQ ID NO: 109 - protein, 244 aa, Bacillus subtilis
LEAGLNKDQKRRAEQLTSIFENGTTEIQYGYVERLDDGRGYTCGRAGFTTATGDALEVVEVYTKAVPNNKLKKYLPELRRLAKEESDDTSNLKGFASAWKSLANDKEFRAAQDKVNDHLYYQPAMKRSDNAGLKTALARAVMYDTVIQHGDGDDPDSFYALIKRTNKKAGGSPKDGIDEKKWLNKFLDVRYDDLMNPANHDTRDEWRESVARVDVLRSIAKENNYNLNGPIHVRSNEYGNFVIK

(19) United States
(12) Patent Application Publication
(10) Pub. No.: US 2024/0285744 A1
(43) Pub. Date: Aug. 29, 2024

(54) COMPOSITIONS COMPRISING RECOMBINANT BACILLUS CELLS AND AN INSECTICIDE

(71) Applicants: Spogen Biotech Inc., St. Louis, MO (US); Bayer CropScience LP, St. Louis, MO (US)

(72) Inventors: Damian Curtis, Davis, CA (US); Brian Thompson, Creve Coeur, MO (US)

(21) Appl. No.: 18/644,678

(22) Filed: Apr. 24, 2024

Related U.S. Application Data
(62) Division of application No. 17/356,958, filed on Jun. 24, 2021, now Pat. No. 12,016,912.
Application No. 17/356,958 is a continuation of application No. 15/511,864, filed on Mar. 16, 2017, now Pat. No. 11,044,916.
(60) Provisional application No. 62/051,935, filed on Sep. 17, 2014.

(51) Int. Cl. A61K 39/07; A61K 35/00; A61K 35/742

(52) U.S. Cl. CPC A61K 39/07; A61K 35/742; C12Y 301/01; C12Y 302/01004; C12Y 302/01132; A61K 2035/115; Y02A 50/30

(57) ABSTRACT

The present invention relates to a composition comprising a) recombinant exosporium-producing Bacillus cells that express a fusion protein comprising: (i) at least one plant growth stimulating protein or peptide and (ii) a targeting sequence that localizes the fusion protein to the exosporium of the Bacillus cells; and b) at least one particular insecticide disclosed herein in a synergistically effective amount. Furthermore, the present invention relates to the use of this composition as well as a method for enhancing plant growth, promoting plant health, and/or reducing overall damage of plants and plant parts.

### CROSS REFERENCE TO RELATED APPLICATIONS

This application is a divisional of U.S. patent application Ser. No. 17/356,958, filed Jun. 24, 2021, which is a continuation of U.S. patent application Ser. No. 15/511,864, filed Mar. 16, 2017, now U.S. Pat. No. 11,044,916, which is a 371 National Stage Entry of PCT/US2015/050658, filed on Sep. 17, 2015, which claims priority to U.S. Provisional Patent Application No. 62/051,935, filed Sep. 17, 2014, each of which is incorporated herein by reference in its entirety.

### REFERENCE TO SEQUENCE LISTING SUBMITTED ELECTRONICALLY

The official copy of the sequence listing is submitted electronically via Patent Center with a file named “LMNE111USC1D1_ST26.xml” created on Apr. 19, 2024, and having a size of 119 kilobytes (as measured in MS-Windows®), is filed concurrently with the specification, and is herein incorporated by reference in its entirety.

### BACKGROUND

### Field of the Invention

The present invention relates to a composition comprising (i) recombinant exosporium-producing Bacillus cells that express a fusion protein comprising: (x) at least one plant growth stimulating protein or peptide; and (y) a targeting sequence that localizes the fusion protein to the exosporium of the Bacillus cells; and (ii) at least one insecticide selected from the particular insecticides disclosed herein that exhibits the ability to improve plant growth and/or health and/or activity against insects, mites, nematodes and/or phytopathogens in synergistically effective amounts. Furthermore, the present invention relates to the use of this composition as well as a method for enhancing plant growth, promoting plant health, and/or reducing overall damage of plants and plant parts.

### Background of the Invention

In crop protection, there is a continuous need for applications that improve the health and/or the growth of plants. Healthier plants generally result in higher yields and/or better quality of a plant or its products.

In order to promote plant health, fertilizers are employed worldwide, based on both inorganic and organic substances. A fertilizer may be a single substance or a composition, and is used to provide nutrients to plants. A major breakthrough in the application of fertilizers was the development of nitrogen-based fertilizer by Justus von Liebig around 1840. Fertilizers, however, can lead to soil acidification and destabilization of nutrient balance in soil, including depletion of minerals and enrichment of salt and heavy metals. In addition, excessive fertilizer use can lead to alteration of soil fauna as well as contaminate surface water and ground water. Further, unhealthful substances such as nitrate may become enriched in plants and fruits.

In addition, insecticides and fungicide are employed worldwide to control pests. Synthetic insecticides or fungicides often are non-specific and therefore can act on organisms other than the target organisms, including other naturally occurring beneficial organisms. Because of their chemical nature, they may also be toxic and non-biodegradable. Consumers worldwide are increasingly conscious of the potential environmental and health problems associated with the residuals of chemicals, particularly in food products. This has resulted in growing consumer pressure to reduce the use or at least the quantity of chemical (i.e., synthetic) pesticides. Thus, there is a need to manage food chain requirements while still allowing effective pest control.

A further problem arising with the use of synthetic insecticides or fungicides is that the repeated and exclusive application of an insecticide or fungicides often leads to selection of resistant animal pests or microorganisms. Normally, such strains are also cross-resistant against other active ingredients having the same mode of action. An effective control of the pathogens with said active compounds is then not possible any longer. However, active ingredients having new mechanisms of action are difficult and expensive to develop.

The use of biological control agents (BCAs), which act as plant health-enhancing and/or plant protection agents, is an alternative to fertilizers and synthetic pesticides. In some cases, the effectiveness of BCAs is not at the same level as for conventional insecticides and fungicides, especially in case of severe infection pressure. Consequently, in some circumstances, biological control agents, their mutants and metabolites produced by them are, in particular in low application rates, not entirely satisfactory. Thus, there is a constant need for developing new plant health-enhancing and/or plant protection compositions, including biological control agents used in conjunction with synthetic fungicides and insecticides, to strive to fulfill the above-mentioned requirements.

### SUMMARY

In view of this, it was in particular an object of the present invention to provide compositions which have an enhanced ability to improve plant growth and/or to enhance plant health or which exhibit enhanced activity against insects, mites, nematodes and/or phytopathogens.

Accordingly, it was found that these objectives are achieved with the compositions according to the invention as defined in the following. By applying a) recombinant exosporium-producing Bacillus cells that express a fusion protein comprising: (i) at least one plant growth stimulating protein or peptide selected from the group consisting of an enzyme involved in the production or activation of a plant growth stimulating compound; an enzyme that degrades or modifies a bacterial, fungal, or plant nutrient source; and a protein or peptide that protects a plant from a pathogen or a pest; and (ii) a targeting sequence that localizes the fusion protein to the exosporium of the Bacillus cells; and b) at least one particular insecticide disclosed herein, one is able to enhance preferably in a superadditive manner (i) plant growth, plant yield and/or plant health and/or (ii) the activity against insects, mites, nematodes and/or phytopathogens.

References herein to targeting sequences, exosporium proteins, exosporium protein fragments, fusion proteins, and recombinant exosporium producing Bacillus cells that express such fusion proteins should not be considered to be stand-alone embodiments. Instead, throughout the present application, references to the targeting sequences, exosporium proteins, exosporium protein fragments, fusion proteins, and recombinant exosporium producing Bacillus cells that express such fusion proteins should be considered to be disclosed and claimed only in combination (and preferably in a synergistic combination) with one or more of the particular insecticides described herein. Furthermore, references to the “particular insecticide disclosed herein” are intended to encompass insecticides described below in paragraphs [000185]-[000212].

The present invention is directed to a composition comprising in a synergistically effective amount: a) recombinant exosporium-producing Bacillus cells that express a fusion protein comprising: (i) at least one plant growth stimulating protein or peptide selected from the group consisting of an enzyme involved in the production or activation of a plant growth stimulating compound; an enzyme that degrades or modifies a bacterial, fungal, or plant nutrient source; and a protein or peptide that protects a plant from a pathogen; and (ii) a targeting sequence that localizes the fusion protein to the exosporium of the Bacillus cells; and b) at least one insecticide selected from the group consisting of abamectin, carbofuran, cyazypyr, cycloaprid, chlorantraniliprole, spinosad, sulfoxaflor, tefluthrin, and thiamethoxam.

In some embodiments, the targeting sequence comprises: an amino acid sequence having at least about 43% identity with amino acids 20-35 of SEQ ID NO: 1, wherein the identity with amino acids 25-35 is at least about 54%; a targeting sequence comprising amino acids 1-35 of SEQ ID NO: 1; a targeting sequence comprising amino acids 20-35 of SEQ ID NO: 1; a targeting sequence comprising amino acids 22-31 of SEQ ID NO: 1; a targeting sequence comprising amino acids 22-33 of SEQ ID NO: 1; a targeting sequence comprising amino acids 20-31 of SEQ ID NO: 1; a targeting sequence comprising SEQ ID NO: 1; or an exosporium protein comprising an amino acid sequence having at least 85% identity with SEQ ID NO: 2.

In some embodiments, the exosporium-producing Bacillus cells are cells of a Bacillus cereus family member. The recombinant exosporium-producing Bacillus cells may be any one of Bacillus anthracis, Bacillus cereus, Bacillus thuringiensis, Bacillus mycoides, Bacillus pseudomycoides, Bacillus samanii, Bacillus gaemokensis, Bacillus weihenstephensis, Bacillus toyoiensis and combinations thereof. In a further embodiment, the recombinant Bacillus cells are cells of Bacillus thuringiensis BT013A.

In certain aspects, the fusion protein comprises an enzyme involved in the production or activation of a plant growth stimulating compound selected from the group consisting of an acetoin reductase, an indole-3-acetamide hydrolase, a tryptophan monooxygenase, an acetolactate synthetase, an α-acetolactate decarboxylase, a pyruvate decarboxylase, a diacetyl reductase, a butanediol dehydrogenase, an aminotransferase, a tryptophan decarboxylase, an amine oxidase, an indole-3-pyruvate decarboxylase, an indole-3-acetaldehyde dehydrogenase, a tryptophan side chain oxidase, a nitrile hydrolase, a nitrilase, a peptidase, a protease, an adenosine phosphate isopentenyltransferase, a phosphatase, an adenosine kinase, an adenine phosphoribosyltransferase, CYP735A, a 5′ribonucleotide phosphohydrolase, an adenosine nucleosidase, a zeatin cis-trans isomerase, a zeatin O-glucosyltransferase, a β-glucosidase, a cis-hydroxylase, a CK cis-hydroxylase, a CK N-glucosyltransferase, a 2,5-ribonucleotide phosphohydrolase, an adenosine nucleosidase, a purine nucleoside phosphorylase, a zeatin reductase, a hydroxylamine reductase, a 2-oxoglutarate dioxygenase, a gibberellic 2B/3B hydrolase, a gibberellin 3-oxidase, a gibberellin 20-oxidase, a chitosanase, a chitinase, a β-1,3-glucanase, a β-1,4-glucanase, a β-1,6-glucanase, an aminocyclopropane-1-carboxylic acid deaminase, and an enzyme involved in producing a nod factor.

In other aspects, the fusion protein comprises an enzyme that degrades or modifies a bacterial, fungal, or plant nutrient source selected from the group consisting of a cellulase, a lipase, a lignin oxidase, a protease, a glycoside hydrolase, a phosphatase, a nitrogenase, a nuclease, an amidase, a nitrate reductase, a nitrite reductase, an amylase, an ammonia oxidase, a ligninase, a glucosidase, a phospholipase, a phytase, a pectinase, a glucanase, a sulfatase, a urease, a xylanase, and a siderophore.

In still other aspects, the fusion protein comprises a protein or peptide that protects a plant from a pathogen and the protein or peptide has insecticidal activity, helminthicidal activity, suppresses insect or worm predation, or a combination thereof. Such a protein may comprise an insecticidal bacterial toxin, an endotoxin, a Cry toxin, a protease inhibitor protein or peptide, a cysteine protease, or a chitinase. The protein or peptide may comprise a VIP insecticidal toxin, a trypsin inhibitor, an arrowhead protease inhibitor, a Cry toxin (e.g., a Cry toxin from Bacillus thuringiensis).

In some embodiments, the at least one insecticide is selected from the group consisting of abamectin, carbofuran, cyazypyr, cycloaprid, chlorantraniliprole, spinosad, sulfoxaflor, tefluthrin, and thiamethoxam.

In some embodiments, the composition of the present invention comprises a) recombinant exosporium-producing Bacillus cells that express a fusion protein comprising: (i) at least one plant growth stimulating protein or peptide selected from the group consisting of an enzyme involved in the production or activation of a plant growth stimulating compound and an enzyme that degrades or modifies a bacterial, fungal, or plant nutrient source or at least one protein or peptide that protects a plant from a pathogen; and (ii) a targeting sequence that localizes the fusion protein to the exosporium of the Bacillus cells; and b) at least one insecticide selected from the group consisting of abamectin, carbofuran, cyazypyr, cycloaprid, chlorantraniliprole, spinosad, sulfoxaflor, tefluthrin, and thiamethoxam in a synergistically effective amount.

In a particular aspect of the above embodiments (i) the at least one insecticide is abamectin; (ii) the targeting sequence comprises an amino acid sequence having at least about 43% identity with amino acids 20-35 of SEQ ID NO: 1, wherein the identity with amino acids 25-35 is at least about 54%; (iii) the plant growth stimulating protein or peptide comprises endoglucanase, phospholipase or chitosinase, preferably with at least 95% sequence identity to SEQ ID NO: 107, 108 and 109, respectively; and (iv) the recombinant Bacillus cereus family member cells comprise cells of Bacillus thuringiensis or Bacillus mycoides. In yet another particular embodiment, the recombinant Bacillus cereus family member cells are cells of Bacillus thuringiensis BT013A.

In a particular aspect of the above embodiments (i) the at least one insecticide is carbofuran; (ii) the targeting sequence comprises an amino acid sequence having at least about 43% identity with amino acids 20-35 of SEQ ID NO: 1, wherein the identity with amino acids 25-35 is at least about 54%; (iii) the plant growth stimulating protein or peptide comprises endoglucanase, phospholipase or chitosinase, preferably with at least 95% sequence identity to SEQ ID NO: 107, 108 and 109, respectively; and (iv) the recombinant Bacillus cereus family member cells comprise cells of Bacillus thuringiensis or Bacillus mycoides. In yet another particular embodiment, the recombinant Bacillus cereus family member cells are cells of Bacillus thuringiensis BT013A.

In a particular aspect of the above embodiments (i) the at least one insecticide is cyazypyr; (ii) the targeting sequence comprises an amino acid sequence having at least about 43% identity with amino acids 20-35 of SEQ ID NO: 1, wherein the identity with amino acids 25-35 is at least about 54%; (iii) the plant growth stimulating protein or peptide comprises endoglucanase, phospholipase or chitosinase, preferably with at least 95% sequence identity to SEQ ID NO: 107, 108 and 109, respectively; and (iv) the recombinant Bacillus cereus family member cells comprise cells of Bacillus thuringiensis or Bacillus mycoides. In yet another particular embodiment, the recombinant Bacillus cereus family member cells are cells of Bacillus thuringiensis BT013A.

In a particular aspect of the above embodiments (i) the at least one insecticide is cycloaprid; (ii) the targeting sequence comprises an amino acid sequence having at least about 43% identity with amino acids 20-35 of SEQ ID NO: 1, wherein the identity with amino acids 25-35 is at least about 54%; (iii) the plant growth stimulating protein or peptide comprises endoglucanase, phospholipase or chitosinase, preferably with at least 95% sequence identity to SEQ ID NO: 107, 108 and 109, respectively; and (iv) the recombinant Bacillus cereus family member cells comprise cells of Bacillus thuringiensis or Bacillus mycoides. In yet another particular embodiment, the recombinant Bacillus cereus family member cells are cells of Bacillus thuringiensis BT013A.

In a particular aspect of the above embodiments (i) the at least one insecticide is chlorantraniliprole; (ii) the targeting sequence comprises an amino acid sequence having at least about 43% identity with amino acids 20-35 of SEQ ID NO: 1, wherein the identity with amino acids 25-35 is at least about 54%; (iii) the plant growth stimulating protein or peptide comprises endoglucanase, phospholipase or chitosinase, preferably with at least 95% sequence identity to SEQ ID NO: 107, 108 and 109, respectively; and (iv) the recombinant Bacillus cereus family member cells comprise cells of Bacillus thuringiensis or Bacillus mycoides. In yet another particular embodiment, the recombinant Bacillus cereus family member cells are cells of Bacillus thuringiensis BT013A.

In a particular aspect of the above embodiments (i) the at least one insecticide is spinosad; (ii) the targeting sequence comprises an amino acid sequence having at least about 43% identity with amino acids 20-35 of SEQ ID NO: 1, wherein the identity with amino acids 25-35 is at least about 54%; (iii) the plant growth stimulating protein or peptide comprises endoglucanase, phospholipase or chitosinase, preferably with at least 95% sequence identity to SEQ ID NO: 107, 108 and 109, respectively; and (iv) the recombinant Bacillus cereus family member cells comprise cells of Bacillus thuringiensis or Bacillus mycoides. In yet another particular embodiment, the recombinant Bacillus cereus family member cells are cells of Bacillus thuringiensis BT013A.

In a particular aspect of the above embodiments (i) the at least one insecticide is sulfoxaflor; (ii) the targeting sequence comprises an amino acid sequence having at least about 43% identity with amino acids 20-35 of SEQ ID NO: 1, wherein the identity with amino acids 25-35 is at least about 54%; (iii) the plant growth stimulating protein or peptide comprises endoglucanase, phospholipase or chitosinase, preferably with at least 95% sequence identity to SEQ ID NO: 107, 108 and 109, respectively; and (iv) the recombinant Bacillus cereus family member cells comprise cells of Bacillus thuringiensis or Bacillus mycoides. In yet another particular embodiment, the recombinant Bacillus cereus family member cells are cells of Bacillus thuringiensis BT013A.

In a particular aspect of the above embodiments (i) the at least one insecticide is tefluthrin; (ii) the targeting sequence comprises an amino acid sequence having at least about 43% identity with amino acids 20-35 of SEQ ID NO: 1, wherein the identity with amino acids 25-35 is at least about 54%; (iii) the plant growth stimulating protein or peptide comprises endoglucanase, phospholipase or chitosinase, preferably with at least 95% sequence identity to SEQ ID NO: 107, 108 and 109, respectively; and (iv) the recombinant Bacillus cereus family member cells comprise cells of Bacillus thuringiensis or Bacillus mycoides. In yet another particular embodiment, the recombinant Bacillus cereus family member cells are cells of Bacillus thuringiensis BT013A.

In a particular aspect of the above embodiments (i) the at least one insecticide is thiamethoxam; (ii) the targeting sequence comprises an amino acid sequence having at least about 43% identity with amino acids 20-35 of SEQ ID NO: 1, wherein the identity with amino acids 25-35 is at least about 54%; (iii) the plant growth stimulating protein or peptide comprises endoglucanase, phospholipase or chitosinase, preferably with at least 95% sequence identity to SEQ ID NO: 107, 108 and 109, respectively; and (iv) the recombinant Bacillus cereus family member cells comprise cells of Bacillus thuringiensis or Bacillus mycoides. In yet another particular embodiment, the recombinant Bacillus cereus family member cells are cells of Bacillus thuringiensis BT013A.

In yet other embodiments, the composition further comprises at least one fungicide. The at least one fungicide may be synthetic.

In some aspects, the composition further comprises at least one auxiliary selected from the group consisting of extenders, solvents, spontaneity promoters, carriers, emulsifiers, dispersants, frost protectants, thickeners and adjuvants.

In other aspects, the invention is directed to a seed treated with any of the compositions disclosed herein.

Furthermore, the present invention relates to use of the disclosed compositions as an insecticide and/or fungicide. In certain aspects, the disclosed compositions are used for reducing overall damage of plants and plant parts as well as losses in harvested fruits or vegetables caused by insects, mites, nematodes and/or phytopathogens. In other aspects, the disclosed compositions are used for enhancing plant growth and/or promoting plant health.

Additionally, the present invention is directed to a method of treating a plant, a plant part, such as a seed, root, rhizome, corm, bulb, or tuber, and/or a locus on which or near which the plant or the plant parts grow, such as soil, to enhance plant growth and/or promote plant health comprising the step of simultaneously or sequentially applying to a plant, a plant part and/or a plant loci: a) recombinant exosporium-producing Bacillus cells that express a fusion protein comprising: (i) at least one plant growth stimulating protein or peptide selected from the group consisting of an enzyme involved in the production or activation of a plant growth stimulating compound; an enzyme that degrades or modifies a bacterial, fungal, or plant nutrient source; and a protein or peptide that protects a plant from a pathogen; and (ii) a targeting sequence that localizes the fusion protein to the exosporium of the Bacillus cells; and b) at least one insecticide selected from particular insecticide disclosed herein that exhibits activity against insects, mites, nematodes and/or phytopathogens in a synergistically effective amount.

In another embodiment, the present invention is a method for reducing overall damage of plants and plant parts as well as losses in harvested fruits or vegetables caused by insects, mites, nematodes and/or phytopathogens comprising the step of simultaneously or sequentially applying to a plant, a plant part, such as a seed, root, rhizome, corm, bulb, or tuber, and/or a locus on which or near which the plant or the plant parts grow, such as soil: a) recombinant exosporium-producing Bacillus cells that express a fusion protein comprising: (i) at least one plant growth stimulating protein or peptide selected from the group consisting of an enzyme involved in the production or activation of a plant growth stimulating compound; an enzyme that degrades or modifies a bacterial, fungal, or plant nutrient source; and a protein or peptide that protects a plant from a pathogen; and (ii) a targeting sequence that localizes the fusion protein to the exosporium of the Bacillus cells; and b) at least one insecticide selected from the particular insecticides disclosed herein that exhibits activity against insects, mites, nematodes and/or phytopathogens in a synergistically effective amount.

In the above paragraphs, the term “comprise” or any derivative thereof (e.g., comprising, comprises) may be replaced with “consist of” or the applicable corresponding derivative thereof.

### BRIEF DESCRIPTION OF THE DRAWINGS

FIG. 1 shows an alignment of the amino acid sequence of the amino-terminal portion of Bacillus anthracis Sterne strain BclA and with the corresponding region from various exosporium proteins from Bacillus cereus family members.

### DETAILED DESCRIPTION

In general “pesticidal” means the ability of a substance to increase mortality or inhibit the growth rate of plant pests. The term is used herein, to describe the property of a substance to exhibit activity against insects, mites, nematodes and/or phytopathogens. In the sense of the present invention the term “pests” include insects, mites, nematodes and/or phytopathogens.

NRRL is the abbreviation for the Agricultural Research Service Culture Collection, having the address National Center for Agricultural Utilization Research, Agricultural Research Service, U.S. Department of Agriculture, 1815 North University Street, Peoria, Illinois 61604, U.S.A.

ATCC is the abbreviation for the American Type Culture Collection, having the address ATCC Patent Depository, 10801 University Boulevard, Manassas, Virginia 10110, U.S.A.

All strains described herein and having an accession number in which the prefix is NRRL or ATCC have been deposited with the above-described respective depositary institution in accordance with the Budapest Treaty on the International Recognition of the Deposit of Microorganisms for the Purposes of Patent Procedure.

An “enzyme involved in the production or activation of a plant growth stimulating compound” includes any enzyme that catalyzes any step in a biological synthesis pathway for a compound that stimulates plant growth or alters plant structure, or any enzyme that catalyzes the conversion of an inactive or less active derivative of a compound that stimulates plant growth or alters plant structure to an active or more active form of the compound. Such compounds include, for example, but are not limited to, small molecule plant hormones such as auxins and cytokinins, bioactive peptides, and small plant growth stimulating molecules synthesized by bacteria or fungi in the rhizosphere (e.g., 2,3-butanediol).

A “plant immune system enhancer protein or peptide” as used herein includes any protein or peptide that has a beneficial effect on the immune system of a plant.

The term “plant growth stimulating protein or peptide” as used herein includes any protein or peptide that increases plant growth in a plant exposed to the protein or peptide.

The terms “promoting plant growth” and “stimulating plant growth” are used interchangeably herein, and refer to the ability to enhance or increase at least one of the plant's height, weight, leaf size, root size, or stem size, to increase protein yield from the plant or to increase grain yield of the plant.

A “protein or peptide that protects a plant from a pathogen” as used herein includes any protein or peptide that makes a plant exposed to the protein or peptide less susceptible to infection with a pathogen.

A “protein or peptide that enhances stress resistance in a plant” as used herein includes any protein or peptide that makes a plant exposed to the protein or peptide more resistant to stress.

The term “plant binding protein or peptide” refers to any peptide or protein capable of specifically or non-specifically binding to any part of a plant (e.g., roots or arial portions of a plant such as leaves foliage, stems, flowers, or fruits) or to plant matter.

The term “targeting sequence” as used herein refers to a polypeptide sequence that results in the localization of a longer polypeptide or the protein to the exosporium of a Bacillus cereus family member.

### Recombinant Exosporium-Producing Bacillus Cells Expressing Fusion Proteins

The fusion proteins contain a targeting sequence, an exosporium protein, or an exosporium protein fragment that targets the fusion protein to the exosporium of a Bacillus cereus family member and: (a) a plant growth stimulating protein or peptide; (b) a protein or peptide that protects a plant from a pathogen; (c) a protein or peptide that enhances stress resistance of a plant; (d) a plant binding protein or peptide; or (e) a plant immune system enhancer protein or peptide. When expressed in Bacillus cereus family member bacteria, these fusion proteins are targeted to the exosporium layer of the spore and are physically oriented such that the protein or peptide is displayed on the outside of the spore.

This Bacillus exosporium display (BEMD) system can be used to deliver peptides, enzymes, and other proteins to plants (e.g., to plant foliage, fruits, flowers, stems, or roots) or to a plant growth medium such as soil. Peptides, enzymes, and proteins delivered to the soil or another plant growth medium in this manner persist and exhibit activity in the soil for extended periods of time. Introduction of recombinant exosporium-producing Bacillus cells expressing the fusion proteins described herein into soil or the rhizosphere of a plant leads to a beneficial enhancement of plant growth in many different soil conditions. The use of the BEMD to create these enzymes allows them to continue to exert their beneficial results to the plant and the rhizosphere over the first months of a plants life.

### Targeting Sequences, Exosporium Proteins, and Exosporium Protein Fragments

For ease of reference, the SEQ ID NOS. for the peptide and protein sequences referred to herein are listed in Table 1 below.

TABLE 1
Peptide and Protein Sequences

| Protein, Protein Fragment, or Targeting Sequence | Sequence Identification Number |
|---|---|
| AA 1-41 of BclA | SEQ ID NO: 1* |
| (B. anthracis Sterne) | |
| Full length BclA | SEQ ID NO: 2* |
| AA 1-33 of | SEQ ID NO: 3 |
| BetA/BAS3290 | |
| (B. anthracis Sterne) | |
| Full length BetA/BAS3290 | SEQ ID NO: 4 |
| Met + AA 2-43 of | SEQ ID NO: 5 |
| BAS4623 | |
| (B. anthracis Sterne) | |
| Full length BAS4623 | SEQ ID NO: 6 |
| AA 1-34 of BclB | SEQ ID NO: 7 |
| (B. anthracis Sterne) | |
| Full length BclB | SEQ ID NO: 8 |
| AA 1-30 of BAS1882 (B. anthracis Sterne) | SEQ ID NO: 9 |
| Full length BAS1882 | SEQ ID NO: 10 |
| AA 1-39 of gene 2280 (B. weihenstephensis KBAB4) | SEQ ID NO: 11 |
| Full length KBAB4 gene 2280 | SEQ ID NO: 12 |
| AA 1-39 of gene 3572 (B. weihenstephensis KBAB4) | SEQ ID NO: 13 |
| Full Length KBAB4 gene 3572 | SEQ ID NO: 14 |
| AA 1-49 of Exosporium Leader Peptide | SEQ ID NO: 15 |
| (B. cereus VD200) | |
| Full Length Exosporium Leader Peptide | SEQ ID NO: 16 |
| AA 1-33 of Exosporium Leader Peptide | SEQ ID NO: 17 |
| (B. cereus VD166) | |
| Full Length Exosporium Leader Peptide | SEQ ID NO: 18 |
| AA 1-39 of hypothetical protein IKG_04663 | SEQ ID NO: 19 |
| (B. cereus VD200) | |
| Full Length hypothetical protein IKG_04663, partial | SEQ ID NO: 20 |
| AA 1-39 of YVTN β-propeller protein | SEQ ID NO: 21 |
| (B. weihenstephensis KBAB4) | |
| Full length YVTN β-propeller protein KBAB4 | SEQ ID NO: 22 |
| AA 1-30 of hypothetical protein bcerkbab4_2363 | SEQ ID NO: 23 |
| (B. weihenstephensis KBAB4) | |
| Full length hypothetical protein bcerkbab4_2363 | SEQ ID NO: 24 |
| KBAB4 | |
| AA 1-30 of hypothetical protein bcerkbab4_2131 | SEQ ID NO: 25 |
| (B. weihenstephensis KBAB4) | |
| Full length hypothetical protein bcerkbab4_2131 | SEQ ID NO: 26 |
| AA 1-36 of triple helix repeat containing collagen | SEQ ID NO: 27 |
| (B. weihenstephensis KBAB4) | |
| Full length triple helix repeat-containing collagen KBAB4 | SEQ ID NO: 28 |
| AA 1-39 of hypothetical protein bmyco0001_21660 | SEQ ID NO: 29 |
| (B. mycoides 2048) | |
| Full length hypothetical protein bmyco0001_21660 | SEQ ID NO: 30 |
| AA 1-30 of hypothetical protein bmyc0001_22540 | SEQ ID NO: 31 |
| (B. mycoides 2048) | |
| Full length hypothetical protein bmyc0001_22540 | SEQ ID NO: 32 |
| AA 1-21 of hypothetical protein bmyc0001_21510 | SEQ ID NO: 33 |
| (B. mycoides 2048) | |
| Full length hypothetical protein bmyc0001_21510 | SEQ ID NO: 34 |
| AA 1-22 of collagen triple helix repeat protein | SEQ ID NO: 35 |
| (B. thuringiensis 35646) | |
| Full length collagen triple helix repeat protein | SEQ ID NO: 36 |
| AA 1-35 of hypothetical protein WP_69652 | SEQ ID NO: 43 |
| (B. cereus) | |
| Full length hypothetical protein WP_69652 | SEQ ID NO: 44 |
| AA 1-41 of exosporium leader WP016117717 | SEQ ID NO: 45 |
| (B. cereus) | |
| Full length exosporium leader WP016117717 | SEQ ID NO: 46 |
| AA 1-49 of exosporium peptide WP002105192 | SEQ ID NO: 47 |
| (B. cereus) | |
| Full length exosporium peptide WP002105192 | SEQ ID NO: 48 |
| AA 1-38 of hypothetical protein WP87353 | SEQ ID NO: 49 |
| (B. cereus) | |
| Full length hypothetical protein WP87353 | SEQ ID NO: 50 |
| AA 1-39 of exosporium peptide 02112369 | SEQ ID NO: 51 |
| (B. cereus) | |
| Full length exosporium peptide 02112369 | SEQ ID NO: 52 |
| AA 1-39 of exosporium protein WP016099770 | SEQ ID NO: 53 |
| (B. cereus) | |
| Full length exosporium protein WP016099770 | SEQ ID NO: 54 |
| AA 1-36 of hypothetical protein YP006612525 | SEQ ID NO: 55 |
| (B. thuringiensis) | |
| Full length hypothetical protein YP006612525 | SEQ ID NO: 56 |
| AA 1-136 of hypothetical protein TIGR03720 | SEQ ID NO: 57** |
| (B. mycoides) | |
| Full length hypothetical protein TIGR03720 | SEQ ID NO: 58** |
| AA 1-196 of BclA | SEQ ID NO: 59* |
| (B. anthracis Sterne) | |
| Met + AA 20-35 of BclA | SEQ ID NO: 60 |
| (B. anthracis Sterne) | |
| Met + AA 12-27 of BetA/BAS3290 | SEQ ID NO: 61 |
| (B. anthracis Sterne) | |
| Met + AA 18-33 of gene 2280 | SEQ ID NO: 62 |
| (B. weihenstephensis KBAB4) | |
| Met + AA 18-33 of gene 3572 | SEQ ID NO: 63 |
| (B. weihenstephensis KBAB4) | |
| Met + AA 12-27 of Exosporium Leader Peptide | SEQ ID NO: 64 |
| (B. cereus VD166) | |
| Met + AA 18-33 of YVTN β-propeller protein | SEQ ID NO: 65 |
| (B. weihenstephensis KBAB4) | |
| Met + AA 9-24 of hypothetical protein bcerkbab4_2363 | SEQ ID NO: 66 |
| (B. weihenstephensis KBAB4) | |
| Met + AA 9-24 of hypothetical protein bcerkbab4_2131 | SEQ ID NO: 67 |
| (B. weihenstephensis KBAB4) | |
| Met + AA 9-24 of hypothetical protein bmyc0001_22540 | SEQ ID NO: 68 |
| (B. mycoides 2048) | |
| Met + AA 9-24 of | SEQ ID NO: 69 |
| BAS1882 | |
| (B. anthracis Sterne) | |
| Met + AA 20-35 of exosporium leader WP016117717 | SEQ ID NO: 70 |
| (B. cereus) | |
| Full length InhA | SEQ ID NO: 71 |
| (B. mycoides) | |
| Full length BAS1141 (ExsY) | SEQ ID NO: 72 |
| (B. anthracis Sterne) | |
| Full length BAS1144 (BxpB/ExsFA) | SEQ ID NO: 73 |
| (B. anthracis Sterne) | |
| Full length BAS1145 (CotY) | SEQ ID NO: 74 |
| (B. anthracis Sterne) | |
| Full length BAS1140 | SEQ ID NO: 75 |
| (B. anthracis Sterne) | |
| Full length ExsFB | SEQ ID NO: 76 |
| (B. anthracis H9401) | |
| Full length InhA1 | SEQ ID NO: 77 |
| (B. thuringiensis HD74) | |
| Full length ExsJ | SEQ ID NO: 78 |
| (B. cereus ATCC 10876) | |
| Full length ExsH | SEQ ID NO: 79 |
| (B. cereus) | |
| Full length YjcA | SEQ ID NO: 80 |
| (B. anthracis Ames) | |
| Full length YjcB | SEQ ID NO: 81 |
| (B. anthracis) | |
| Full length BclC | SEQ ID NO: 82 |
| (B. anthracis Sterne) | |
| Full length acid phosphatase | SEQ ID NO: 83 |
| (Bacillus thuringiensis serovar konkukian str. 97-27) | |
| Full length InhA2 | SEQ ID NO: 84 |
| (B. thuringiensis HD74) | |

AA = amino acids
*B. anthracis Sterne strain BclA has 100% sequence identity with B. thuringiensis BclA. Thus, SEQ ID NOS: 1, 2, and 59 also represent amino acids 1-41 of B. thuringiensis BclA, full length B. thuringiensis BclA, and amino acids 1-196 of B. thuringiensis BclA, respectively. Likewise, SEQ ID NO: 60 also represents a methionine residue plus amino acids 20-35 of B. thuringiensis BclA.
**B. mycoides hypothetical protein TIGR03720 has 100% sequence identity with B. mycoides hypothetical protein WP003189234. Thus, SEQ ID NOs: 57 and 58 also represent amino acids 1-136 of B. mycoides hypothetical protein WP003189234 and full length B. mycoides hypothetical protein WP003189234, respectively.

Bacillus is a genus of rod-shaped bacteria. The Bacillus cereus family of bacteria includes the species Bacillus anthracis, Bacillus cereus, Bacillus thuringiensis, Bacillus mycoides, Bacillus pseudomycoides, Bacillus samanii, Bacillus gaemokensis, Bacillus toyoiensis, and Bacillus weihenstephensis. Under stressful environmental conditions, Bacillus cereus family bacteria undergo sporulation and form oval endospores that can stay dormant for extended periods of time. The outermost layer of the endospores is known as the exosporium and comprises a basal layer surrounded by an external nap of hair-like projections. Filaments on the hair-like nap are predominantly formed by the collagen-like glycoprotein BclA, while the basal layer is comprised of a number of different proteins. Another collagen-related protein, BclB, is also present in the exosporium and exposed on endospores of Bacillus cereus family members.

BclA, the major constituent of the surface nap, has been shown to be attached to the exosporium with its amino-terminus (N-terminus) positioned at the basal layer and its carboxy-terminus (C-terminus) extending outward from the spore.

It was previously discovered that certain sequences from the N-terminal regions of BclA and BclB could be used to target a peptide or protein to the exosporium of a Bacillus cereus endospore (see U.S. Patent Publication Nos. 2010/0233124 and 2011/0281316, and Thompson, et al., “Targeting of the BclA and BclB Proteins to the Bacillus anthracis Spore Surface,” Molecular Microbiology, 70(2):421-34 (2008), the entirety of each of which is hereby incorporated by reference). It was also found that the BetA/BAS3290 protein of Bacillus anthracis localized to the exosporium.

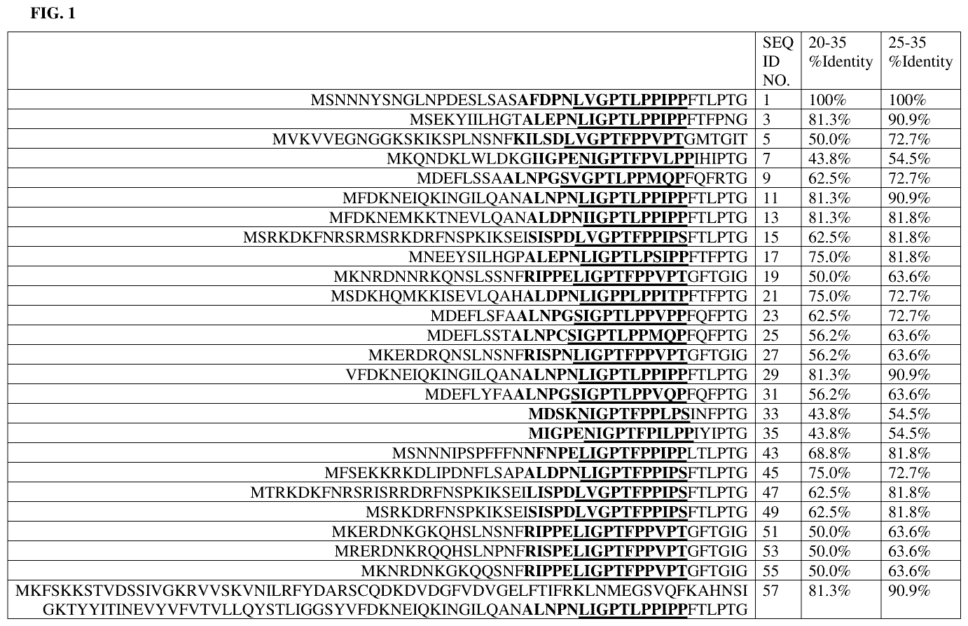

In particular, amino acids 20-35 of BclA from Bacillus anthracis Sterne strain have been found to be sufficient for targeting to the exosporium. A sequence alignment of amino acids 1-41 of BclA (SEQ ID NO: 1) with the corresponding N-terminal regions of several other Bacillus cereus family exosporium proteins and Bacillus cereus family proteins having related sequences is shown in FIG. 1. As can be seen from FIG. 1, there is a region of high-homology among all of the proteins in the region corresponding to amino acids 20-41 of BclA. However, in these sequences, the amino acids corresponding to amino acids 36-41 of BclA contain secondary structure and are not necessary for fusion protein localization to the exosporium. The conserved targeting sequence region of BclA (amino acids 20-35 of SEQ ID NO: 1) is shown in bold in FIG. 1 and corresponds to the minimal targeting sequence needed for localization to the exosporium. A more highly conserved region spanning amino acids 25-35 of BclA within the targeting sequence is underlined in the sequences in FIG. 1, and is the recognition sequence for ExsFA/BxpB/ExsFB and homologs, which direct and assemble the described proteins on the surface of the exosporium The amino acid sequences of SEQ ID NOS. 3, 5, and 7 in FIG. 1 are amino acids 1-33 of Bacillus anthracis Sterne strain BetA/BAS3290, a methionine followed by amino acids 2-43 of Bacillus anthracis Sterne strain BAS4623, and amino acids 1-34 of Bacillus anthracis Sterne strain BclB, respectively. (For BAS4623, it was found that replacing the valine present at position 1 in the native protein with a methionine resulted in better expression.) As can be seen from FIG. 1, each of these sequences contains a conserved region corresponding to amino acids 20-35 of BclA (SEQ ID NO: 1; shown in bold), and a more highly conserved region corresponding to amino acids 20-35 of BclA (underlined).

Additional proteins from Bacillus cereus family members also contain the conserved targeting region. In particular, in FIG. 1, SEQ ID NO: 9 is amino acids 1-30 of Bacillus anthracis Sterne strain BAS1882, SEQ ID NO: 11 is amino acids 1-39 of the Bacillus weihenstephensis KBAB4 2280 gene product, SEQ ID NO: 13 is amino acids 1-39 of the Bacillus weihenstephensis KBAB4 3572 gene product, SEQ ID NO: 15 is amino acids 1-49 of Bacillus cereus VD200 exosporium leader peptide, SEQ ID NO: 17 is amino acids 1-33 of Bacillus cereus VD166 exosporium leader peptide, SEQ ID NO: 19 is amino acids 1-39 of Bacillus cereus VD200 hypothetical protein IKG_04663, SEQ ID NO: 21 is amino acids 1-39 of Bacillus weihenstephensis KBAB4 YVTN B-propeller protein, SEQ ID NO: 23 is amino acids 1-30 of Bacillus weihenstephensis KBAB4 hypothetical protein bcerkbab4_2363, SEQ ID NO: 25 is amino acids 1-30 of Bacillus weihenstephensis KBAB4 hypothetical protein berkbab4_2131, SEQ ID NO: 27 is amino acids 1-36 of Bacillus weihenstephensis KBAB4 triple helix repeat containing collagen, SEQ ID NO: 29 is amino acids 1-39 of Bacillus mycoides 2048 hypothetical protein bmyco0001_21660, SEQ ID NO: 31 is amino acids 1-30 of Bacillus mycoides 2048 hypothetical protein bmyc0001_22540, SEQ ID NO: 33 is amino acids 1-21 of Bacillus mycoides 2048 hypothetical protein bmyc0001_21510, SEQ ID NO: 35 is amino acids 1-22 of Bacillus thuringiensis 35646 collagen triple helix repeat protein, SEQ ID NO: 43 is amino acids 1-35 of Bacillus cereus hypothetical protein WP_69652, SEQ ID NO: 45 is amino acids 1-41 of Bacillus cereus exosporium leader WP016117717, SEQ ID NO: 47 is amino acids 1-49 of Bacillus cereus exosporium peptide WP002105192, SEQ ID NO: 49 is amino acids 1-38 of Bacillus cereus hypothetical protein WP87353, SEQ ID NO: 51 is amino acids 1-39 of Bacillus cereus exosporium peptide 02112369, SEQ ID NO: 53 is amino acids 1-39 of Bacillus cereus exosporium protein WP016099770, SEQ ID NO: 55 is amino acids 1-36 of Bacillus thuringiensis hypothetical protein YP006612525, and SEQ ID NO: 57 is amino acids 1-136 of Bacillus mycoides hypothetical protein TIGR03720. As shown in FIG. 1, each of the N-terminal regions of these proteins contains a region that is conserved with amino acids 20-35 of BclA (SEQ ID NO: 1), and a more highly conserved region corresponding to amino acids 25-35 of BclA.

Any portion of BclA which includes amino acids 20-35 can be used as the targeting sequence. In addition, full-length exosporium proteins or exosporium protein fragments can be used for targeting the fusion proteins to the exosporium. Thus, full-length BclA or a fragment of BclA that includes amino acids 20-35 can be used for targeting to the exosporium. For example, full length BclA (SEQ ID NO: 2) or a midsized fragment of BclA that lacks the carboxy-terminus such as SEQ ID NO: 59 (amino acids 1-196 of BclA) can be used to target the fusion proteins to the exosporium. Midsized fragments such as the fragment of SEQ ID NO: 59 have less secondary structure than full length BclA and have been found to be suitable for use as a targeting sequence. The targeting sequence can also comprise much shorter portions of BclA which include amino acids 20-35, such as SEQ ID NO: 1 (amino acids 1-41 of BclA), amino acids 1-35 of SEQ ID NO: 1, amino acids 20-35 of SEQ ID NO: 1, or SEQ ID NO: 60 (a methionine residue linked to amino acids 20-35 of BclA). Even shorter fragments of BclA which include only some of amino acids 20-35 also exhibit the ability to target fusion proteins to the exosporium. For example, the targeting sequence can comprise amino acids 22-31 of SEQ ID NO: 1, amino acids 22-33 of SEQ ID NO: 1, or amino acids 20-31 of SEQ ID NO: 1.

Alternatively, any portion of BctA/BAS3290, BAS4623, BclB, BAS1882, the KBAB4 2280 gene product, the KBAB4 3572 gene product, B. cereus VD200 exosporium leader peptide, B. cereus VD166 exosporium leader peptide, B. cereus VD200 hypothetical protein IKG_04663, B. weihenstephensis KBAB4 YVTN B-propeller protein, B. weihenstephensis KBAB4 hypothetical protein bcerkbab4_2363, B. weihenstephensis KBAB4 hypothetical protein bcerkbab4_2131, B. weihenstephensis KBAB4 triple helix repeat containing collagen, B. mycoides 2048 hypothetical protein bmyco0001_21660, B. mycoides 2048 hypothetical protein bmyc0001_22540, B. mycoides 2048 hypothetical protein bmyc0001_21510, B. thuringiensis 35646 collagen triple helix repeat protein, B. cereus hypothetical protein WP_69652, B. cereus exosporium leader WP016117717, B. cereus exosporium peptide WP002105192, B. cereus hypothetical protein WP87353, B. cereus exosporium peptide 02112369, B. cereus exosporium protein WP016099770, B. thuringiensis hypothetical protein YP006612525, or B. mycoides hypothetical protein TIGR03720 which includes the amino acids corresponding to amino acids 20-35 of BclA can serve as the targeting sequence. As can be seen from FIG. 1, amino acids 12-27 of BetA/BAS3290, amino acids 23-38 of BAS4623, amino acids 13-28 of BclB, amino acids 9-24 of BAS1882, amino acids 18-33 of KBAB4 2280 gene product, amino acids 18-33 of KBAB4 3572 gene product, amino acids 28-43 of B. cereus VD200 exosporium leader peptide, amino acids 12-27 of B. cereus VD166 exosporium leader peptide, amino acids 18-33 of B. cereus VD200 hypothetical protein IKG_04663, amino acids 18-33 B. weihenstephensis KBAB4 YVTN β-propeller protein, amino acids 9-24 of B. weihenstephensis KBAB4 hypothetical protein bcerkbab4_2363, amino acids 9-24 of B. weihenstephensis KBAB4 hypothetical protein bcerkbab4_2131, amino acids 15-30 of B. weihenstephensis KBAB4 triple helix repeat containing collagen, amino acids 18-33 of B. mycoides 2048 hypothetical protein bmyco0001_21660, amino acids 9-24 of B. mycoides 2048 hypothetical protein bmyc0001_22540, amino acids 1-15 of B. mycoides 2048 hypothetical protein bmyc0001_21510, amino acids 1-16 of B. thuringiensis 35646 collagen triple helix repeat protein, amino acids 14-29 of B. cereus hypothetical protein WP_69652, amino acids 20-35 of B. cereus exosporium leader WP016117717, amino acids 28-43 of B. cereus exosporium peptide WP002105192, amino acids 17-32 of B. cereus hypothetical protein WP87353, amino acids 18-33 of B. cereus exosporium peptide 02112369, amino acids 18-33 of B. cereus exosporium protein WP016099770, amino acids 15-30 of B. thuringiensis hypothetical protein YP006612525, and amino acids 115-130 of B. mycoides hypothetical protein TIGR03720 correspond to amino acids 20-35 of BclA. Thus, any portion of these proteins that includes the above-listed corresponding amino acids can serve as the targeting sequence.

Furthermore, any amino acid sequence comprising amino acids 20-35 of BclA, or any of the above-listed corresponding amino acids can serve as the targeting sequence.

Thus, the targeting sequence can comprise amino acids 1-35 of SEQ ID NO: 1, amino acids 20-35 of SEQ ID NO: 1, SEQ ID NO: 1, SEQ ID NO: 60, amino acids 22-31 of SEQ ID NO: 1, amino acids 22-33 of SEQ ID NO: 1, or amino acids 20-31 of SEQ ID NO: 1. Alternatively, the targeting sequence consists of amino acids 1-35 of SEQ ID NO: 1, amino acids 20-35 of SEQ ID NO: 1, SEQ ID NO: 1, or SEQ ID NO: 60. Alternatively, the targeting sequence can consist of amino acids 22-31 of SEQ ID NO: 1, amino acids 22-33 of SEQ ID NO: 1, or amino acids 20-31 of SEQ ID NO: 1. Alternatively, the exosporium protein can comprise full length BclA (SEQ ID NO: 2), or the exosporium protein fragment can comprise a midsized fragment of BclA that lacks the carboxy-terminus, such as SEQ ID NO: 59 (amino acids 1-196 of BclA). Alternatively, the exosporium protein fragment can consist of SEQ ID NO: 59.

The targeting sequence can also comprise amino acids 1-27 of SEQ ID NO: 3, amino acids 12-27 of SEQ ID NO: 3, or SEQ ID NO: 3, or the exosporium protein can comprise full length BetA/BAS3290 (SEQ ID NO: 4). It has also been found that a methionine residue linked to amino acids 12-27 of BetA/BAS3290 can be used as a targeting sequence. Thus, the targeting sequence can comprise SEQ ID NO: 61. The targeting sequence can also comprise amino acids 14-23 of SEQ ID NO: 3, amino acids 14-25 of SEQ ID NO: 3, or amino acids 12-23 of SEQ ID NO: 3.

The targeting sequence can also comprise amino acids 1-38 of SEQ ID NO: 5, amino acids 23-38 of SEQ ID NO: 5, or SEQ ID NO: 5, or the exosporium protein can comprise full length BAS4623 (SEQ ID NO: 6).

Alternatively, the targeting sequence can comprise amino acids 1-28 of SEQ ID NO: 7, amino acids 13-28 of SEQ ID NO: 7, or SEQ ID NO: 7, or the exosporium protein can comprise full length BclB (SEQ ID NO: 8).

The targeting sequence can also comprise amino acids 1-24 of SEQ ID NO: 9, amino acids 9-24 of SEQ ID NO: 9, or SEQ ID NO: 9, or the exosporium protein can comprise full length BAS1882 (SEQ ID NO: 10). A methionine residue linked to amino acids 9-24 of BAS1882 can also be used as a targeting sequence. Thus, the targeting sequence can comprise SEQ ID NO: 69.

The targeting sequence can also comprise amino acids 1-33 of SEQ ID NO: 11, amino acids 18-33 of SEQ ID NO: 11, or SEQ ID NO: 11, or the exosporium protein can comprise the full length B. weihenstephensis KBAB4 2280 gene product (SEQ ID NO: 12). A methionine residue linked to amino acids 18-33 of the B. weihenstephensis KBAB4 2280 gene product can also be used as a targeting sequence. Thus, the targeting sequence can comprise SEQ ID NO: 62.

The targeting sequence can also comprise amino acids 1-33 of SEQ ID NO: 13, amino acids 18-33 of SEQ ID NO: 13, or SEQ ID NO: 13, or the exosporium protein can comprise the full length B. weihenstephensis KBAB4 3572 gene product (SEQ ID NO:14). A methionine residue linked to amino acids 18-33 of the B. weihenstephensis KBAB4 3572 gene product can also be used as a targeting sequence. Thus, the targeting sequence can comprise SEQ ID NO: 63.

Alternatively, the targeting sequence can comprise amino acids 1-43 of SEQ ID NO: 15, amino acids 28-43 of SEQ ID NO: 15, or SEQ ID NO: 15, or the exosporium protein can comprise full length B. cereus VD200 exosporium leader peptide (SEQ ID NO: 16).

The targeting sequence can also comprise amino acids 1-27 of SEQ ID NO: 17, amino acids 12-27 of SEQ ID NO: 17, or SEQ ID NO: 17, or the exosporium protein can comprise full-length B. cereus VD166 exosporium leader peptide (SEQ ID NO: 18). A methionine residue linked to amino acids 12-27 of the B. cereus VD166 exosporium leader peptide can also be used as a targeting sequence. Thus, the targeting sequence can comprise SEQ ID NO: 64.

The targeting sequence can also comprise amino acids 1-33 of SEQ ID NO: 19, amino acids 18-33 of SEQ ID NO: 19, or SEQ ID NO: 19, or the exosporium protein can comprise full length B. cereus VD200 hypothetical protein IKG_04663 (SEQ ID NO:20).

Alternatively, the targeting sequence comprises amino acids 1-33 of SEQ ID NO: 21, amino acids 18-33 of SEQ ID NO: 21, or SEQ ID NO: 21, or the exosporium protein can comprise full length B. weihenstephensis KBAB4 YVTN B-propeller protein (SEQ ID NO: 22). A methionine residue linked to amino acids 18-33 of the B. weihenstephensis KBAB4 YVTN B-propeller protein can also be used as a targeting sequence. Thus, the targeting sequence can comprise SEQ ID NO: 65.

The targeting sequence can also comprise amino acids 1-24 of SEQ ID NO: 23, amino acids 9-24 of SEQ ID NO: 23, or SEQ ID NO: 23, or the exosporium protein can comprise full length B. weihenstephensis KBAB4 hypothetical protein bcerkbab4_2363 (SEQ ID NO: 24). A methionine residue linked to amino acids 9-24 of B. weihenstephensis KBAB4 hypothetical protein bcerkbab4_2363 can also be used as a targeting sequence. Thus, the targeting sequence can comprise SEQ ID NO: 66.

The targeting sequence comprise amino acids 1-24 of SEQ ID NO: 25, amino acids 9-24 of SEQ ID NO: 25, or SEQ ID NO: 25, or the exosporium protein can comprise full length B. weihenstephensis KBAB4 hypothetical protein bcerkbab4_2131 (SEQ ID NO: 26). A methionine residue linked to amino acids 9-24 of B. weihenstephensis KBAB4 hypothetical protein bcerkbab4_2131 can also be used as a targeting sequence. Thus, the targeting sequence can comprise SEQ ID NO: 67.

Alternatively, the targeting sequence comprises amino acids 1-30 of SEQ ID NO: 27, amino acids 15-30 of SEQ ID NO: 27, or SEQ ID NO:27, or the exosporium protein can comprise full length B. weihenstephensis KBAB4 triple helix repeat containing collagen (SEQ ID NO: 28).

The targeting sequence can also comprise amino acids 1-33 of SEQ ID NO: 29, amino acids 18-33 of SEQ ID NO: 29, or SEQ ID NO: 29, or the exosporium protein can comprise full length B. mycoides 2048 hypothetical protein bmyco0001_21660 (SEQ ID NO: 30).

The targeting sequence can also comprise amino acids 1-24 of SEQ ID NO: 31, amino acids 9-24 of SEQ ID NO: 31, or SEQ ID NO: 31, or the exosporium protein can comprise full length B. mycoides 2048 hypothetical protein bmyc0001_22540 (SEQ ID NO: 32). A methionine residue linked to amino acids 9-24 of B. mycoides 2048 hypothetical protein bmyc0001_22540 can also be used as a targeting sequence. Thus, the targeting sequence can comprise SEQ ID NO: 68.

Alternatively, the targeting sequence comprises amino acids 1-15 of SEQ ID NO: 33, SEQ ID NO: 33, or the exosporium protein comprises full length B. mycoides 2048 hypothetical protein bmyc0001_21510 (SEQ ID NO:34).

The targeting sequence can also comprise amino acids 1-16 of SEQ ID NO: 35, SEQ ID NO: 35, or the exosporium protein can comprise full length B. thuringiensis 35646 collagen triple helix repeat protein (SEQ ID NO: 36).

The targeting sequence can comprise amino acids 1-29 of SEQ ID NO: 43, amino acids 14-29 of SEQ ID NO: 43, or SEQ ID NO: 43, or the exosporium protein can comprise full length B. cereus hypothetical protein WP_69652 (SEQ ID NO: 44).

Alternatively, the targeting sequence can comprise amino acids 1-35 of SEQ ID NO: 45, amino acids 20-35 of SEQ ID NO: 45, or SEQ ID NO: 45, or the exosporium protein can comprise full length B. cereus exosporium leader WP016117717 (SEQ ID NO: 46). A methionine residue linked to amino acids 20-35 of B. cereus exosporium leader WP016117717 can also be used as a targeting sequence. Thus, the targeting sequence can comprise SEQ ID NO: 70.

The targeting sequence can comprise amino acids 1-43 of SEQ ID NO: 47, amino acids 28-43 of SEQ ID NO: 47, or SEQ ID NO: 47, or the exosporium protein can comprise full length B. cereus exosporium peptide WP002105192 (SEQ ID NO: 48).

The targeting sequence can comprise amino acids 1-32 of SEQ ID NO: 49, amino acids 17-32 of SEQ ID NO: 49, or SEQ ID NO: 49, or the exosporium protein can comprise full length B. cereus hypothetical protein WP87353 (SEQ ID NO: 50).

Alternatively, the targeting sequence can comprise amino acids 1-33 of SEQ ID NO: 51, amino acids 18-33 of SEQ ID NO: 51, or SEQ ID NO: 51, or the exosporium protein can comprise full length B. cereus exosporium peptide 02112369 (SEQ ID NO: 52).

The targeting sequence can comprise amino acids 1-33 of SEQ ID NO: 53, amino acids 18-33 of SEQ ID NO: 53, or SEQ ID NO: 53, or the exosporium protein can comprise full length B. cereus exosporium protein WP016099770 (SEQ ID NO: 54).

Alternatively, the targeting sequence can comprise acids 1-30 of SEQ ID NO: 55, amino acids 15-30 of SEQ ID NO: 55, or SEQ ID NO: 55, or the exosporium protein can comprise full length B. thuringiensis hypothetical protein YP006612525 (SEQ ID NO: 56).

The targeting sequence can also comprise amino acids 1-130 of SEQ ID NO: 57, amino acids 115-130 of SEQ ID NO: 57, or SEQ ID NO: 57, or the exosporium protein can comprise full length B. mycoides hypothetical protein TIGR03720 (SEQ ID NO: 58).

In addition, it can readily be seen from the sequence alignment in FIG. 1 that while amino acids 20-35 of BclA are conserved, and amino acids 25-35 are more conserved, some degree of variation can occur in this region without affecting the ability of the targeting sequence to target a protein to the exosporium. FIG. 1 lists the percent identity of each of corresponding amino acids of each sequence to amino acids 20-35 of BclA (“20-35% Identity”) and to amino acids 25-35 of BclA (“25-35% Identity”). Thus, for example, as compared to amino acids 20-35 of BclA, the corresponding amino acids of BetA/BAS3290 are about 81.3% identical, the corresponding amino acids of BAS4623 are about 50.0% identical, the corresponding amino acids of BclB are about 43.8% identical, the corresponding amino acids of BAS1882 are about 62.5% identical, the corresponding amino acids of the KBAB4 2280 gene product are about 81.3% identical, and the corresponding amino acids of the KBAB4 3572 gene product are about 81.3% identical. The sequence identities over this region for the remaining sequences are listed in FIG. 1.

With respect to amino acids 25-35 of BclA, the corresponding amino acids of BetA/BAS3290 are about 90.9% identical, the corresponding amino acids of BAS4623 are about 72.7% identical, the corresponding amino acids of BclB are about 54.5% identical, the corresponding amino acids of BAS1882 are about 72.7% identical, the corresponding amino acids of the KBAB4 2280 gene product are about 90.9% identical, and the corresponding amino acids of the KBAB4 3572 gene product are about 81.8% identical. The sequence identities over this region for the remaining sequences are listed in FIG. 1.

Thus, the targeting sequence can comprise an amino acid sequence having at least about 43% identity with amino acids 20-35 of SEQ ID NO: 1, wherein the identity with amino acids 25-35 is at least about 54%. Alternatively, the targeting sequence consists of an amino acid sequence consisting of 16 amino acids and having at least about 43% identity with amino acids 20-35 of SEQ ID NO: 1, wherein the identity with amino acids 25-35 is at least about 54%.

The targeting sequence can also comprise an amino acid sequence having at least about 50% identity with amino acids 20-35 of SEQ ID NO: 1, wherein the identity with amino acids 25-35 is at least about 63%. Alternatively the targeting sequence consists of an amino acid sequence consisting of 16 amino acids and having at least about 50% identity with amino acids 20-35 of SEQ ID NO: 1, wherein the identity with amino acids 25-35 is at least about 63%.

The targeting sequence can also comprise an amino acid sequence having at least about 50% identity with amino acids 20-35 of SEQ ID NO: 1, wherein the identity with amino acids 25-35 is at least about 72%. Alternatively, the targeting sequence consists of an amino acid sequence consisting of 16 amino acids and having at least about 50% identity with amino acids 20-35 of SEQ ID NO: 1, wherein the identity with amino acids 25-35 is at least about 72%.

The targeting sequence can also comprise an amino acid sequence having at least about 56% identity with amino acids 20-35 of SEQ ID NO: 1, wherein the identity with amino acids 25-35 is at least about 63%. Alternatively, the targeting sequence consists of an amino acid sequence consisting of 16 amino acids and having at least about 56% identity with amino acids 20-35 of SEQ ID NO: 1, wherein the identity with amino acids 25-35 is at least about 63%.

Alternatively, the targeting sequence can comprise an amino sequence having at least about 62% identity with amino acids 20-35 of SEQ ID NO: 1, wherein the identity with amino acids 25-35 is at least about 72%. The targeting sequence can also consist of an amino acid sequence consisting of 16 amino acids and having at least about 62% identity with amino acids 20-35 of SEQ ID NO: 1, wherein the identity with amino acids 25-35 of SEQ ID NO: 1 is at least about 72%.

The targeting sequence can comprise an amino acid sequence having at least 68% identity with amino acids 20-35 of SEQ ID NO: 1, wherein the identity with amino acids 25-35 is at least about 81%. Alternatively, the targeting sequence consists of an amino acid sequence consisting of 16 amino acids and having at least 68% identity with amino acids 20-35 of SEQ ID NO: 1, wherein the identity with amino acids 25-35 is at least about 81%.

The targeting sequence can also comprises an amino sequence having at least about 75% identity with amino acids 20-35 of SEQ ID NO: 1, wherein the identity with amino acids 25-35 is at least about 72%. Alternatively, the targeting sequence consists of an amino acid sequence consisting of 16 amino acids and having at least about 75% identity with amino acids 20-35 of SEQ ID NO: 1, wherein the identity with amino acids 25-35 of SEQ ID NO: 1 is at least about 72%.

The targeting sequence can also comprise an amino sequence having at least about 75% identity with amino acids 20-35 of SEQ ID NO: 1, wherein the identity with amino acids 25-35 is at least about 81%. Alternatively, the targeting sequence consists of an amino acid sequence consisting of 16 amino acids and having at least about 75% identity with amino acids 20-35 of SEQ ID NO: 1, wherein the identity with amino acids 25-35 of SEQ ID NO:1 is at least about 81%.

The targeting sequence can also comprise an amino acid sequence having at least about 81% identity with amino acids 20-35 of SEQ ID NO: 1, wherein the identity with amino acids 25-35 is at least about 81%. Alternatively, the targeting sequence consists of an amino acid sequence consisting of 16 amino acids and having at least about 81% identity with amino acids 20-35 of SEQ ID NO: 1, wherein the identity with amino acids 25-35 is at least about 81%.

The targeting sequence can comprise an amino acid sequence having at least about 81% identity with amino acids 20-35 of SEQ ID NO: 1, wherein the identity with amino acids 25-35 is at least about 90%. Alternatively, the targeting sequence consists of an amino acid sequence consisting of 16 amino acids and having at least about 81% identity with amino acids 20-35 of SEQ ID NO: 1, wherein the identity with amino acids 25-35 is at least about 90%.

The skilled person will recognize that variants of the above sequences can also be used as targeting sequences, so long as the targeting sequence comprises amino acids 20-35 of BclA, the corresponding amino acids of BetA/BAS3290, BAS4263, BclB, BAS1882, the KBAB4 2280 gene product, or the KBAB 3572 gene product, or a sequence comprising any of the above noted sequence identities to amino acids 20-35 and 25-35 of BclA is present.

It has further been discovered that certain Bacillus cereus family exosporium proteins which lack regions having homology to amino acids 25-35 of BclA can also be used to target a peptide or protein to the exosporium of a Bacillus cereus family member. In particular, the fusion proteins can comprise an exosporium protein comprising SEQ ID NO: 71 (B. mycoides InhA), an exosporium protein comprising SEQ ID NO: 72 (B. anthracis Sterne BAS1141 (ExsY)), an exosporium protein comprising SEQ ID NO: 73 (B. anthracis Sterne BAS1144 (BxpB/ExsFA)), an exosporium protein comprising SEQ ID NO: 74 (B. anthracis Sterne BAS1145 (CotY)), an exosporium protein comprising SEQ ID NO: 75 (B. anthracis Sterne BAS1140), an exosporium protein comprising SEQ ID NO: 76 (B. anthracis H9401 ExsFB), an exosporium protein comprising SEQ ID NO: 77 (B. thuringiensis HD74 InhA1), an exosporium protein comprising SEQ ID NO: 78 (B. cereus ATCC 10876 ExsJ), an exosporium protein comprising SEQ ID NO: 79 (B. cereus ExsH), an exosporium protein comprising SEQ ID NO: 80 (B. anthracis Ames YjcA), an exosporium protein comprising SEQ ID NO: 81 (B. anthracis YjcB), an exosporium protein comprising SEQ ID NO: 82 (B. anthracis Sterne BclC), an exosporium protein comprising SEQ ID NO: 83 (Bacillus thuringiensis serovar konkukian str. 97-27 acid phosphatase), or an exosporium protein comprising SEQ ID NO: 84 (B. thuringiensis HD74 InhA2). Inclusion of an exosporium protein comprising SEQ ID NO: 71, 72, 73, 74, 75, 76, 77, 78, 79, 80, 81, 82, 83, or 84 in the fusion proteins described herein will result in targeting to the exosporium of a B. cereus family member.

Moreover, exosporium proteins having a high degree of sequence identity with any of the full-length exosporium proteins or the exosporium protein fragments described above can also be used to target a peptide or protein to the exosporium of a Bacillus cereus family member. Thus, the fusion protein can comprise an exosporium protein comprising an amino acid sequence having at least 85% identity with any one of SEQ ID NOS: 2, 4, 6, 8, 10, 12, 14, 16, 18, 20, 22, 24, 26, 28, 30, 32, 34, 36, 44, 46, 48, 50, 52, 54, 56, 58, 59, 71, 72, 73, 74, 75, 76, 77, 78, 79, 80, 81, 82, 83, and 84. Alternatively, the fusion protein can comprise an exosporium protein having at least 90%, at least 95%, at least 98%, at least 99%, or 100% identity with any one of SEQ ID NOs: 2, 4, 6, 8, 10, 12, 14, 16, 18, 20, 22, 24, 26, 28, 30, 32, 34, 36, 44, 46, 48, 50, 52, 54, 56, 58, 59, 71, 72, 73, 74, 75, 76, 77, 78, 79, 80, 81, 82, 83, and 84.

Alternatively, the fusion protein can comprise an exosporium protein fragment consisting of an amino acid sequence having at least 85% identity with SEQ ID NO: 59. Alternatively, the fusion protein can comprise an exosporium protein fragment consisting of an amino acid sequence having at least 90%, at least 95%, at least 98%, at least 99%, or 100% identity with SEQ ID NO: 59.

In any of the targeting sequences, exosporium proteins, or exosporium protein fragments described herein, the targeting sequence, exosporium protein, or exosporium protein fragment can comprise the amino acid sequence GXT at its carboxy terminus, wherein X is any amino acid.

In any of the targeting sequences, exosporium proteins, and exosporium protein fragments described herein, the targeting sequence, exosporium protein, or exosporium protein fragment, can comprise an alanine residue at the position of the targeting sequence that corresponds to amino acid 20 of SEQ ID NO: 1.

### Fusion Proteins

The fusion proteins can comprise a targeting sequence, an exosporium protein, or an exosporium protein fragment, and at least one plant growth stimulating protein or peptide. The plant growth stimulating protein or peptide can comprise a peptide hormone, a non-hormone peptide, an enzyme involved in the production or activation of a plant growth stimulating compound or an enzyme that degrades or modifies a bacterial, fungal, or plant nutrient source. The targeting sequence, exosporium protein, or exosporium protein fragment can be any of the targeting sequences, exosporium proteins, or exosporium protein fragments described above.

The fusion proteins can comprise a targeting sequence, an exosporium protein, or an exosporium protein fragment, and at least one protein or peptide that protects a plant from a pathogen. The targeting sequence, exosporium protein, or exosporium protein fragment can be any of the targeting sequences, exosporium proteins, or exosporium protein fragments described above.

The fusion protein can be made using standard cloning and molecular biology methods known in the art. For example, a gene encoding a protein or peptide (e.g., a gene encoding a plant growth stimulating protein or peptide) can be amplified by polymerase chain reaction (PCR) and ligated to DNA coding for any of the above-described targeting sequences to form a DNA molecule that encodes the fusion protein. The DNA molecule encoding the fusion protein can be cloned into any suitable vector, for example a plasmid vector. The vector suitably comprises a multiple cloning site into which the DNA molecule encoding the fusion protein can be easily inserted. The vector also suitably contains a selectable marker, such as an antibiotic resistance gene, such that bacteria transformed, transfected, or mated with the vector can be readily identified and isolated. Where the vector is a plasmid, the plasmid suitably also comprises an origin of replication. The DNA encoding the fusion protein is suitably under the control of a sporulation promoter which will cause expression of the fusion protein on the exosporium of a B. cereus family member endospore (e.g., a native bclA promoter from a B. cereus family member). Alternatively, DNA coding for the fusion protein can be integrated into the chromosomal DNA of the B. cereus family member host.

The fusion protein can also comprise additional polypeptide sequences that are not part of the targeting sequence, exosporium protein, exosporium protein fragment, or the plant growth stimulating protein or peptide, the protein or peptide that protects a plant from a pathogen, the protein or peptide that enhances stress resistance in a plant, or the plant binding protein or peptide. For example, the fusion protein can include tags or markers to facilitate purification or visualization of the fusion protein (e.g., a polyhistidine tag or a fluorescent protein such as GFP or YFP) or visualization of recombinant exosporium-producing Bacillus cells spores expressing the fusion protein.

Expression of fusion proteins on the exosporium using the targeting sequences, exosporium proteins, and exosporium protein fragments described herein is enhanced due to a lack of secondary structure in the amino-termini of these sequences, which allows for native folding of the fused proteins and retention of activity. Proper folding can be further enhanced by the inclusion of a short amino acid linker between the targeting sequence, exosporium protein, exosporium protein fragment, and the fusion partner protein.

Thus, any of the fusion proteins described herein can comprise an amino acid linker between the targeting sequence, the exosporium protein, or the exosporium protein fragment and the plant growth stimulating protein or peptide, the protein or peptide that protects a plant from a pathogen, the protein or peptide that enhances stress resistance in a plant, or the plant binding protein or peptide.

The linker can comprise a polyalanine linker or a polyglycine linker. A linker comprising a mixture of both alanine and glycine residues can also be used. For example, where the targeting sequence comprises SEQ ID NO: 1, a fusion protein can have one of the following structures:
No linker: SEQ ID NO: 1-Fusion Partner Protein
Alanine Linker: SEQ ID NO: 1-An-Fusion Partner Protein
Glycine Linker: SEQ ID NO: 1-Gn-Fusion Partner Protein
Mixed Alanine and Glycine Linker: SEQ ID NO: 1-(A/G)n-Fusion Partner Protein
where An, Gn, and (A/G)n are any number of alanines, any number of glycines, or any number of a mixture of alanines and glycines, respectively. For example, n can be 1 to 25, and is preferably 6 to 10. Where the linker comprises a mixture of alanine and glycine residues, any combination of glycine and alanine residues can be used. In the above structures, “Fusion Partner Protein” represents the plant growth stimulating protein or peptide, the protein or peptide that protects a plant from a pathogen, the protein or peptide that enhances stress resistance in a plant, or the plant binding protein or peptide.

Alternatively or in addition, the linker can comprise a protease recognition site. Inclusion of a protease recognition site allows for targeted removal, upon exposure to a protease that recognizes the protease recognition site, of the plant growth stimulating protein or peptide, the protein or peptide that protects a plant from a pathogen, the protein or peptide that enhances stress resistance in a plant, or the plant binding protein or peptide.

### Plant Growth Stimulating Proteins and Peptides

As noted above, the fusion proteins can comprise a targeting sequence, exosporium protein, or exosporium protein fragment and at least one plant growth stimulating protein or peptide. For example, the plant growth stimulating protein or peptide can comprise a peptide hormone, a non-hormone peptide, an enzyme involved in the production or activation of a plant growth stimulating compound, or an enzyme that degrades or modifies a bacterial, fungal, or plant nutrient source.

For example, where the plant growth stimulating protein or peptide comprises a peptide hormone, the peptide hormone can comprise a phytosulfokine (e.g., phytosulfokine-α), clavata 3 (CLV3), systemin, ZmlGF, or a SCR/SP11.

Where the plant growth stimulating protein or peptide comprises a non-hormone peptide, the non-hormone peptide can comprise a RKN 16D10, Hg-Syv46, an eNOD40 peptide, melittin, mastoparan, Mas7, RHPP, POLARIS, or kunitz trypsin inhibitor (KTI).

The plant growth stimulating protein or peptide can comprise an enzyme involved in the production or activation of a plant growth stimulating compound. The enzyme involved in the production or activation of a plant growth stimulating compound can be any enzyme that catalyzes any step in a biological synthesis pathway for a compound that stimulates plant growth or alters plant structure, or any enzyme that catalyzes the conversion of an inactive or less active derivative of a compound that stimulates plant growth or alters plant structure into an active or more active form of the compound.

The plant growth stimulating compound can comprise a compound produced by bacteria or fungi in the rhizosphere, e.g., 2,3-butanediol.

Alternatively, the plant growth stimulating compound can comprise a plant growth hormone, e.g., a cytokinin or a cytokinin derivative, ethylene, an auxin or an auxin derivative, a gibberellic acid or a gibberellic acid derivative, abscisic acid or an abscisic acid derivative, or a jasmonic acid or a jasmonic acid derivative.

Where the plant growth stimulating compound comprises a cytokinin or a cytokinin derivative, the cytokinin or the cytokinin derivative can comprise kinetin, cis-zeatin, trans-zeatin, 6-benzylaminopurine, dihydroxyzeatin, N6-(D2-isopentenyl) adenine, ribosylzeatin, N6-(D2-isopentenyl) adenosine, 2-methylthio-cis-ribosylzeatin, cis-ribosylzeatin, trans-ribosylzeatin, 2-methylthio-trans-ribosylzeatin, ribosylzeatin-5-monosphosphate, N6-methylaminopurine, N6-dimethylaminopurine, 2′-deoxyzeatin riboside, 4-hydroxy-3-methyl-trans-2-butenylaminopurine, ortho-topolin, meta-topolin, benzyladenine, ortho-methyltopolin, meta-methyltopolin, or a combination thereof.

Where the plant growth stimulating compound comprises an auxin or an auxin derivative, the auxin or the auxin derivative can comprise an active auxin, an inactive auxin, a conjugated auxin, a naturally occurring auxin, or a synthetic auxin, or a combination thereof. For example, the auxin or auxin derivative can comprise indole-3-acetic acid, indole-3-pyruvic acid, indole-3-acetaldoxime, indole-3-acetamide, indole-3-acetonitrile, indole-3-ethanol, indole-3-pyruvate, indole-3-acetaldoxime, indole-3-butyric acid, a phenylacetic acid, 4-chloroindole-3-acetic acid, a glucose-conjugated auxin, or a combination thereof.

The enzyme involved in the production or activation of a plant growth stimulating compound can comprise an acetoin reductase, an indole-3-acetamide hydrolase, a tryptophan monooxygenase, an acetolactate synthetase, an α-acetolactate decarboxylase, a pyruvate decarboxylase, a diacetyl reductase, a butanediol dehydrogenase, an aminotransferase (e.g., tryptophan aminotransferase), a tryptophan decarboxylase, an amine oxidase, an indole-3-pyruvate decarboxylase, an indole-3-acetaldehyde dehydrogenase, a tryptophan side chain oxidase, a nitrile hydrolase, a nitrilase, a peptidase, a protease, an adenosine phosphate isopentenyltransferase, a adenosine kinase, an adenine phosphatase, an phosphoribosyltransferase, CYP735A, a 5′ribonucleotide phosphohydrolase, an adenosine nucleosidase, a zeatin cis-trans isomerase, a zeatin O-glucosyltransferase, a β-glucosidase, a cis-hydroxylase, a CK cis-hydroxylase, a CK N-glucosyltransferase, a 2,5-ribonucleotide phosphohydrolase, an adenosine nucleosidase, a purine nucleoside phosphorylase, a zeatin reductase, a hydroxylamine reductase, a 2-oxoglutarate dioxygenase, a gibberellic 2B/3B hydrolase, a gibberellin 3-oxidase, a gibberellin 20-oxidase, a chitosinase, a chitinase, a β-1,3-glucanase, a β-1,4-glucanase, a β-1,6-glucanase, an aminocyclopropane-1-carboxylic acid deaminase, or an enzyme involved in producing a nod factor (e.g., nodA, nodB, or nodI).

Where the enzyme comprises a protease or peptidase, the protease or peptidase can be a protease or peptidase that cleaves proteins, peptides, proproteins, or preproproteins to create a bioactive peptide. The bioactive peptide can be any peptide that exerts a biological activity.

Examples of bioactive peptides include RKN 16D10 and RHPP.

The protease or peptidase that cleaves proteins, peptides, proproteins, or preproproteins to create a bioactive peptide can comprise subtilisin, an acid protease, an alkaline protease, a proteinase, an endopeptidase, an exopeptidase, thermolysin, papain, pepsin, trypsin, pronase, a carboxylase, a serine protease, a glutamic protease, an aspartate protease, a cysteine protease, a threonine protease, or a metalloprotease.

The protease or peptidase can cleave proteins in a protein-rich meal (e.g., soybean meal or yeast extract).

The plant growth stimulating protein can also comprise an enzyme that degrades or modifies a bacterial, fungal, or plant nutrient source. Such enzymes include cellulases, lipases, lignin oxidases, proteases, glycoside hydrolases, phosphatases, nitrogenases, nucleases, amidases, nitrate reductases, nitrite reductases, amylases, ammonia oxidases, ligninases, glucosidases, phospholipases, phytases, pectinases, glucanases, sulfatases, ureases, xylanases, and siderophores. When introduced into a plant growth medium or applied to a plant, seed, or an area surrounding a plant or a plant seed, fusion proteins comprising enzymes that degrade or modify a bacterial, fungal, or plant nutrient source can aid in the processing of nutrients in the vicinity of the plant and result in enhanced uptake of nutrients by the plant or by beneficial bacteria or fungi in the vicinity of the plant.

Suitable cellulases include endocellulases (e.g., an endogluconase such as a Bacillus subtilis endoglucanase, a Bacillus thuringiensis endoglucanase, a Bacillus cereus endoglucanase, or a Bacillus clausii endoglucanase), exocellulases (e.g., a Trichoderma reesei exocellulase), and β-glucosidases (e.g., a Bacillus subtilis β-glucosidase, a Bacillus thuringiensis β-glucosidase, a Bacillus cereus β-glucosidase, or a Bacillus clausii β-glucosidase).

The lipase can comprise a Bacillus subtilis lipase, a Bacillus thuringiensis lipase, a Bacillus cereus lipase, or a Bacillus clausii lipase.

In one embodiment, the lipase comprises a Bacillus subtilis lipase. The Bacillus subtilis lipase can be PCR amplified using the following primers:
ggatccatggctgaacacaatcc (forward, SEQ ID NO: 37) and ggatccttaattegtattctggcc (reverse, SEQ ID NO: 38).

In another embodiment, the cellulase is a Bacillus subtilis endoglucanase. The Bacillus subtilis endoglucanase can be PCR amplified using the following primers: ggatccatgaaacggtcaatc (forward, SEQ ID NO: 39) and ggatccttactaatttggttctgt (reverse, SEQ ID NO: 40).

In yet another embodiment, the fusion protein comprises an E. coli protease PtrB. The E. coli protease PtrB can be PCR amplified using the following primers: ggatccatgctaccaaaagcc (forward, SEQ ID NO: 41) and ggatccttagtccgcaggegtage (reverse, SEQ ID NO: 42).

In certain embodiments, the fusion protein contains an endoglucanase which derives from the nucleotide sequence in SEQ ID NO: 104.

The amino acid sequence for an exemplary endoglucanase that may be fused to the targeting sequence, an exosporium protein, or an exosporium protein fragment and, optionally, a linker sequence, such as a poly-A linker, is the fusion protein provided as SEQ ID NO: 107.

In other embodiments, the fusion protein contains a phospholipase that derives from the nucleotide sequence set forth in SEQ ID NO: 105.

The amino acid sequence for an exemplary phospholipase that may be fused to the targeting sequence, an exosporium protein, or an exosporium protein fragment and, optionally, a linker sequence, such as a poly-A linker, is the fusion protein provided as SEQ ID NO: 108.

In still other embodiments, the fusion protein contains a chitosanase that derives from the nucleotide sequence set forth in SEQ ID NO: 106. The amino acid sequence for an exemplary chitosanase that may be fused to the targeting sequence, an exosporium protein, or an exosporium protein fragment and, optionally, a linker sequence, such as a poly-A linker, in the fusion protein is provided as SEQ ID NO: 109.

To create fusion constructs, genes may be fused to the native bclA promoter of Bacillus thuringiensis DNA encoding the first 35 amino acids of BclA (amino acids 1-35 of SEQ ID NO: 1) using the splicing by overlapping extension (SOE) technique. Correct amplicons are cloned into the E. coli/Bacillus shuttle vector pHP13, and correct clones screened by DNA sequencing. Correct clones are electroporated into Bacillus thuringiensis (Cry-, plasmid-) and screened for chloramphenicol resistance. Correct transformants are grown in brain heart infusion broth overnight at 30° C., plated onto nutrient agar plates, and incubated at 30° C. for 3 days. Spores expressing the fusion construct (BEMD spores) may be collected off of the plates by washing in phosphate buffered saline (PBS) and purified by centrifugation and additional washes in PBS.

In such fusion proteins, the endoglucanase, phospholipase or chitosinase can comprise a nucleotide sequence encoding an amino acid sequence having at least 85% identity with SEQ ID NO: 107, 108 or 109, respectively.

In such fusion proteins, the endoglucanase, phospholipase or chitosinase can comprise an amino acid sequence having at least 90% identity with SEQ ID NO: 107, 108 or 109, respectively.

In such fusion proteins, the endoglucanase, phospholipase or chitosinase can comprise an amino acid sequence having at least 95% identity with SEQ ID NO: 107, 108 or 109, respectively.

In such fusion proteins, the endoglucanase, phospholipase or chitosinase can comprise an amino acid sequence having at least 98% identity with SEQ ID NO: 107, 108 or 109, respectively.

In such fusion proteins, the endoglucanase, phospholipase or chitosinase can comprise an amino acid sequence having at least 99% identity with SEQ ID NO: 107, 108 or 109, respectively.

Suitable lignin oxidases comprise lignin peroxidases, laccases, glyoxal oxidases, ligninases, and manganese peroxidases.

The protease can comprise a subtilisin, an acid protease, an alkaline protease, a proteinase, a peptidase, an endopeptidase, an exopeptidase, a thermolysin, a papain, a pepsin, a trypsin, a pronase, a carboxylase, a serine protease, a glutamic protease, an aspartate protease, a cysteine protease, a threonine protease, or a metalloprotease.

The phosphatase can comprise a phosphoric monoester hydrolase, a phosphomonoesterase (e.g., PhoA4), a phosphoric diester hydrolase, a phosphodiesterase, a triphosphoric monoester hydrolase, a phosphoryl anhydride hydrolase, a pyrophosphatase, a phytase (e.g., Bacillus subtilis EE148 phytase or Bacillus thuringiensis BT013A phytase), a trimetaphosphatase, or a triphosphatase.

The nitrogenase can comprise a Nif family nitrogenase (e.g., Paenibacillus massiliensis NifBDEHKNXV).

Proteins and Peptides that Protects Plants from Pathogens

The fusion proteins can comprise a targeting sequence, exosporium protein, or exosporium protein fragment, and at least one protein or peptide that protects a plant from a pathogen.

The protein or peptide can comprise a protein or peptide that stimulates a plant immune response. For example, the protein or peptide that stimulates a plant immune response can comprise a plant immune system enhancer protein or peptide. The plant immune system enhancer protein or peptide can be any protein or peptide that has a beneficial effect on the immune system of a plant. Suitable plant immune system enhancer proteins and peptides include harpins, α-elastins, β-elastins, systemins, phenylalanine ammonia-lyase, clicitins, defensins, cryptogeins, flagellin proteins, and flagellin peptides (e.g., flg22).

Alternatively, the protein or peptide that protects a plant from a pathogen can be a protein or peptide that has antibacterial activity, antifungal activity, or both antibacterial and antifungal activity. Examples of such proteins and peptides include bacteriocins, lysozymes, lysozyme peptides (e.g., LysM), siderophores, non-ribosomal active peptides, conalbumins, albumins, lactoferrins, lactoferrin peptides (e.g., LfcinB), streptavidin, and TasA.

The protein or peptide that protects a plant from a pathogen can also be a protein or peptide that has insecticidal activity, helminthicidal activity, suppresses insect or worm predation, or a combination thereof. For example, the protein or peptide that protects a plant from a pathogen can comprise an insecticidal bacterial toxin (e.g., a VIP insecticidal protein), an endotoxin, a Cry toxin (e.g., a Cry toxin from Bacillus thuringiensis), a protease inhibitor protein or peptide (e.g., a trypsin inhibitor or an arrowhead protease inhibitor), a cysteine protease, or a chitinase. Where the Cry toxin is a Cry toxin from Bacillus thuringiensis, the Cry toxin can be a Cry5B protein or a Cry21A protein. Cry5B and Cry21A have both insecticidal and nematocidal activity.

The protein that protects a plant from a pathogen can comprise an enzyme. Suitable enzymes include proteases and lactonases. The proteases and lactonases can be specific for a bacterial signaling molecule (e.g., a bacterial lactone homoserine signaling molecule).

Where the enzyme is a lactonase, the lactonase can comprise 1,4-lactonase, 2-pyrone-4,6-dicarboxylate lactonase, 3-oxoadipate enol-lactonase, actinomycin lactonase, deoxylimonate A-ring-lactonase, gluconolactonase L-rhamnono-1,4-lactonase, limonin-D-ring-lactonase, steroid-lactonase, triacetate-lactonase, or xylono-1,4-lactonase.

The enzyme can also be an enzyme that is specific for a cellular component of a bacterium or fungus. For example, the enzyme can comprise a β-1,3-glucanase, a β-1,4-glucanase, a β-1,6-glucanase, a chitosinase, a chitinase, a chitosinase-like enzyme, a lyticase, a peptidase, a proteinase, a protease (e.g., an alkaline protease, an acid protease, or a neutral protease), a mutanolysin, a stapholysin, or a lysozyme.

Proteins and Peptides that Enhance Stress Resistance in Plants

The fusion proteins can comprise a targeting sequence, exosporium protein, or exosporium protein fragment and at least one protein or peptide that enhances stress resistance in a plant.

For example, the protein or peptide that enhances stress resistance in a plant comprises an enzyme that degrades a stress-related compound. Stress-related compounds include, but are not limited to, aminocyclopropane-1-carboxylic acid (ACC), reactive oxygen species, nitric oxide, oxylipins, and phenolics. Specific reactive oxygen species include hydroxyl, hydrogen peroxide, oxygen, and superoxide. The enzyme that degrades a stress-related compound can comprise a superoxide dismutase, an oxidase, a catalase, an aminocyclopropane-1-carboxylic acid deaminase, a peroxidase, an antioxidant enzyme, or an antioxidant peptide.

The protein or peptide that enhances stress resistance in a plant can also comprise a protein or peptide that protects a plant from an environmental stress. The environmental stress can comprise, for example, drought, flood, heat, freezing, salt, heavy metals, low pH, high pH, or a combination thereof. For instance, the protein or peptide that protects a plant from an environmental stress can comprises an ice nucleation protein, a prolinase, a phenylalanine ammonia lyase, an isochorismate synthase, an isochorismate pyruvate lyase, or a choline dehydrogenase.

### Plant Binding Proteins and Peptides

The fusion proteins can comprise a targeting sequence, exosporium protein, or exosporium protein fragment and at least plant binding protein or peptide. The plant binding protein or peptide can be any protein or peptide that is capable of specifically or non-specifically binding to any part of a plant (e.g., a plant root or an aerial portion of a plant such as a leaf, stem, flower, or fruit) or to plant matter. Thus, for example, the plant binding protein or peptide can be a root binding protein or peptide, or a leaf binding protein or peptide.

Suitable plant binding proteins and peptides include adhesins (e.g., rhicadhesin), flagellins, omptins, lectins, expansins, biofilm structural proteins (e.g., TasA or YuaB) pilus proteins, curlus proteins, intimins, invasins, agglutinins, and afimbrial proteins.

Recombinant Bacillus that Express the Fusion Proteins

The fusion proteins described herein can be expressed by recombinant exosporium-producing Bacillus cells. The fusion protein can be any of the fusion proteins discussed above.

The recombinant exosporium-producing Bacillus cells can coexpress two or more of any of the fusion proteins discussed above. For example, the recombinant exosporium-producing Bacillus cells can coexpress at least one fusion protein that comprises a plant binding protein or peptide, together with at least one fusion protein comprising a plant growth stimulating protein or peptide, at least one fusion protein comprising a protein or peptide that protects a plant from a pathogen, or at least one protein or peptide that enhances stress resistance in a plant.

The recombinant exosporium-producing Bacillus cells can comprise Bacillus anthracis, Bacillus cereus, Bacillus thuringiensis, Bacillus mycoides, Bacillus pseudomycoides, Bacillus samanii, Bacillus gaemokensis, Bacillus weihenstephensis, Bacillus toyoiensis or a combination thereof. For example, the recombinant exosporium-producing Bacillus cells can comprise Bacillus cereus, Bacillus thuringiensis, Bacillus pseudomycoides, or Bacillus mycoides. In particular, the recombinant exosporium-producing Bacillus cells can comprise Bacillus thuringiensis or Bacillus mycoides.

To generate a recombinant exosporium-producing Bacillus cells expressing a fusion protein, any Bacillus cereus family member can be conjugated, transduced, or transformed with a vector encoding the fusion protein using standard methods known in the art (e.g., by electroporation). The bacteria can then be screened to identify transformants by any method known in the art. For example, where the vector includes an antibiotic resistance gene, the bacteria can be screened for antibiotic resistance. Alternatively, DNA encoding the fusion protein can be integrated into the chromosomal DNA of a B. cereus family member host. The recombinant exosporium-producing Bacillus cells can then exposed to conditions which will induce sporulation. Suitable conditions for inducing sporulation are known in the art. For example, the recombinant exosporium-producing Bacillus cells can be plated onto agar plates, and incubated at a temperature of about 30° C. for several days (e.g., 3 days).

Inactivated strains, non-toxic strains, or genetically manipulated strains of any of the above species can also suitably be used. For example, a Bacillus thuringiensis that lacks the Cry toxin can be used. Alternatively or in addition, once the recombinant B. cereus family spores expressing the fusion protein have been generated, they can be inactivated to prevent further germination once in use. Any method for inactivating bacterial spores that is known in the art can be used. Suitable methods include, without limitation, heat treatment, gamma irradiation, x-ray irradiation, UV-A irradiation, UV-B irradiation, chemical treatment (e.g., treatment with gluteraldehyde, formaldehyde, hydrogen peroxide, acetic acid, bleach, or any combination thereof), or a combination thereof. Alternatively, spores derived from nontoxigenic strains, or genetically or physically inactivated strains, can be used.

Recombinant Exosporium-Producing Bacillus Cells Having Plant-Growth Promoting Effects and/or Other Beneficial Attributes

Many Bacillus cereus family member strains have inherent beneficial attributes. For example, some strains have plant-growth promoting effects. Any of the fusion proteins described herein can be expressed in such strains.

For example, the recombinant exosporium-producing Bacillus cells can comprise a plant-growth promoting strain of bacteria.

The plant-growth promoting strain of bacteria can comprise a strain of bacteria that produces an insecticidal toxin (e.g., a Cry toxin), produces a fungicidal compound (e.g., a β-1,3-glucanase, a chitosinase, a lyticase, or a combination thereof), produces a nematocidal compound (e.g., a Cry toxin), produces a bactericidal compound, is resistant to one or more antibiotics, comprises one or more freely replicating plasmids, binds to plant roots, colonizes plant roots, forms biofilms, solubilizes nutrients, secretes organic acids, or any combination thereof.

For example, where the recombinant exosporium-producing Bacillus cells comprises a plant-growth promoting strain of bacteria, the plant growth-promoting strain of bacteria can comprise Bacillus mycoides BT155 (NRRL No. B-50921), Bacillus mycoides EE118 (NRRL No. B-50918), Bacillus mycoides EE141 (NRRL No. B-50916), Bacillus mycoides BT46-3 (NRRL No. B-50922), Bacillus cereus family member EE128 (NRRL No. B-50917), Bacillus thuringiensis BT013A (NRRL No. B-50924), or Bacillus cereus family member EE349 (NRRL No. B-50928). Bacillus thuringiensis BT013A is also known as Bacillus thuringiensis 4Q7. Each of these strains was deposited with the United States Department of Agriculture (USDA) Agricultural Research Service (ARS), having the address 1815 North University Street, Peoria, Illinois 61604, U.S.A., on Mar. 10, 2014, and is identified by the NRRL deposit number provided in parentheses.

These plant-growth promoting strains were isolated from the rhizospheres of various vigorous plants and were identified by their 16S rRNA sequences, and through biochemical assays. The strains were identified at least to their genus designation by means of conventional biochemistry and morphological indicators. Biochemical assays for confirmed Gram-positive strains such as Bacillus included growth on PEA medium and nutrient agar, microscopic examination, growth on 5% and 7.5% NaCl medium, growth at pH 5 and pH 9, growth at 42° C. and 50° C., the ability to produce acid upon fermentation with cellobiose, lactose, glycerol, glucose, sucrose, d-mannitol, and starch; fluorescent pigment production; gelatin hydrolysis; nitrate reduction; catalase production, starch hydrolysis; oxidase reaction, urease production and motility.

For example, the recombinant exosporium-producing Bacillus cells comprising a plant-growth promoting strain of bacteria can comprise Bacillus mycoides BT155, Bacillus mycoides EE141, or Bacillus thuringiensis BT013A. The recombinant exosporium-producing Bacillus cells can express any of the fusion proteins described herein, e.g., a fusion protein comprising the targeting sequence of SEQ ID NO: 60 and a non-hormone peptide (e.g., kunitz trypsin inhibitor (KTI), an enzyme involved in the production or activation of a plant growth stimulating compound (e.g., a chitosinase), a plant binding protein or peptide (e.g., TasA); a protein or peptide that protects a plant from a pathogen (e.g., TasA), or an enzyme that degrades or modifies a bacterial, fungal, or plant nutrient source (e.g., a phosphatase such as PhoA or phytase, or an endoglucanase).

### Promoters

In any of the recombinant exosporium-producing Bacillus cells described herein, the fusion protein can be expressed under the control of a promoter that is native to the targeting sequence, the exosporium protein, or the exosporium protein fragment of the fusion protein. For example, where the fusion protein comprises a targeting sequence derived from B. anthracis Sterne BclA (e.g., amino acids 20-35 of SEQ ID NO: 1, amino acids 1-35 of SEQ ID NO: 1, SEQ ID NO: 1, or SEQ ID NO: 60) or where the fusion protein comprises full length BclA (SEQ ID NO: 2) or a fragment of full length BclA (e.g., SEQ ID NO: 59), the fusion protein can be expressed under the control of a promoter that is normally associated with the BclA gene in the genome of B. anthracis Sterne (e.g., the promoter of SEQ ID NO: 85).

Alternatively, the fusion protein can be expressed under the control of a high-expression sporulation promoter. In some cases, the promoter that is native to the targeting sequence, exosporium protein, or exosporium protein fragment will be a high-expression sporulation promoter. In other cases, the promoter that is native to the targeting sequence, exosporium protein, or exosporium protein fragment will not be a high-expression sporulation promoter. In the latter cases, it may be advantageous to replace the native promoter with a high-expression sporulation promoter. Expression of the fusion protein under the control of a high-expression sporulation promoter provides for increased expression of the fusion protein on the exosporium of the Bacillus cereus family member.

The high-expression sporulation promoter can comprise one or more sigma-K sporulation-specific polymerase promoter sequences.

Suitable high-expression sporulation promoters for use in expressing the fusion proteins in a Bacillus cereus family member include those listed in Table 2 below:

TABLE 2
Promoter Sequences

| Promoter | |
|---|---|
| (SEQ ID NO.) | Sequence |
| BclA promoter | TAATCACCCTCTTCCAAATCAATCATATGTTATACATATACTAAACT |
| (B. anthracis Sterne) | TTCCATTTTTTTAAATTGTTCAAGTAGTTTAAGATTTCTTTTCAATAAT |
| (SEQ ID NO: 85) | TCAAATGTCCGTGTCATTTTCTTTCGGTTTTGCATCTACTATATAATG |
| | AACGCTTTATGGAGGTGAATTTATG |
| BetA promoter | ATTTATTTCATTCAATTTTTCCTATTTAGTACCTACCGCACTCACAAAA |
| (B. anthracis Sterne) | AGCACCTCTCATTAATTTATATTATAGTCATTGAAATCTAATTTAATGA |
| (SEQ ID NO: 86) | AATCATCATACTATATGTTTTATAAGAAGTAAAGGTACCATACTTAA |
| | TTAATACATATCTATACACTTCAATATCACAGCATGCAGTTGAATTAT |
| | ATCCAACTTTCATTTCAAATTAAATAAGTGCCTCCGCTATTGTGAATG |
| | TCATTTACTCTCCCTACTACATTTAATAATTATGACAAGCAATCATAG |
| | GAGGTTACTACATG |
| BAS1882 promoter | AATTACATAACAAGAACTACATTAGGGAGCAAGCAGTCTAGCGAAAG |
| (B. anthracis Sterne) | CTAACTGCTTTTTTATTAAATAACTATTTTATTAAATTTCATATATACA |
| (SEQ ID NO: 87) | ATCGCTTGTCCATTTCATTTGGCTCTACCCACGCATTTACTATTAGTA |
| | ATATGAATTTTTCAGAGGTGGATTTTATT |
| Gene 3572 promoter | CTATGATTTAAGATACACAATAGCAAAAGAGAAACATATTATATAAC |
| (B. weihenstephensis | GATAAATGAAACTTATGTATATGTATGGTAACTGTATATATTACTACA |
| KBAB 4) | ATACAGTATACTCATAGGAGGTAGGTATG |
| (SEQ ID NO: 88) | |
| YVTN β-propeller | GGTAGGTAGATTTGAAATATGATGAAGAAAAGGAATAACTAAAAGGA |
| protein promoter | GTCGATATCCGACTCCTTTTAGTTATAAATAATGTGGAATTAGAGTAT |
| (B. weihenstephensis | AATTTTATATAGGTATATTGTATTAGATGAACGCTTTATCCTTTAATTG |
| KBAB 4) | TGATTAATGATGGATTGTAAGAGAAGGGGCTTACAGTCCTTTTTTTAT |
| (SEQ ID NO: 89) | GGTGTTCTATAAGCCTTTTTAAAAGGGGTACCACCCCACACCCAAAAA |
| | CAGGGGGGGTTATAACTACATATTGGATGTTTTGTAACGTACAAGAAT |
| | CGGTATTAATTACCCTGTAAATAAGTTATGTGTATATAAGGTAACTTT |
| | ATATATTCTCCTACAATAAAATAAAGGAGGTAATAAAGTG |
| Cry1A promoter | AACCCTTAATGCATTGGTTAAACATTGTAAAGTCTAAAGCATGGATAA |
| (B. thuringiensis | TGGGCGAGAAGTAAGTAGATTGTTAACACCCTGGGTCAAAAATTGAT |
| HD-73) | ATTTAGTAAAATTAGTTGCACTTTGTGCATTTTTTCATAAGATGAGTC |
| (SEQ ID NO: 90) | ATATGTTTTAAATTGTAGTAATGAAAAACAGTATTATATCATAATGA |
| | ATTGGTATCTTAATAAAAGAGATGGAGGTAACTTA |
| ExsY promoter | TAATTCCACCTTCCCTTATCCTCTTTCGCCTATTTAAAAAAAGGTCTTG |
| (B. thuringiensis | AGATTGTGACCAAATCTCCTCAACTCCAATATCTTATTAATGTAAATA |
| serovar konkukian str. | CAAACAAGAAGATAAGGAGTGACATTAA |
| 97-27) | |
| (SEQ ID NO: 91) | |
| CotY promoter | AGGATGTCTTTTTTTATATTGTATTATGTACATCCCTACTATATAAATT |
| (B. thuringiensis Al | CCCTGCTTTTATCGTAAGAATTAACGTAATATCAACCATATCCCGTTC |
| Hakam) | ATATTGTAGTAGTGTATGTCAGAACTCACGAGAAGGAGTGAACATAA |
| (SEQ ID NO: 92) | |
| YjcA promoter | TTAATGTCACTCCTTATCTTCTTGTTTGTATTTACATTAATAAGATATT |
| (B. thuringiensis | GGAGTTGAGGAGATTTGGTCACAATCTCAAGACCTTTTTTTTAAATAG |
| serovar kurstaki str. | GCGAAAGAGGATAAGGGAAGGTGGAATTA |
| HD73) | |
| (SEQ ID NO: 93) | |
| YjcB promoter | ATATATTTTCATAATACGAGAAAAAGCGGAGTTTAAAAGAATGAGGG |
| (B. thuringiensis | AACGGAAATAAAGAGTTGTTCATATAGTAAATAGACAGAATTGACAG |
| serovar kurstaki str. | TAGAGGAGA |
| HD73) | |
| (SEQ ID NO: 94) | |
| BxpB promoter | AAACTAAATAATGAGCTAAGCATGGATTGGGTGGCAGAATTATCTGC |
| (B. thuringiensis Al | CACCCAATCCATGCTTAACGAGTATTATTATGTAAATTTCTTAAAATT |
| Hakam) | GGGAACTTGTCTAGAACATAGAACCTGTCCTTTTCATTAACTGAAAG |
| (SEQ ID NO: 95) | TAGAAACAGATAAAGGAGTGAAAAACA |
| Rhamnose promoter | ATTCACTACAACGGGGATGAGTTTGATGCGGATACATATGAGAAGTA |
| (B. thuringiensis Al | CCGGAAAGTGTTTGTAGAACATTACAAAGATATATTATCTCCATCATA |
| Hakam) | AAGGAGAGATGCAAAG |
| (SEQ ID NO: 96) | |
| CotY/CotZ promoter | CGCGCACCACTTCGTCGTACAACAACGCAAGAAGAAGTTGGGGATAC |
| (B. anthracis Sterne) | AGCAGTATTCTTATTCAGTGATTTAGCACGCGGCGTAACAGGAGAAA |
| (SEQ ID NO: 97) | ACATTCACGTTGATTCAGGGTATCATATCTTAGGATAAATATAATATT |
| | AATTTTAAAGGACAATCTCTACATGTTGAGATTGTCCTTTTTATTTGTT |
| | CTTAGAAAGAACGATTTTTAACGAAAGTTCTTACCACGTTATGAATAT |
| | AAGTATAATAGTACACGATTTATTCAGCTACGTA |
| BclC promoter | TGAAGTATCTAGAGCTAATTTACGCAAAGGAATCTCAGGACAACACT |
| (B. anthracis Sterne) | TTCGCAACACCTATATTTTAAATTTAATAAAAAAAGAGACTCCGGAGT |
| (SEQ ID NO: 98) | CAGAAATTATAAAGCTAGCTGGGTTCAAATCAAAAATTTCACTAAAA |
| | CGATATTATCAATACGCAGAAAATGGAAAAAACGCCTTATCATAAGG |
| | CGTTTTTTCCATTTTTTCTTCAAACAAACGATTTTACTATGACCATTTA |
| | ACTAATTTTTGCATCTACTATGATGAGTTTCATTCACATTCTCATTAG |
| | AAAGGAGAGATTTAATG |
| Sigma K promoter | TATATCATATGTAAAATTAGTTCTTATTCCCACATATCATATAGAATC |
| (B. anthracis Sterne) | GCCATATTATACATGCAGAAAACTAAGTATGGTATTATTCTTAAATTG |
| (SEQ ID NO: 99) | TTTAGCACCTTCTAATATTACAGATAGAATCCGTCATTTTCAACAGTG |
| | AACATGGATTTCTTCTGAACACAACTCTTTTTCTTTCCTTATTTCCAAA |
| | AAGAAAAGCAGCCCATTTTAAAATACGGCTGCTTGTAATGTACATTA |
| InhA promoter | TATCACATAACTCTTTATTTTTAATATTTCGACATAAAGTGAAACTTT |
| (B. thuringiensis Al | AATCAGTGGGGGCTTTGTTCATCCCCCCACTGATTATTAATTGAACCA |
| Hakam) | AGGGATAAAAAGATAGAGGGTCTGACCAGAAAACTGGAGGGCATGA |
| (SEQ ID NO: 100) | TTCTATAACAAAAAGCTTAATGTTTATAGAATTATGTCTTTTTATATAG |
| | GGAGGGTAGTAAACAGAGATTTGGACAAAAATGCACCGATTTATCTG |
| | AATTTTAAGTTTTATAAAGGGGAGAAATG |
| BclA cluster glycosyl | ATTTTTTACTTAGCAGTAAAACTGATATCAGTTTTACTGCTTTTTCATT |
| transferase operon 1 | TTTAAATTCAATCATTAAATCTTCCTTTTCTACATAGTCATAATGTTGT |
| (B. thuringiensis | ATGACATTCCGTAGGAGGCACTTATA |
| serovar konkukian str. | |
| 97-27) | |
| (SEQ ID NO: 101) | |
| BclA cluster glycosyl | ACATAAATTCACCTCCATAAAGCGTTCATTATATAGTAGATGCAAAAC |
| transferase operon 2 | CGAAAGAAAATGACACGGACATTTGAATTATTGAAAAGAAATCTTAA |
| (B. thuringiensis | ACTACTTGAACAATTTAAAAAAATGGAAAGTTTAGTATATGTATAAC |
| serovar kurstaki str. | ATATGATTGATTTGGAAGAGGGTGATTA |
| HD73) | |
| (SEQ ID NO: 102) | |
| Glycosyl transferase | TTCTATTTTCCAACATAACATGCTACGATTAAATGGTTTTTTGCAAAT |
| promoter | GCCTTCTTGGGAAGAAGGATTAGAGCGTTTTTTTATAGAAACCAAAAG |
| (B. thuringiensis Al | TCATTAACAATTTTAAGTTAATGACTTTTTTGTTTGCCTTTAAGAGGTT |
| Hakam) | TTATGTTACTATAATTATAGTATCAGGTACTAATAACAAGTATAAGTA |
| (SEQ ID NO: 103) | TTTCTGGGAGGATATATCA |

In the promoter sequences listed in Table 2 above, the locations of the sigma-K sporulation-specific polymerase promoter sequences are indicated by bold and underlined text. The Cry1A promoter (B. thuringiensis HD-73; SEQ ID NO: 90) has a total of four sigma-K sequences, two of which overlap with one another, as indicated by the double underlining in Table 2.

Preferred high-expression sporulation promoters for use in expressing the fusion proteins in a Bacillus cereus family member include the BetA promoter (B. anthracis Sterne; SEQ ID NO: 86), the BclA promoter (B. anthracis Sterne; SEQ ID NO: 85), the BclA cluster glycosyl transferase operons 1 and 2 promoters (B. anthracis Sterne; SEQ ID NOs: 101 and 102), and the YVTN B-propeller protein promoter (B. weihenstephensis KBAB 4; SEQ ID NO: 89).

In any of the recombinant exosporium-producing Bacillus cells described herein, the fusion protein can be expressed under the control of a sporulation promoter comprising a nucleic acid sequence having at least 80%, at least 90%, at least 95%, at least 98%, at least 99%, or 100% identity with a nucleic acid sequence of any one of SEQ ID NOS: 85-103.

When the sporulation promoter comprising a nucleic acid sequence having at least 80%, at least 90%, at least 95%, at least 98%, or at least 99% identity with a nucleic acid sequence of any one of SEQ ID NOS: 85-103, the sigma-K sporulation-specific polymerase promoter sequence or sequences preferably have 100% identity with the corresponding nucleotides of SEQ ID NO: 85, 86, 87, 88, 89, 90, 91, 92, 93, 94, 95, 96, 97, 98, 99, 100, 101, 102, or 103. For example, as illustrated in Table 2 above, the BclA promoter of B. anthracis Sterne (SEQ ID NO: 85) has sigma-K sporulation-specific polymerase promoter sequences at nucleotides 24-32, 35-43, and 129-137. Thus, if the sporulation promoter comprises a sequence having at least 90% identity with the nucleic acid sequence of SEQ ID NO: 85, it is preferred that the nucleotides of the sporulation promoter corresponding to nucleotides 24-32, 35-43, and 129-137 of SEQ ID NO: 85 have 100% identity with nucleotides 24-32, 35-43, and 129-137 of SEQ ID NO: 85.

In any of the methods described herein for stimulating plant growth, plants grown in the plant growth medium comprising the recombinant exosporium-producing Bacillus cells and at least one insecticide selected from the particular insecticides disclosed herein exhibit increased growth as compared to the growth of plants in the identical plant growth medium that does not contain the recombinant exosporium-producing Bacillus cells.

In any of the compositions and methods described herein for stimulating plant growth, the recombinant exosporium-producing Bacillus cells can comprise any of the recombinant plant-growth promoting strains of bacteria described above.

In any of the compositions or methods for stimulating plant growth disclosed herein, the fusion protein can be expressed under the control of any of the promoters described above.

### Insecticides

“Insecticides” as well as the term “insecticidal” refers to the ability of a substance to increase mortality or inhibit growth rate of insects. As used herein, the term “insects” includes all organisms in the class “Insecta”. The term “pre-adult” insects refers to any form of an organism prior to the adult stage, including, for example, eggs, larvae, and nymphs. As used herein, the terms “insecticide” and “insecticidal” also encompass “nematicide” and “nematicidal” and “acaricide” and “acaricidal.”

“Nematicides” and “nematicidal” refers to the ability of a substance to increase mortality or inhibit the growth rate of nematodes. In general, the term “nematode” comprises eggs, larvae, juvenile and mature forms of said organism.

“Acaricide” and “acaricidal” refers to the ability of a substance to increase mortality or inhibit growth rate of ectoparasites belonging to the class Arachnida, sub-class Acari.

The active ingredients specified herein by their “common name” are known and described, for example, in the pesticide handbook (“The Pesticide Manual,” 16th Ed., British Crop Protection Council 2012) or can be found on the Internet (e.g. http://www.alanwood.net/pesticides).

According to one embodiment of the present invention the at least one insecticide is selected from the group consisting of
(1) Acetylcholinesterase (AChE) inhibitors, such as, for example, carbamates, for example alanycarb, bendiocarb, benfuracarb, butocarboxim, butoxycarboxim, carbofuran, carbosulfan, ethiofencarb, furathiocarb, isoprocarb, metolcarb, oxamyl, pirimicarb, propoxur, thiofanox, triazamate, trimethacarb, XMC and xylylcarb; or organophosphates, for example acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, famphur, fenitrothion, fosthiazate, heptenophos, imicyafos, isofenphos, isopropyl O-(methoxyaminothiophosphoryl) salicylate, isoxathion, malathion, mecarbam, methidathion, mevinphos, monocrotophos, naled, omethoate, parathion-methyl, phenthoate, phorate, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, and triclorfon.
(2) GABA-gated chloride channel antagonists, such as, for example, cyclodiene-organochlorines, for example chlordane and/or phenylpyrazoles.
(3) Sodium channel modulators/voltage-gated sodium channel blockers such as, for example, pyrethroids, e.g. acrinathrin, allethrin, d-cis-trans allethrin, d-trans allethrin, bifenthrin, bioallethrin, bioallethrin s-cyclopentenyl isomer, bioresmethrin, cycloprothrin, cyhalothrin, lambda-cyhalothrin, gamma-cyhalothrin, empenthrin [(EZ)-(1R)-isomer], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans-isomer], prallethrin, pyrethrins (pyrethrum), resmethrin, tefluthrin, tetramethrin, tetramethrin [(1R)-isomer)], and transfluthrin or DDT or methoxychlor.
(4) Nicotinergic acetylcholine receptor (nAChR) agonists, such as, for example, neonicotinoids, e.g. dinotefuran, nitenpyram, and thiamethoxam or nicotine or sulfoxaflor.
(5) Allosteric activators of the nicotinergic acetylcholine receptor (nAChR) such as, for example, spinosyns, e.g. spinetoram and spinosad.
(6) Chloride channel activators, such as, for example, avermectins/milbemycins, for example abamectin, emamectin benzoate, lepimectin and milbemectin.
(7) Juvenile hormone imitators such as, for example, juvenile hormone analogues, e.g. hydroprene, kinoprene and methoprene or fenoxycarb or pyriproxyfen.
(8) Active compounds with unknown or nonspecific mechanisms of action such as, for example, alkyl halides, e.g. methyl bromide and other alkyl halides; or chloropicrine or sulphuryl fluoride or borax or tartar emetic.
(9) Selective antifeedants, for example pymetrozine or flonicamid.
(10) Mite growth inhibitors, for example clofentezine, hexythiazox and diflovidazin or etoxazole.
(11) Microbial disruptors of the insect gut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins: CrylAb, CrylAc, CrylFa, Cry2Ab, mCry3A, Cry3Ab, Cry3Bb, Cry34/35Ab1.
(12) Oxidative phosphorylation inhibitors, ATP disruptors such as, for example, diafenthiuron or organotin compounds, for example azocyclotin, cyhexatin and fenbutatin oxide or propargite or tetradifon.
(13) Oxidative phosphorylation decouplers acting by interrupting the H proton gradient such as, for example, chlorfenapyr, DNOC and sulfluramid.
(14) Nicotinergic acetylcholine receptor antagonists such as, for example, bensultap, cartap hydrochloride, thiocylam, and thiosultap-sodium.
(15) Chitin biosynthesis inhibitors, type 0, such as, for example, bistrifluron, chlorfluazuron, diflubenzuron, flucycloxuron, flufenoxuron, hexaflumuron, lufenuron, novaluron, noviflumuron, and teflubenzuron.
(16) Chitin biosynthesis inhibitors, type 1, for example buprofezin.
(17) Moulting inhibitors (in particular for Diptera, i.e., dipterans) such as, for example, cyromazine.
(18) Ecdysone receptor agonists such as, for example, chromafenozide, halofenozide, methoxyfenozide and tebufenozide.
(19) Octopaminergic agonists.
(20) Complex-III electron transport inhibitors such as, for example, hydramethylnone or acequinocyl or fluacrypyrim.
(21) Complex-I electron transport inhibitors, for example from the group of the METI acaricides, e.g., fenazaquin, fenpyroximate, pyrimidifen, pyridaben, tebufenpyrad and tolfenpyrad or rotenone (Derris).
(22) Voltage-gated sodium channel blockers, for example indoxacarb or metaflumizone.
(23) Inhibitors of acetyl-CoA carboxylase.
(24) Complex-IV electron transport inhibitors such as, for example, phosphines, e.g. aluminium phosphide, calcium phosphide, phosphine and zinc phosphide or cyanide.
(25) Complex II electron transport inhibitors, such as, for example, cyenopyrafen and cyflumetofen.
(26) Ryanodine receptor effectors, such as, for example, diamides, e.g., chlorantraniliprole, which is also known by the trade name RYNAXYPYR™, and cyantraniliprole; and
further active compounds such as, for example, afidopyropen, azadirachtin, benclothiaz, benzoximate, bifenazate, bromopropylate, chinomethionat, cryolite, dicofol, diflovidazin, fluensulfone, flometoquin, flufenerim, flufenoxystrobin, flufiprole, fufenozide, heptafluthrin, imidaclothiz, meperfluthrin, paichongding, pyflubumide, pyrifluquinazon, pyriminostrobin, tetramethylfluthrin and iodomethane; and also the following compounds: 3-bromo-N-{2-bromo-4-chloro-6-[(1-cyclopropylethyl)carbamoyl]phenyl}-1-(3-chloropyridin-2-yl)-1H-pyrazole-5-carboxamide (known from WO2005/077934), {1′-[(2E)-3-(4-chlorophenyl)prop-2-en-1-yl]-5-fluorospiro[indol-3,4′-piperidin]-1(2H)-yl}(2-chloropyridin-4-yl)methanone (known from WO2003/106457), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494), 3-(2,5-dimethylphenyl)-4-hydroxy-8-methoxy-1,8-diazaspiro[4.5]dec-3-en-2-one (known from WO2009/049851), 3-(2,5-dimethylphenyl)-8-methoxy-2-oxo-1,8-diazaspiro[4.5]dec-3-en-4-yl-ethylcarbonate (known from WO2009/049851), 4-(but-2-yn-1-yloxy)-6-(3,5-dimethylpiperidin-1-yl)-5-fluoropyrimidinc (known from WO2004/099160), 4-(but-2-yn-1-yloxy)-6-(3-chlorophenyl)pyrimidinc (known from WO2003/076415), PF1364 (CAS Reg. No. 1204776-60-2), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO2005/085216), 4-{5-[3-chloro-5-(trifluoromethyl)phenyl]-5-(trifluoromethyl)-4,5-dihydro-1,2-oxazol-3-yl}-N-{2-oxo-2-[(2,2,2-trifluoroethyl)amino]ethyl}-1-naphthamide (known from WO2009/002809), methyl 2-[2-({[3-bromo-1-(3-chloropyridin-2-yl)-1H-pyrazol-5-yl]carbonyl}amino)-5-chloro-3-methylbenzoyl]-2-methylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO2005/085216), methyl 2-[3,5-dibromo-2-({[3-bromo-1-(3-chloropyridin-2-yl)-1H-pyrazol-5-yl]carbonyl}amino)benzoyl]-2-ethylhydrazinecarboxylate (known from WO2005/085216), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN102057925), 3-chloro-N-(2-cyanopropan-2-yl)-N-[4-(1,1,1,2,3,3,3-heptafluoropropan-2-yl)-2-methylphenyl]phthalamide (known from WO2012/034472), 8-chloro-N-[(2-chloro-5-methoxyphenyl)sulphonyl]-6-(trifluoromethyl)imidazo[1,2-a]pyridine-2-carboxamide (known from WO2010/129500), 4-[5-(3,5-dichlorophenyl)-5-(trifluoromethyl)-4,5-dihydro-1,2-oxazol-3-yl]-2-methyl-N-(1-oxidothictan-3-yl)benzamide (known from WO2009/080250), N-[(2E)-1-[(6-chloropyridin-3-yl)methyl]pyridin-2(1H)-ylidene]-2,2,2-trifluoroacetamide (known from WO2012/029672), 1-[(2-chloro-1,3-thiazol-5-yl)methyl]-4-oxo-3-phenyl-4H-pyrido[1,2-a]pyrimidin-1-ium-2-olate (known from WO2009/099929), 1-[(6-chloropyridin-3-yl)methyl]-4-oxo-3-phenyl-4H-pyrido[1,2-a]pyrimidin-1-ium-2-olate (known from WO2009/099929), (5S,8R)-1-[(6-chloropyridin-3-yl)methyl]-9-nitro-2,3,5,6,7,8-hexahydro-1H-5,8-epoxyimidazo[1,2-a]azepine (known from WO2010/069266), (2E)-1-[(6-chloropyridin-3-yl)methyl]-N′-nitro-2-pentylidenchydrazinecarboximidamide (known from WO2010/060231), 4-(3-{2,6-dichloro-4-[(3,3-dichloroprop-2-en-1-yl)oxy]phenoxy}propoxy)-2-methoxy-6-(trifluoromethyl)pyrimidine (known from CN101337940), N-[2-(tert-butylcarbamoyl)-4-chloro-6-methylphenyl]-1-(3-chloropyridin-2-yl)-3-(fluoromethoxy)-1H-pyrazole-5-carboxamide (known from WO2008/134969).

According to a preferred embodiment of the present invention, the at least one insecticide is selected from the group consisting of abamectin, carbofuran, cyazypyr, cycloaprid, chlorantraniliprole, spinosad, sulfoxaflor, tefluthrin, and thiamethoxam.

In a preferred embodiment of the present invention the insecticide is a synthetic insecticide.

### Compositions According to the Present Invention

According to the present invention the composition comprises a) recombinant exosporium-producing Bacillus cells that express a fusion protein comprising: (i) at least one plant growth stimulating protein or peptide selected from the group consisting of an enzyme involved in the production or activation of a plant growth stimulating compound; an enzyme that degrades or modifies a bacterial, fungal, or plant nutrient source; and a protein or peptide that protects a plant from a pathogen; and (ii) a targeting sequence that localizes the fusion protein to the exosporium of the Bacillus cells; and b) at least one particular insecticide disclosed herein in a synergistically effective amount.

A “synergistically effective amount” according to the present invention represents a quantity of a combination of a recombinant exosporium-producing Bacillus cells that express a fusion protein and at least one particular insecticide disclosed herein that is more effective against insects, mites, nematodes and/or phytopathogens than the recombinant exosporium-producing Bacillus cells that express a fusion protein or the insecticide alone. A “synergistically effective amount” according to the present invention also represents a quantity of a combination of a recombinant exosporium-producing Bacillus cells that expresses a fusion protein and at least one particular insecticide disclosed herein that is more effective at enhancing plant growth and/or promoting plant health than the a recombinant exosporium-producing Bacillus cells that express a fusion protein or the insecticide alone.

The present invention comprises each and every combination of each of the particular insecticides disclosed herein with the recombinant exosporium-producing Bacillus cells.

In a highly preferred embodiment the present invention relates to a composition comprising a) recombinant exosporium-producing Bacillus cells that express a fusion protein comprising: (i) at least one plant growth stimulating protein or peptide selected from the group consisting of an enzyme involved in the production or activation of a plant growth stimulating compound; an enzyme that degrades or modifies a bacterial, fungal, or plant nutrient source; and a protein or peptide that protects a plant from a pathogen or pest; and (ii) a targeting sequence that localizes the fusion protein to the exosporium of the Bacillus cells; and b) at least one particular insecticide disclosed herein in a synergistically effective amount and the at least one insecticide is selected from the group consisting of abamectin, carbofuran, cyazypyr, cycloaprid, chlorantraniliprole, spinosad, sulfoxaflor, tefluthrin, and thiamethoxam in a synergistically effective amount.

In a preferred embodiment the composition according to the present invention further comprises at least one fungicide.

In general, “fungicidal” means the ability of a substance to increase mortality or inhibit the growth rate of fungi. The term “fungus” or “fungi” includes a wide variety of nucleated sporebearing organisms that are devoid of chlorophyll. Examples of fungi include yeasts, molds, mildews, rusts, and mushrooms.

### Further Additives

One aspect of the present invention is to provide a composition as described above additionally comprising at least one auxiliary selected from the group consisting of extenders, solvents, spontaneity promoters, carriers, emulsifiers, dispersants, frost protectants, thickeners and adjuvants. Those compositions are referred to as formulations.

Accordingly, in one aspect of the present invention such formulations, and application forms prepared from them, are provided as crop protection agents and/or pesticidal agents, such as drench, drip and spray liquors, comprising the composition of the invention. The application forms may comprise further crop protection agents and/or pesticidal agents, and/or activity-enhancing adjuvants such as penetrants, examples being vegetable oils such as, for example, rapeseed oil, sunflower oil, mineral oils such as, for example, liquid paraffins, alkyl esters of vegetable fatty acids, such as rapeseed oil or soybean oil methyl esters, or alkanol alkoxylates, and/or spreaders such as, for example, alkylsiloxanes and/or salts, examples being organic or inorganic ammonium or phosphonium salts, examples being ammonium sulphate or diammonium hydrogen phosphate, and/or retention promoters such as dioctyl sulphosuccinate or hydroxypropylguar polymers and/or humectants such as glycerol and/or fertilizers such as ammonium, potassium or phosphorous fertilizers, for example.

Examples of typical formulations include water-soluble liquids (SL), emulsifiable concentrates (EC), emulsions in water (EW), suspension concentrates (SC, SE, FS, OD), water-dispersible granules (WG), granules (GR) and capsule concentrates (CS); these and other possible types of formulation are described, for example, by Crop Life International and in Pesticide Specifications, Manual on Development and Use of FAO and WHO Specifications for Pesticides, FAO Plant Production and Protection Papers—173, prepared by the FAO/WHO Joint Meeting on Pesticide Specifications, 2004, ISBN: 9251048576. The formulations may comprise active agrochemical compounds other than one or more active compounds of the invention.

The formulations or application forms in question preferably comprise auxiliaries, such as extenders, solvents, spontaneity promoters, carriers, emulsifiers, dispersants, frost protectants, biocides, thickeners and/or other auxiliaries, such as adjuvants, for example. An adjuvant in this context is a component which enhances the biological effect of the formulation, without the component itself having a biological effect. Examples of adjuvants are agents which promote the retention, spreading, attachment to the leaf surface, or penetration.

These formulations are produced in a known manner, for example by mixing the active compounds with auxiliaries such as, for example, extenders, solvents and/or solid carriers and/or further auxiliaries, such as, for example, surfactants. The formulations are prepared either in suitable plants or else before or during the application.

Suitable for use as auxiliaries are substances which are suitable for imparting to the formulation of the active compound or the application forms prepared from these formulations (such as, e.g., usable crop protection agents, such as spray liquors or seed dressings) particular properties such as certain physical, technical and/or biological properties.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

In principle it is possible to use all suitable solvents. Suitable solvents are, for example, aromatic hydrocarbons, such as xylene, toluene or alkylnaphthalenes, for example, chlorinated aromatic or aliphatic hydrocarbons, such as chlorobenzene, chloroethylene or methylene chloride, for example, aliphatic hydrocarbons, such as cyclohexane, for example, paraffins, petroleum fractions, mineral and vegetable oils, alcohols, such as methanol, ethanol, isopropanol, butanol or glycol, for example, and also their ethers and esters, ketones such as acetone, methyl ethyl ketone, methyl isobutyl ketone or cyclohexanone, for example, strongly polar solvents, such as dimethyl sulphoxide, and water.

All suitable carriers may in principle be used. Suitable carriers are in particular: for example, ammonium salts and ground natural minerals such as kaolins, clays, talc, chalk, quartz, attapulgite, montmorillonite or diatomaceous earth, and ground synthetic minerals, such as finely divided silica, alumina and natural or synthetic silicates, resins, waxes and/or solid fertilizers. Mixtures of such carriers may likewise be used. Carriers suitable for granules include the following: for example, crushed and fractionated natural minerals such as calcite, marble, pumice, sepiolite, dolomite, and also synthetic granules of inorganic and organic meals, and also granules of organic material such as sawdust, paper, coconut shells, maize cobs and tobacco stalks.

Liquefied gaseous extenders or solvents may also be used. Particularly suitable are those extenders or carriers which at standard temperature and under standard pressure are gaseous, examples being aerosol propellants, such as halogenated hydrocarbons, and also butane, propane, nitrogen and carbon dioxide.

Examples of emulsifiers and/or foam-formers, dispersants or wetting agents having ionic or nonionic properties, or mixtures of these surface-active substances, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyltaurates), phosphoric esters of polyethoxylated alcohols or phenols, fatty acid esters of polyols, and derivatives of the compounds containing sulphates, sulphonates and phosphates, examples being alkylaryl polyglycol ethers, alkylsulphonates, alkyl sulphates, arylsulphonates, protein hydrolysates, lignin-sulphite waste liquors and methylcellulose. The presence of a surface-active substance is advantageous if one of the active compounds and/or one of the inert carriers is not soluble in water and if application takes place in water.

Further auxiliaries that may be present in the formulations and in the application forms derived from them include colorants such as inorganic pigments, examples being iron oxide, titanium oxide, Prussian Blue, and organic dyes, such as alizarin dyes, azo dyes and metal phthalocyanine dyes, and nutrients and trace nutrients, such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present. Additionally present may be foam-formers or defoamers.

Furthermore, the formulations and application forms derived from them may also comprise, as additional auxiliaries, stickers such as carboxymethylcellulose, natural and synthetic polymers in powder, granule or latex form, such as gum arabic, polyvinyl alcohol, polyvinyl acetate, and also natural phospholipids, such as cephalins and lecithins, and synthetic phospholipids. Further possible auxiliaries include mineral and vegetable oils.

There may possibly be further auxiliaries present in the formulations and the application forms derived from them. Examples of such additives include fragrances, protective colloids, binders, adhesives, thickeners, thixotropic substances, penetrants, retention promoters, stabilizers, sequestrants, complexing agents, humectants and spreaders. Generally speaking, the active compounds may be combined with any solid or liquid additive commonly used for formulation purposes.

Suitable retention promoters include all those substances which reduce the dynamic surface tension, such as dioctyl sulphosuccinate, or increase the viscoelasticity, such as hydroxypropylguar polymers, for example.

Suitable penetrants in the present context include all those substances which are typically used in order to enhance the penetration of active agrochemical compounds into plants. Penetrants in this context are defined in that, from the (generally aqueous) application liquor and/or from the spray coating, they are able to penetrate the cuticle of the plant and thereby increase the mobility of the active compounds in the cuticle. This property can be determined using the method described in the literature (Baur, et al., 1997, Pesticide Science, 51, 131-152). Examples include alcohol alkoxylates such as coconut fatty ethoxylate (10) or isotridecyl ethoxylate (12), fatty acid esters such as rapeseed or soybean oil methyl esters, fatty amine alkoxylates such as tallowamine ethoxylate (15), or ammonium and/or phosphonium salts such as ammonium sulphate or diammonium hydrogen phosphate, for example.

The formulations preferably comprise between 0.0001% and 98% by weight of active compound or, with particular preference, between 0.01% and 95% by weight of active compound, more preferably between 0.5% and 90% by weight of active compound, based on the weight of the formulation. The content of the active compound is defined as the sum of the recombinant exosporium-producing Bacillus cells and the at least one particular insecticide disclosed herein.

The active compound content of the application forms (crop protection products) prepared from the formulations may vary within wide ranges. The active compound concentration of the application forms may be situated typically between 0.0001% and 95% by weight of active compound, preferably between 0.0001% and 1% by weight, based on the weight of the application form. Application takes place in a customary manner adapted to the application forms.

Furthermore, in one aspect of the present invention a kit of parts is provided comprising a recombinant exosporium-producing Bacillus cells and at least one particular insecticide disclosed herein in a synergistically effective amount in a spatially separated arrangement.

In a further embodiment of the present invention the above-mentioned kit of parts further comprises at least one additional fungicide and/or at least one particular insecticide disclosed herein. The fungicide and/or the insecticide can be present either in the recombinant exosporium-producing Bacillus cells component of the kit of parts or in the insecticide component of the kit of parts being spatially separated or in both of these components. Preferably, the fungicide and/or the insecticide are present in the recombinant exosporium-producing Bacillus cells component.

Moreover, the kit of parts according to the present invention can additionally comprise at least one auxiliary selected from the group consisting of extenders, solvents, spontaneity promoters, carriers, emulsifiers, dispersants, frost protectants, thickeners and adjuvants as mentioned below. This at least one auxiliary can be present either in the recombinant exosporium-producing Bacillus cells component of the kit of parts or in the insecticide component of the kit of parts being spatially separated or in both of these components.

In another aspect of the present invention the composition as described above is used for reducing overall damage of plants and plant parts as well as losses in harvested fruits or vegetables caused by insects, mites, nematodes and/or phytopathogens.

Furthermore, in another aspect of the present invention the composition as described above increases the overall plant health.

The term “plant health” generally comprises various sorts of improvements of plants that are not connected to the control of pests. For example, advantageous properties that may be mentioned are improved crop characteristics including: emergence, crop yields, protein content, oil content, starch content, more developed root system, improved root growth, improved root size maintenance, improved root effectiveness, improved stress tolerance (e.g., against drought, heat, salt, UV, water, cold), reduced ethylene (reduced production and/or inhibition of reception), tillering increase, increase in plant height, bigger leaf blade, less dead basal leaves, stronger tillers, greener leaf color, pigment content, photosynthetic activity, less input needed (such as fertilizers or water), less seeds needed, more productive tillers, earlier flowering, early grain maturity, less plant verse (lodging), increased shoot growth, enhanced plant vigor, increased plant stand and early and better germination.

With regard to the use according to the present invention, improved plant health preferably refers to improved plant characteristics including: crop yield, more developed root system (improved root growth), improved root size maintenance, improved root effectiveness, tillering increase, increase in plant height, bigger leaf blade, less dead basal leaves, stronger tillers, greener leaf color, photosynthetic activity, more productive tillers, enhanced plant vigor, and increased plant stand.

With regard to the present invention, improved plant health preferably especially refers to improved plant properties selected from crop yield, more developed root system, improved root growth, improved root size maintenance, improved root effectiveness, tillering increase, and increase in plant height.

The effect of a composition according to the present invention on plant health as defined herein can be determined by comparing plants which are grown under the same environmental conditions, whereby a part of said plants is treated with a composition according to the present invention and another part of said plants is not treated with a composition according to the present invention. Instead, said other part is not treated at all or treated with a placebo (i.e., an application without a composition according to the invention such as an application without all active ingredients (i.e., without the recombinant exosporium-producing Bacillus cereus family member-based biological control agent as described herein and without an insecticide as described herein), or an application without the recombinant exosporium-producing Bacillus cereus family member-based biological control agent as described herein, or an application without an insecticide as described herein.

The composition according to the present invention may be applied in any desired manner, such as in the form of a seed coating, soil drench, and/or directly in-furrow and/or as a foliar spray and applied either pre-emergence, post-emergence or both. In other words, the composition can be applied to the seed, the plant or to harvested fruits and vegetables or to the soil wherein the plant is growing or wherein it is desired to grow (plant's locus of growth).

Reducing the overall damage of plants and plant parts often results in healthier plants and/or in an increase in plant vigor and yield.

Preferably, the composition according to the present invention is used for treating conventional or transgenic plants or seed thereof.

The present invention also relates to methods for stimulating plant growth using any of the compositions described above comprising recombinant exosporium-producing Bacillus cells that express a fusion protein and at least one particular insecticide disclosed herein. The method for stimulating plant growth comprises applying to a plant, a plant part, to the locus surrounding the plant or in which the plant will be planted (e.g., soil or other growth medium) a composition comprising recombinant exosporium-producing Bacillus cells that express a fusion protein comprising: (i) at least one plant growth stimulating protein or peptide; and (ii) a targeting sequence, exosporium protein, or exosporium protein fragment, and at least one further particular insecticide disclosed herein in a synergistically effective amount.

In another aspect of the present invention a method for reducing overall damage of plants and plant parts as well as losses in harvested fruits or vegetables caused by insects, mites, nematodes and/or phytopathogens is provided comprising the step of simultaneously or sequentially applying the recombinant exosporium-producing Bacillus cells and at least one particular insecticide disclosed herein in a synergistically effective amount.

In another embodiment of the present invention, the composition comprises at least one fungicide and/or at least one insecticide in addition to the recombinant exosporium-producing Bacillus cells and the particular insecticide disclosed herein. In one embodiment, the at least one fungicide is a synthetic fungicide.

The method of the present invention includes the following application methods, namely both of the recombinant exosporium-producing Bacillus cells and the at least one particular insecticide disclosed herein may be formulated into a single, stable composition with an agriculturally acceptable shelf life (so called “solo-formulation”), or being combined before or at the time of use (so called “combined-formulations”).

If not mentioned otherwise, the expression “combination” stands for the various combinations of the recombinant exosporium-producing Bacillus cells and the at least insecticide, and optionally the at least one fungicide, in a solo-formulation, in a single “ready-mix” form, in a combined spray mixture composed from solo-formulations, such as a “tank-mix”, and especially in a combined use of the single active ingredients when applied in a sequential manner, i.e., one after the other within a reasonably short period, such as a few hours or days, e.g., 2 hours to 7 days. The order of applying the composition according to the present invention is not essential for working the present invention. Accordingly, the term “combination” also encompasses the presence of the recombinant exosporium-producing Bacillus cells and the at least one particular insecticide disclosed herein, and optionally the at least one fungicide on or in a plant to be treated or its surrounding, habitat or storage space, e.g., after simultaneously or consecutively applying the recombinant exosporium-producing Bacillus cells and the at least one particular insecticide disclosed herein, and optionally the at least one fungicide to a plant its surrounding, habitat or storage space.

If the recombinant exosporium-producing Bacillus cells and the at least one particular insecticide disclosed herein, and optionally the at least one fungicide are employed or used in a sequential manner, it is preferred to treat the plants or plant parts (which includes seeds and plants emerging from the seed), harvested fruits and vegetables according to the following method: Firstly applying the at least one particular insecticide disclosed herein and optionally the at least one fungicide and/or the at least one additional insecticide on the plant or plant parts, and secondly applying the recombinant exosporium-producing Bacillus cells to the same plant or plant parts. By this application manner the amount of residues of insecticides/fungicides on the plant upon harvesting is as low as possible. The time periods between the first and the second application within a (crop) growing cycle may vary and depend on the effect to be achieved. For example, the first application is done to prevent an infestation of the plant or plant parts with insects, mites, nematodes and/or phytopathogens (this is particularly the case when treating seeds) or to combat the infestation with insects, mites, nematodes and/or phytopathogens (this is particularly the case when treating plants and plant parts) and the second application is done to prevent or control the infestation with insects, mites, nematodes and/or phytopathogens and/or to promote plant growth. Control in this context means that the recombinant exosporium-producing Bacillus cells are not able to fully exterminate the pests or phytopathogenic fungi but are able to keep the infestation on an acceptable level.

The present invention also provides methods of enhancing the killing, inhibiting, preventative and/or repelling activity of the compositions of the present invention by multiple applications. In some other embodiments, the compositions of the present invention are applied to a plant and/or plant part for two times, during any desired development stages or under any predetermined pest pressure, at an interval of about 1 hour, about 5 hours, about 10 hours, about 24 hours, about two days, about 3 days, about 4 days, about 5 days, about 1 week, about 10 days, about two weeks, about three weeks, about 1 month or more. Still in some embodiments, the compositions of the present invention are applied to a plant and/or plant part for more than two times, for example, 3 times, 4 times, 5 times, 6 times, 7 times, 8 times, 9 times, 10 times, or more, during any desired development stages or under any predetermined pest pressure, at an interval of about 1 hour, about 5 hours, about 10 hours, about 24 hours, about two days, about 3 days, about 4 days, about 5 days, about 1 week, about 10 days, about two weeks, about three weeks, about 1 month or more. The intervals between each application can vary if it is desired. One skilled in the art will be able to determine the application times and length of interval depending on plant species, plant pest species, and other factors.

By following the before mentioned steps, a very low level of residues of the at least one fungicide and/or at least one particular insecticide disclosed herein and/or additional insecticide on the treated plant, plant parts, and the harvested fruits and vegetables can be achieved.

If not mentioned otherwise the treatment of plants or plant parts (which includes seeds and plants emerging from the seed), harvested fruits and vegetables with the composition according to the invention is carried out directly or by action on their surroundings, habitat or storage space using customary treatment methods, for example dipping, spraying, atomizing, irrigating, evaporating, dusting, fogging, broadcasting, foaming, painting, spreading-on, watering (drenching), drip irrigating. It is furthermore possible to apply the recombinant exosporium-producing Bacillus cells, the at least one particular insecticide disclosed herein, and optionally the at least one fungicide as solo-formulation or combined-formulations by the ultra-low volume method, or to inject the composition according to the present invention as a composition or as sole-formulations into the soil (in-furrow).

The term “plant to be treated” encompasses every part of a plant including its root system and the material—e.g., soil or nutrition medium—which is in a radius of at least 10 cm, 20 cm, 30 cm around the caulis or bole of a plant to be treated or which is at least 10 cm, 20 cm, 30 cm around the root system of said plant to be treated, respectively.

The amount of the recombinant exosporium-producing Bacillus cells which is used or employed in combination with at least one particular insecticide disclosed herein, optionally in the presence of at least one fungicide, depends on the final formulation as well as size or type of the plant, plant parts, seeds, harvested fruits and vegetables to be treated. Usually, the recombinant exosporium-producing Bacillus cells to be employed or used according to the invention is present in about 1% to about 80% (w/w), preferably in about 1% to about 60% (w/w), more preferably about 10% to about 50% (w/w) of its solo-formulation or combined-formulation with the at least one particular insecticide disclosed herein, and optionally the fungicide.

Also the amount of the at least one particular insecticide disclosed herein which is used or employed in combination with the recombinant exosporium-producing Bacillus cells, optionally in the presence of at least one fungicide, depends on the final formulation as well as size or type of the plant, plant parts, seeds, harvested fruit or vegetable to be treated. Usually, the recombinant exosporium-producing Bacillus cells to be employed or used according to the invention is present in about 0.1% to about 80% (w/w), preferably 1% to about 60% (w/w), more preferably about 10% to about 50% (w/w) of its solo-formulation or combined-formulation with the at least one particular insecticide disclosed herein, and optionally the at least one fungicide.

Application of the recombinant exosporium-producing Bacillus cells may be effected as a foliar spray, as a soil treatment, and/or as a seed treatment/dressing. When used as a foliar treatment, in one embodiment, about 1/16 to about 5 gallons of whole broth are applied per acre. When used as a soil treatment, in one embodiment, about 1 to about 5 gallons of whole broth are applied per acre. When used for seed treatment about 1/32 to about ¼ gallons of whole broth are applied per acre. For seed treatment, the end-use formulation contains 1×10⁴, at least 1×10⁵, at least 1×10⁶, 1×10⁷, at least 1×10⁸, at least 1×10⁹, or at least 1×10¹⁰ colony forming units per gram.

The recombinant exosporium-producing Bacillus cells and at least one particular insecticide disclosed herein, and if present preferably also the fungicide are used or employed in a synergistic weight ratio. The skilled person is able to find out the synergistic weight ratios for the present invention by routine methods. The skilled person understands that these ratios refer to the ratio within a combined-formulation as well as to the calculative ratio of the recombinant exosporium-producing Bacillus cells described herein and the at least one particular insecticide disclosed herein when both components are applied as mono-formulations to a plant to be treated. The skilled person can calculate this ratio by simple mathematics since the volume and the amount of the recombinant exosporium-producing Bacillus cells and the at least one particular insecticide disclosed herein, respectively, in a mono-formulation is known to the skilled person.

The ratio can be calculated based on the amount of the at least one particular insecticide disclosed herein, at the time point of applying said component of a combination according to the invention to a plant or plant part and the amount of recombinant exosporium-producing Bacillus cells shortly prior (e.g., 48 h, 24 h, 12 h, 6 h, 2 h, 1 h) or at the time point of applying said component of a combination according to the invention to a plant or plant part.

The application of the recombinant exosporium-producing Bacillus cells and the at least one particular insecticide disclosed herein to a plant or a plant part can take place simultaneously or at different times as long as both components are present on or in the plant after the application(s). In cases where the recombinant exosporium-producing Bacillus cells and insecticide are applied at different times and insecticide is applied noticeable prior to the recombinant exosporium-producing Bacillus cells, the skilled person can determine the concentration of insecticide on/in a plant by chemical analysis known in the art, at the time point or shortly before the time point of applying the recombinant exosporium-producing Bacillus cells. Vice versa, when the recombinant exosporium-producing Bacillus cells are applied to a plant first, the concentration of the recombinant exosporium-producing Bacillus cells agent can be determined using tests which are also known in the art, at the time point or shortly before the time point of applying the insecticide.

In particular, in one embodiment the synergistic weight ratio of the recombinant exosporium-producing Bacillus cells and the at least one particular insecticide disclosed herein lies in the range of 1:1000 to 1000:1, preferably in the range of 1:500 to 500:1, more preferably in the range of 1:300 to 500:1. Especially preferred ratios are between 20:1 and 1:20, such as 10:1, 5:1 or 2:1. It has to be noted that these ratio ranges refer to the recombinant Bacillus cereus family member-based biological control agent (to be combined with at least one particular insecticide or a preparation of at least one particular insecticide disclosed herein). For example, a ratio of 100:1 means 100 weight parts of a spore preparation of the recombinant exosporium-producing Bacillus-based biological control agent and 1 weight part of insecticide are combined (either as a solo formulation, a combined formulation or by separate applications to plants so that the combination is formed on the plant). In one aspect of this embodiment, the spore preparation of the recombinant exosporium-producing Bacillus cells is a dried spore preparation containing at least about 1×10⁴ cfu/g, at least about 1×10⁵ cfu/g, at least about 1×10⁶ cfu/g at least about 1×10⁷ cfu/g, at least about 1×10⁸ cfu/g, at least about 1×10⁹ cfu/g, at least about 1×10¹⁰ cfu/g, or at least about 1×10¹¹ cfu/g.

In another embodiment, the synergistic weight ratio of the recombinant exosporium-producing Bacillus cells and the at least one particular insecticide disclosed herein is in the range of 1:100 to 20,000:1, preferably in the range of 1:50 to 10,000:1 or even in the range of 1:50 to 1000:1.

In one embodiment of the present invention, the concentration of the recombinant exosporium-producing Bacillus cells after dispersal is at least 50 g/ha, such as 50-7500 g/ha, 50-2500 g/ha, 50-1500 g/ha; at least 250 g/ha (hectare), at least 500 g/ha or at least 800 g/ha.

The application rate of composition to be employed or used according to the present invention may vary. The skilled person is able to find the appropriate application rate by way of routine experiments.

In another aspect of the present invention a seed treated with the composition as described above is provided.

The control of insects, mites, nematodes and/or phytopathogens by treating the seed of plants has been known for a long time and is a subject of continual improvements. Nevertheless, the treatment of seed entails a series of problems which cannot always be solved in a satisfactory manner. Thus, it is desirable to develop methods for protecting the seed and the germinating plant that remove the need for, or at least significantly reduce, the additional delivery of crop protection compositions in the course of storage, after sowing or after the emergence of the plants. It is desirable, furthermore, to optimize the amount of active ingredient employed in such a way as to provide the best-possible protection to the seed and the germinating plant from attack by insects, mites, nematodes and/or phytopathogens, but without causing damage to the plant itself by the active ingredient employed. In particular, methods for treating seed ought also to take into consideration the intrinsic insecticidal and/or nematicidal properties of pest-resistant or pest-tolerant transgenic plants, in order to achieve optimum protection of the seed and of the germinating plant with a minimal use of crop protection compositions.

The present invention therefore also relates in particular to a method for protecting seed and germinating plants from attack by pests, by treating the seed with the recombinant exosporium-producing Bacillus cells as defined above and at least one particular insecticide disclosed herein in a synergistically effective amount. The method of the invention for protecting seed and germinating plants from attack by pests encompasses a method in which the seed is treated simultaneously in one operation with the recombinant exosporium-producing Bacillus cells agent and the at least one particular insecticide disclosed herein, and optionally the at least one fungicide. It also encompasses a method in which the seed is treated at different times with the recombinant exosporium-producing Bacillus cells and the at least one particular insecticide disclosed herein, and optionally the at least one fungicide.

The invention likewise relates to the use of the composition of the invention for treating seed for the purpose of protecting the seed and the resultant plant against insects, mites, nematodes and/or phytopathogens.

The invention also relates to seed which at the same time has been treated with a recombinant exosporium-producing Bacillus cells and at least one particular insecticide disclosed herein, and optionally at least one fungicide. The invention further relates to seed which has been treated at different times with the recombinant exosporium-producing Bacillus cells and the at least one particular insecticide disclosed herein and optionally the at least one fungicide and/or the at least one insecticide. In the case of seed which has been treated at different times with the recombinant exosporium-producing Bacillus cells and the at least one particular insecticide disclosed herein, and optionally the at least one fungicide, the individual active ingredients in the composition of the invention may be present in different layers on the seed.

Furthermore, the invention relates to seed which, following treatment with the composition of the invention, is subjected to a film-coating process in order to prevent dust abrasion of the seed.

One of the advantages of the present invention is that, owing to the particular systemic properties of the compositions of the invention, the treatment of the seed with these compositions provides protection from insects, mites, nematodes and/or phytopathogens not only to the seed itself but also to the plants originating from the seed, after they have emerged. In this way, it may not be necessary to treat the crop directly at the time of sowing or shortly thereafter.

A further advantage is to be seen in the fact that, through the treatment of the seed with composition of the invention, germination and emergence of the treated seed may be promoted.

It is likewise considered to be advantageous composition of the invention may also be used, in particular, on transgenic seed.

It is also stated that the composition of the invention may be used in combination with agents of the signalling technology, as a result of which, for example, colonization with symbionts is improved, such as rhizobia, mycorrhiza and/or endophytic bacteria, for example, is enhanced, and/or nitrogen fixation is optimized.

The compositions of the invention are suitable for protecting seed of any variety of plant which is used in agriculture, in greenhouses, in forestry or in horticulture. More particularly, the seed in question is that of cereals (e.g., wheat, barley, rye, oats and millet), maize, cotton, soybeans, rice, potatoes, sunflower, coffee, tobacco, canola, oilseed rape, beets (e.g., sugar beet and fodder beet), peanuts, vegetables (e.g., tomato, cucumber, bean, brassicas, onions and lettuce), fruit plants, lawns and ornamentals. Particularly important is the treatment of the seed of cereals (such as wheat, barley, rye and oats) maize, soybeans, cotton, canola, oilseed rape and rice.

As already mentioned above, the treatment of transgenic seed with the composition of the invention is particularly important. The seed in question here is that of plants which generally contain at least one heterologous gene that controls the expression of a polypeptide having, in particular, insecticidal and/or nematicidal properties. These heterologous genes in transgenic seed may come from microorganisms such as Bacillus, Rhizobium, Pseudomonas, Serratia, Trichoderma, Clavibacter, Glomus or Gliocladium. The present invention is particularly suitable for the treatment of transgenic seed which contains at least one heterologous gene from Bacillus sp. With particular preference, the heterologous gene in question comes from Bacillus thuringiensis.

For the purposes of the present invention, the composition of the invention is applied alone or in a suitable formulation to the seed. The seed is preferably treated in a condition in which its stability is such that no damage occurs in the course of the treatment. Generally speaking, the seed may be treated at any point in time between harvesting and sowing. Typically, seed is used which has been separated from the plant and has had cobs, hulls, stems, husks, hair or pulp removed. Thus, for example, seed may be used that has been harvested, cleaned and dried to a moisture content of less than 15% by weight. Alternatively, seed can also be used that after drying has been treated with water, for example, and then dried again.

When treating seed it is necessary, generally speaking, to ensure that the amount of the composition of the invention, and/or of other additives, that is applied to the seed is selected such that the germination of the seed is not adversely affected, and/or that the plant which emerges from the seed is not damaged. This is the case in particular with active ingredients which may exhibit phytotoxic effects at certain application rates.

The compositions of the invention can be applied directly, in other words without comprising further components and without having been diluted. As a general rule, it is preferable to apply the compositions in the form of a suitable formulation to the seed. Suitable formulations and methods for seed treatment are known to the skilled person and are described in, for example, the following documents: U.S. Pat. Nos. 4,272,417 A; 4,245,432 A; 4,808,430 A; 5,876,739 A; U.S. Patent Publication No. 2003/0176428 A1; WO 2002/080675 A1, WO 2002/028186 A2.

The combinations which can be used in accordance with the invention may be converted into the customary seed-dressing formulations, such as solutions, emulsions, suspensions, powders, foams, slurries or other coating compositions for seed, and also ULV formulations.

These formulations are prepared in a known manner, by mixing composition with customary adjuvants, such as, for example, customary extenders and also solvents or diluents, colorants, wetters, dispersants, emulsifiers, antifoams, preservatives, secondary thickeners, stickers, gibberellins, and also water.

Colorants which may be present in the seed-dressing formulations which can be used in accordance with the invention include all colorants which are customary for such purposes. In this context it is possible to use not only pigments, which are of low solubility in water, but also water-soluble dyes. Examples include the colorants known under the designations Rhodamin B, C.I. Pigment Red 112 and C.I. Solvent Red 1.

Wetters which may be present in the seed-dressing formulations which can be used in accordance with the invention include all of the substances which promote wetting and which are customary in the formulation of active agrochemical ingredients. Use may be made preferably of alkylnaphthalenesulphonates, such as diisopropyl- or diisobutyl-naphthalenesulphonates.

Dispersants and/or emulsifiers which may be present in the seed-dressing formulations which can be used in accordance with the invention include all of the nonionic, anionic and cationic dispersants that are customary in the formulation of active agrochemical ingredients. Use may be made preferably of nonionic or anionic dispersants or of mixtures of nonionic or anionic dispersants. Suitable nonionic dispersants are, in particular, ethylene oxide-propylene oxide block polymers, alkylphenol polyglycol ethers and also tristryrylphenol polyglycol ethers, and the phosphated or sulphated derivatives of these. Suitable anionic dispersants are, in particular, lignosulphonates, salts of polyacrylic acid, and arylsulphonate-formaldehyde condensates.

Antifoams which may be present in the seed-dressing formulations which can be used in accordance with the invention include all of the foam inhibitors that are customary in the formulation of active agrochemical ingredients. Use may be made preferably of silicone antifoams and magnesium stearate.

Preservatives which may be present in the seed-dressing formulations which can be used in accordance with the invention include all of the substances which can be employed for such purposes in agrochemical compositions. Examples include dichlorophen and benzyl alcohol hemiformal.

Secondary thickeners which may be present in the seed-dressing formulations which can be used in accordance with the invention include all substances which can be used for such purposes in agrochemical compositions. Those contemplated with preference include cellulose derivatives, acrylic acid derivatives, xanthan, modified clays and highly disperse silica.

Stickers which may be present in the seed-dressing formulations which can be used in accordance with the invention include all customary binders which can be used in seed-dressing products. Preferred mention may be made of polyvinylpyrrolidone, polyvinyl acetate, polyvinyl alcohol and tylose.

Gibberellins which may be present in the seed-dressing formulations which can be used in accordance with the invention include preferably the gibberellins A1, A3 (=gibberellic acid), A4 and A7, with gibberellic acid being used with particular preference. The gibberellins are known (cf. R. Wegler, “Chemie der Pflanzenschutz-und Schädlingsbekämpfungsmittel”, Volume 2, Springer Verlag, 1970, pp. 401-412).

The seed-dressing formulations which can be used in accordance with the invention may be used, either directly or after prior dilution with water, to treat seed of any of a wide variety of types. Accordingly, the concentrates or the preparations obtainable from them by dilution with water may be employed to dress the seed of cereals, such as wheat, barley, rye, oats and triticale, and also the seed of maize, rice, oilseed rape, peas, beans, cotton, sunflowers and beets, or else the seed of any of a very wide variety of vegetables. The seed-dressing formulations which can be used in accordance with the invention, or their diluted preparations, may also be used to dress seed of transgenic plants. In that case, additional synergistic effects may occur in interaction with the substances formed through expression.

For the treatment of seed with the seed-dressing formulations which can be used in accordance with the invention, or with the preparations produced from them by addition of water, suitable mixing equipment includes all such equipment which can typically be employed for seed dressing. More particularly, the procedure when carrying out seed dressing is to place the seed in a mixer, to add the particular desired amount of seed-dressing formulations, either as such or following dilution with water beforehand, and to carry out mixing until the distribution of the formulation on the seed is uniform. This may be followed by a drying operation.

The application rate of the seed-dressing formulations which can be used in accordance with the invention may be varied within a relatively wide range. It is guided by the particular amount of the recombinant exosporium-producing Bacillus cereus family member-based biological control agent and the at least one particular insecticide disclosed herein in the formulations, and by the seed. The application rates in the case of the composition are situated generally at between 0.001 and 50 g per kilogram of seed, preferably between 0.01 and 15 g per kilogram of seed.

The compositions according to the invention, in case they exhibit insecticidal and miticidal and/or nematicidal activity, in combination with good plant tolerance and favourable toxicity to warm-blooded animals and being tolerated well by the environment, are suitable for protecting plants and plant organs, for increasing harvest yields, for improving the quality of the harvested material and for controlling animal pests, in particular insects, mites, arachnids, helminths, nematodes and molluscs, which are encountered in agriculture, in horticulture, in animal husbandry, in forests, in gardens and leisure facilities, in protection of stored products and of materials, and in the hygiene sector. They can be preferably employed as plant protection agents. In particular, the present invention relates to the use of the composition according to the invention as insecticide and/or fungicide.

They are active against normally sensitive and resistant species and against all or some stages of development. The abovementioned pests include:
pests from the phylum Arthropoda, especially from the class Arachnida, for example, Acarus spp., Aceria sheldoni, Aculops spp., Aculus spp., Amblyomma spp., Amphitetranychus viennensis, Argas spp., Boophilus spp., Brevipalpus spp., Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., Epitrimerus pyri, Eutetranychus spp., Eriophyes spp., Glycyphagus domesticus, Halotydeus destructor, Hemitarsonemus spp., Hyalomma spp., Ixodes spp., Latrodectus spp., Loxosceles spp., Metatetranychus spp., Neutrombicula autumnalis, Nuphersa spp., Oligonychus spp., Ornithodorus spp., Ornithonyssus spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., Tetranychus spp., Trombicula alfreddugesi, Vacjovis spp., Vasates lycopersici;
in particular clover mite, brown mite, hazelnut spider mite, asparagus spider mite, brown wheat mite, legume mite, oxalis mite, boxwood mite, Texas citrus mite, Oriental red mite, citrus red mite, European red mite, yellow spider mite, fig spider mite, Lewis spider mite, six-spotted spider mite, Willamette mite Yuma spider mite, web-spinning mite, pineapple mite, citrus green mite, honey-locust spider mite, tea red spider mite, southern red mite, avocado brown mite, spruce spider mite, avocado red mite, Banks grass mite, carmine spider mite, desert spider mite, vegetable spider mite, tumid spider mite, strawberry spider mite, two-spotted spider mite, McDaniel mite, Pacific spider mite, hawthorn spider mite, four-spotted spider mite, Schoenei spider mite, Chilean false spider mite, citrus flat mite, privet mite, flat scarlet mite, white-tailed mite, pineapple tarsonemid mite, West Indian sugar cane mite, bulb scale mite, cyclamen mite, broad mite, winter grain mite, red-legged earth mite, filbert big-bud mite, grape erineum mite, pear blister leaf mite, apple leaf edgeroller mite, peach mosaic vector mite, alder bead gall mite, Perian walnut leaf gall mite, pecan leaf edgeroll mite, fig bud mite, olive bud mite, citrus bud mite, litchi erineum mite, wheat curl mite, coconut flower and nut mite, sugar cane blister mite, buffalo grass mite, bermuda grass mite, carrot bud mite, sweet potato leaf gall mite, pomegranate leaf curl mite, ash sprangle gall mite, maple bladder gall mite, alder erineum mite, redberry mite, cotton blister mite, blueberry bud mite, pink tea rust mite, ribbed tea mite, grey citrus mite, sweet potato rust mite, horse chestnut rust mite, citrus rust mite, apple rust mite, grape rust mite, pear rust mite, flat needle sheath pine mite, wild rose bud and fruit mite, dryberry mite, mango rust mite, azalea rust mite, plum rust mite, peach silver mite, apple rust mite, tomato russet mite, pink citrus rust mite, cereal rust mite, rice rust mite;
from the class Chilopoda, for example, Geophilus spp., Scutigera spp.;
from the order or the class Collembola, for example, Onychiurus armatus;
from the class Diplopoda, for example, Blaniulus guttulatus;
from the class Insecta, e.g. from the order Blattodea, for example, Blattella asahinai, Blattella germanica, Blatta orientalis, Leucophaca maderae, Panchlora spp., Parcoblatta spp., Periplaneta spp., Supella longipalpa;
from the order Coleoptera, for example, Acalymma vittatum, Acanthoscelides obtectus, Adoretus spp., Agelastica alni, Agriotes spp., Alphitobius diaperinus, Amphimallon solstitialis, Anobium punctatum, Anoplophora spp., Anthonomus spp., Anthrenus spp., Apion spp., Apogonia spp., Atomaria spp., Attagenus spp., Bruchidius obtectus, Bruchus spp., Cassida spp., Cerotoma trifurcata, Ceutorrhynchus spp., Chactocnema spp., Clconus mendicus, Conoderus spp., Cosmopolites spp., Costelytra zealandica, Ctenicera spp., Curculio spp., Cryptolestes ferrugineus, Cryptorhynchus lapathi, Cylindrocopturus spp., Dermestes spp., Diabrotica spp., Dichocrocis spp., Dicladispa armigera, Diloboderus spp., Epilachna spp., Epitrix spp., Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., Lachnosterna consanguinea, Lasioderma serricorne, Latheticus oryzae, Lathridius spp., Lema spp., Leptinotarsa decemlineata, Leucoptera spp., Lissorhoptrus oryzophilus, Lixus spp., Luperodes spp., Lyctus spp., Megascelis spp., Melanotus spp., Meligethes aeneus, Melolontha spp., Migdolus spp., Monochamus spp., Naupactus xanthographus, Necrobia spp., Niptus hololeucus, Oryctes rhinoceros, Oryzaephilus surinamensis, Oryzaphagus oryzae, Otiorrhynchus spp., Oxycetonia jucunda, Phaedon cochleariae, Phyllophaga spp., Phyllophaga helleri, Phyllotreta spp., Popillia japonica, Premnotrypes spp., Prostephanus truncatus, Psylliodes spp., Ptinus spp., Rhizobius ventralis, Rhizopertha dominica, Sitophilus spp., Sitophilus oryzac, Sphenophorus spp., Stegobium paniceum, Sternechus spp., Symphyletes spp., Tanymecus spp., Tenebrio molitor, Tenebrioides mauretanicus, Tribolium spp., Trogoderma spp., Tychius spp., Xylotrechus spp., Zabrus spp.;
preferably from Banded cucumber beetle (Diabrotica balteata), Northern corn rootworm (Diabrotica barberi), Southern corn rootworm (Diabrotica undecimpunctata howardi), Western cucumber beetle (Diabrotica undecimpunctata tenella), Western spotted cucumber beetle (Diabrotica undecimpunctata undecimpunctata), Western corn rootworm (Diabrotica virgifera virgifera), Mexican corn rootworm (Diabrotica virgifera zeae);
from the order Diptera, for example, Aedes spp., Agromyza spp., Anastrepha spp., Anopheles spp., Asphondylia spp., Bactrocera spp., Bibio hortulanus, Calliphora erythrocephala, Calliphora vicina, Ceratitis capitata, Chironomus spp., Chrysomyia spp., Chrysops spp., Chrysozona pluvialis, Cochliomyia spp., Contarinia spp., Cordylobia anthropophaga, Cricotopus sylvestris, Culex spp., Culicoides spp., Culiseta spp., Cuterebra spp., Dacus oleae, Dasyneura spp., Delia spp., Dermatobia hominis, Drosophila spp., Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., Lucilia spp., Lutzomyia spp., Mansonia spp., Musca spp., Oestrus spp., Oscinella frit, Paratanytarsus spp., Paralauterborniella subcincta, Pegomyia spp., Phlebotomus spp., Phorbia spp., Phormia spp., Piophila casci, Prodiplosis spp., Psila rosae, Rhagoletis spp., Sarcophaga spp., Simulium spp., Stomoxys spp., Tabanus spp., Tetanops spp., Tipula spp.;
from the order Heteroptera, for example, Anasa tristis, Antestiopsis spp., Boisea spp., Blissus spp., Calocoris spp., Campylomma livida, Cavelerius spp., Cimex spp., Collaria spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptocorisa varicornis, Leptoglossus phyllopus, Lygus spp., Macropes excavatus, Miridae, Monalonion atratum, Nezara spp., Ocbalus spp., Pentomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., Triatoma spp.;
from the order Homoptera, for example, Acizzia acaciaebaileyanae, Acizzia dodonaeae, Acizzia uncatoides, Acrida turrita, Acyrthosipon spp., Acrogonia spp., Aeneolamia spp., Agonoscena spp., Aleyrodes proletella, Aleurolobus barodensis, Aleurothrixus floccosus, Allocaridara malayensis, Amrasca spp., Anuraphis cardui, Aonidiella spp., Aphanostigma piri, Aphis spp., Arboridia apicalis, Arytainilla spp., Aspidiella spp., Aspidiotus spp., Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., Calligypona marginata, Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., Cryptomyzus ribis, Cryptoneossa spp., Ctenarytaina spp., Dalbulus spp., Dialeurodes citri, Diaphorina citri, Diaspis spp., Drosicha spp., Dysaphis spp., Dysmicoccus spp., Empoasca spp., Eriosoma spp., Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Macrosteles facifrons, Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nettigoniclla spectra, Nilaparvata lugens, Oncometopia spp., Orthezia praelonga, Oxya chinensis, Pachypsylla spp., Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Prosopidopsylla flava, Protopulvinaria pyriformis, Pseudaulacaspis pentagona, Pseudococcus spp., Psyllopsis spp., Psylla spp., Pteromalus spp., Pyrilla spp., Quadraspidiotus spp., Quesada gigas, Rastrococcus spp., Rhopalosiphum spp., Saissetia spp., Scaphoideus titanus, Schizaphis graminum, Selenaspidus articulatus, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., Trialeurodes vaporariorum, Trioza spp., Typhlocyba spp., Unaspis spp., Viteus vitifolii, Zygina spp.;
from the order Hymenoptera, for example, Acromyrmex spp., Athalia spp., Atta spp., Diprion spp., Hoplocampa spp., Lasius spp., Monomorium pharaonis, Sirex spp., Solenopsis invicta, Tapinoma spp., Urocerus spp., Vespa spp., Xeris spp.;
from the order Isopoda, for example, Armadillidium vulgare, Oniscus asellus, Porcellio scaber;
from the order Isoptera, for example, Coptotermes spp., Cornitermes cumulans, Cryptotermes spp., Incisitermes spp., Microtermes obesi, Odontotermes spp., Reticulitermes spp.;
from the order Lepidoptera, for example, Achroia grisella, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharina, Ephestia spp., Epinotia spp., Epiphyas postvittana, Etiella spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamstra brassicae, Melanitis leda, Mocis spp., Monopis obviella, Mythimna separata, Nemapogon cloacellus, Nymphula spp., Oiketicus spp., Oria spp., Orthaga spp., Ostrinia spp., Oulema oryzae, Panolis flammea, Parnara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella, Prays spp., Prodenia spp., Protoparce spp., Pseudaletia spp., Pseudaletia unipuncta, Pseudoplusia includens, Pyrausta nubilalis, Rachiplusia nu, Schoenobius spp., Scirpophaga spp., Scirpophaga innotata, Scotia segetum, Sesamia spp., Sesamia inferens, Sparganothis spp., Spodoptera spp., Spodoptera praefica, Stathmopoda spp., Stomopteryx subsecivella, Synanthedon spp., Tecia solanivora, Thermesia gemmatalis, Tinca cloacella, Tinea pellionella, Tincola bisselliella, Tortrix spp., Trichophaga tapetzella, Trichoplusia spp., Tryporyza incertulas, Tuta absoluta, Virachola spp.;
from the order Orthoptera or Saltatoria, for example, Acheta domesticus, Dichroplus spp., Gryllotalpa spp., Hieroglyphus spp., Locusta spp., Melanoplus spp., Schistocerca gregaria;
from the order Phthiraptera, for example, Damalinia spp., Haematopinus spp., Linognathus spp., Pediculus spp., Ptirus pubis, Trichodectes spp.;
from the order Psocoptera for example Lepinatus spp., Liposcelis spp.;
from the order Siphonaptera, for example, Ceratophyllus spp., Ctenocephalides spp., Pulex irritans, Tunga penetrans, Xenopsylla cheopsis;
from the order Thysanoptera, for example, Anaphothrips obscurus, Baliothrips biformis, Drepanothrips reuteri, Enneothrips flavens, Frankliniella spp., Heliothrips spp., Hercinothrips femoralis, Rhipiphorothrips cruentatus, Scirtothrips spp., Taeniothrips cardamomi, Thrips spp.;
from the order Zygentoma (=Thysanura), for example, Ctenolepisma spp., Lepisma saccharina, Lepismodes inquilinus, Thermobia domestica;
from the class Symphyla, for example, Scutigerella spp.;
pests from the phylum Mollusca, especially from the class Bivalvia, for example, Dreissena spp., and from the class Gastropoda, for example, Arion spp., Biomphalaria spp., Bulinus spp., Deroceras spp., Galba spp., Lymnaea spp., Oncomelania spp., Pomacea spp., Succinea spp.;
animal pests from the phylums Plathelminthes and Nematoda, for example, Ancylostoma duodenale, Ancylostoma ceylanicum, Acylostoma braziliensis, Ancylostoma spp., Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichiura, Wuchereria bancrofti;
phytoparasitic pests from the phylum Nematoda, for example, Aphelenchoides spp., Bursaphelenchus spp., Ditylenchus spp., Globodera spp., Heterodera spp., Longidorus spp., Meloidogyne spp., Pratylenchus spp., Radopholus spp., Trichodorus spp., Tylenchulus spp., Xiphinema spp., Helicotylenchus spp., Tylenchorhynchus spp., Scutellonema spp., Paratrichodorus spp., Meloinema spp., Paraphelenchus spp., Aglenchus spp., Belonolaimus spp., Nacobbus spp., Rotylenchulus spp., Rotylenchus spp., Neotylenchus spp., Paraphelenchus spp., Dolichodorus spp., Hoplolaimus spp., Punctodera spp., Criconemella spp., Quinisulcius spp., Hemicycliophora spp., Anguina spp., Subanguina spp., Hemicriconemoides spp., Psilenchus spp., Pseudohalenchus spp., Criconemoides spp., Cacopaurus spp., Hirschmaniella spp, Tetylenchus spp.

The fact that the composition is well tolerated by plants at the concentrations required for controlling plant diseases and pests allows the treatment of above-ground parts of plants, of propagation stock and seeds, and of the soil.

According to the invention all plants and plant parts can be treated. By plants is meant all plants and plant populations such as desirable and undesirable wild plants, cultivars and plant varieties (whether or not protectable by plant variety or plant breeder's rights). Cultivars and plant varieties can be plants obtained by conventional propagation and breeding methods which can be assisted or supplemented by one or more biotechnological methods such as by use of double haploids, protoplast fusion, random and directed mutagenesis, molecular or genetic markers or by bioengineering and genetic engineering methods. By plant parts is meant all above ground and below ground parts and organs of plants such as shoot, leaf, blossom and root, whereby for example leaves, needles, stems, branches, blossoms, fruiting bodies, fruits and seed as well as roots, corms and rhizomes are listed. Crops and vegetative and generative propagating material, for example cuttings, corms, rhizomes, runners and seeds also belong to plant parts.

The inventive composition, when it is well tolerated by plants, has favourable homeotherm toxicity and is well tolerated by the environment, is suitable for protecting plants and plant organs, for enhancing harvest yields, for improving the quality of the harvested material. It can preferably be used as crop protection composition. It is active against normally sensitive and resistant species and against all or some stages of development.

Plants which can be treated in accordance with the invention include the following main crop plants: maize, soya bean, alfalfa, cotton, sunflower, Brassica oil seeds such as Brassica napus (e.g. canola, rapeseed), Brassica rapa, B. juncea (e.g. (field) mustard) and Brassica carinata, Arecaceae sp. (e.g. oilpalm, coconut), rice, wheat, sugar beet, sugar cane, oats, rye, barley, millet and sorghum, triticale, flax, nuts, grapes and vine and various fruit and vegetables from various botanic taxa, e.g. Rosaceae sp. (e.g. pome fruits such as apples and pears, but also stone fruits such as apricots, cherries, almonds, plums and peaches, and berry fruits such as strawberries, raspberries, red and black currant and gooseberry), Ribesioidae sp., Juglandaceae sp., Betulaceae sp., Anacardiaceae sp., Fagaceae sp., Moraceae sp., Oleaceae sp. (e.g. olive tree), Actinidaceae sp., Lauraceae sp. (e.g. avocado, cinnamon, camphor), Musaceae sp. (e.g. banana trees and plantations), Rubiaceae sp. (e.g. coffee), Theaceae sp. (e.g. tea), Sterculiceae sp., Rutaceae sp. (e.g. lemons, oranges, mandarins and grapefruit); Solanaceae sp. (e.g. tomatoes, potatoes, peppers, capsicum, aubergines, tobacco), Liliaceae sp., Compositae sp. (e.g. lettuce, artichokes and chicory—including root chicory, endive or common chicory), Umbelliferae sp. (e.g. carrots, parsley, celery and celeriac), Cucurbitaceae sp. (e.g. cucumbers—including gherkins, pumpkins, watermelons, calabashes and melons), Alliaceae sp. (e.g. leeks and onions), Cruciferae sp. (e.g. white cabbage, red cabbage, broccoli, cauliflower, Brussels sprouts, pak choi, kohlrabi, radishes, horseradish, cress and chinese cabbage), Leguminosas sp. (e.g. peanuts, peas, lentils and beans—e.g. common beans and broad beans), Chenopodiaceae sp. (e.g. Swiss chard, fodder beet, spinach, beetroot), Linaceae sp. (e.g. hemp), Cannabeacea sp. (e.g. cannabis), Malvaceae sp. (e.g. okra, cocoa), Papaveraceae (e.g. poppy), Asparagaceae (e.g. asparagus); useful plants and ornamental plants in the garden and woods including turf, lawn, grass and Stevia rebaudiana; and in each case genetically modified types of these plants.

Depending on the plant species or plant cultivars, their location and growth conditions (soils, climate, vegetation period, diet), using or employing the composition according to the present invention the treatment according to the invention may also result in super-additive (“synergistic”) effects. Thus, for example, by using or employing inventive composition in the treatment according to the invention, reduced application rates and/or a widening of the activity spectrum and/or an increase in the activity better plant growth, increased tolerance to high or low temperatures, increased tolerance to drought or to water or soil salt content, increased flowering performance, easier harvesting, accelerated maturation, higher harvest yields, bigger fruits, larger plant height, greener leaf color, earlier flowering, higher quality and/or a higher nutritional value of the harvested products, higher sugar concentration within the fruits, better storage stability and/or processability of the harvested products are possible, which exceed the effects which were actually to be expected.

At certain application rates of the inventive composition in the treatment according to the invention may also have a strengthening effect in plants. The defense system of the plant against attack by unwanted phytopathogenic fungi and/or microorganisms and/or viruses is mobilized. Plant-strengthening (resistance-inducing) substances are to be understood as meaning, in the present context, those substances or combinations of substances which are capable of stimulating the defense system of plants in such a way that, when subsequently inoculated with unwanted phytopathogenic fungi and/or microorganisms and/or viruses, the treated plants display a substantial degree of resistance to these phytopathogenic fungi and/or microorganisms and/or viruses. Thus, by using or employing composition according to the present invention in the treatment according to the invention, plants can be protected against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are also preferably to be treated according to the invention are resistant against one or more biotic stresses, i.e., said plants show a better defense against animal and microbial pests, such as against nematodes, insects, mites, phytopathogenic fungi, bacteria, viruses and/or viroids.

Plants and plant cultivars which may also be treated according to the invention are those plants which are resistant to one or more abiotic stresses, i.e., that already exhibit an increased plant health with respect to stress tolerance. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance. Preferably, the treatment of these plants and cultivars with the composition of the present invention additionally increases the overall plant health (cf. above).

Plants and plant cultivars which may also be treated according to the invention, are those plants characterized by enhanced yield characteristics, i.e., that already exhibit an increased plant health with respect to this feature. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation.

Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or car number, seed number per pod or car, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability. Preferably, the treatment of these plants and cultivars with the composition of the present invention additionally increases the overall plant health (cf. above).

Plants that may be treated according to the invention are hybrid plants that already express the characteristic of heterosis or hybrid vigor which results in generally higher yield, vigor, health and resistance towards biotic and abiotic stress factors. Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling, i.e., the mechanical removal of the male reproductive organs (or males flowers) but, more typically, male sterility is the result of genetic determinants in the plant genome. In that case, and especially when seed is the desired product to be harvested from the hybrid plants it is typically useful to ensure that male fertility in the hybrid plants is fully restored. This can be accomplished by ensuring that the male parents have appropriate fertility restorer genes which are capable of restoring the male fertility in hybrid plants that contain the genetic determinants responsible for male-sterility. Genetic determinants for male sterility may be located in the cytoplasm. Examples of cytoplasmic male sterility (CMS) were for instance described in Brassica species. However, genetic determinants for male sterility can also be located in the nuclear genome. Male sterile plants can also be obtained by plant biotechnology methods such as genetic engineering. A particularly useful means of obtaining male-sterile plants is described in WO 89/10396 in which, for example, a ribonuclease such as barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e., plants made tolerant to one or more given herbicides. Such plants can be obtained either by genetic transformation, or by selection of plants containing a mutation imparting such herbicide tolerance.

### EXAMPLES

### Example 1: Formula for the Efficacy of the Combination of Multiple Active Ingredients

A synergistic effect of active ingredients is present when the activity of the active ingredient combinations exceeds the total of the activities of the active ingredients when applied individually. The expected activity for a given combination of two active ingredients can be calculated as follows (cf. Colby, S. R., “Calculating Synergistic and Antagonistic Responses of Herbicide Combinations,” Weeds, 1967, 15, 20-22):

If
X is the efficacy when active ingredient A is applied at an application rate of m ppm (or g/ha),
Y is the efficacy when active ingredient B is applied at an application rate of n ppm (or g/ha),
E is the efficacy when the active ingredients A and B are applied at application rates of m and n ppm (or g/ha), respectively, and
then

$$E = X + Y - \frac{X \cdot Y}{100}$$

If the actual activity exceeds the calculated value, then the activity of the combination is superadditive, i.e., a synergistic effect exists. In this case, the efficacy which was actually observed must be greater than the value for the expected efficacy (E) calculated from the above-mentioned formula.

For instance, the formula and analysis can be applied to an evaluation of plant growth promotion. Such an assay is evaluated several days after the applications to plants. 100% means plant weight which corresponds to that of the untreated control plant. Efficacy means in this case the additional % of plant weight in comparison to that of the untreated control. For example, a treatment that resulted in plant weights that were 120% compared to the untreated control plant would have an efficacy of 20%. If the plant growth promotion effect for the combination (i.e., the observed efficacy for % plant weights of plants treated with the combination) exceeds the calculated value, then the activity of the combination is superadditive, i.e., a synergistic effect exists.

The formula and analysis can also be used to evaluate synergy in disease control assays. The degree of efficacy expressed in % is denoted. 0% means an efficacy which corresponds to that of the control while an efficacy of 100% means that no disease is observed.

If the actual insecticidal or fungicidal activity exceeds the calculated value, then the activity of the combination is superadditive, i.e., a synergistic effect exists. In this case, the efficacy which is actually observed must be greater than the value for the expected efficacy (E) calculated from the above-mentioned formula.

A further way of demonstrating a synergistic effect is the method of Tammes (cf. “Isoboles, A Graphic Representation of Synergism in Pesticides,” in Neth. J. Plant Path., 1964, 70, 73-80).

### Example 2. Plant Growth Promotion with Abamectin and Recombinant Bacillus thuringiensis Cells

Maize seeds will be grown in loamy sand in the greenhouse at 20° C. and 70% humidity for about 11 days. After about 11 days from the time of treatment the seedlings will be cut off above the soil and the fresh weight will be determined.

Recombinant Bacillus thuringiensis cells expressing an endoglucanase encoded by SEQ ID NO: 107 will be applied at about 50 μg/kernel. A recombinant Bacillus cereus family member (Bacillus thuringiensis BT013A) expressing endoglucanase on its exosporium (BEE) may be generated as follows. To generate plasmids for expression of fusion proteins in Bacillus cereus family members, PCR fragments are generated that encode the BclA promoter (SEQ ID NO: 85), a methionine start codon, and amino acids 20-35 of BclA (SEQ ID NO: 1) followed by a six alanine linker sequence fused in frame to Bacillus thuringiensis BT013A endoglucanase (SEQ ID NO: 108). These PCR fragments are digested with Xhol and ligated into the SalI site of the pSUPER plasmid to generate the plasmids pSUPER-BclA 20-35-Endoglucanase. The pSUPER plasmid is generated through fusion of the pUC57 plasmid (containing an ampicillin resistance cassette) with the pBC16-1 plasmid from Bacillus (containing a tetracycline resistance). This 5.5 kbp plasmid can replicate in both E. coli and Bacillus spp. The pSUPER-BclA 20-35-Endoglucanase plasmids are transformed into and propagated in dam methylase negative E. coli strains and finally are transformed into Bacillus thuringiensis BT013A.

To obtain whole broth cultures of BEE, 15 mL conicals containing brain heart infusion media (BHI) are inoculated with BEE and grown for 7-8 hours at around 30° C. at a shaker setting of 300 rpm. The next day, 250 μL aliquots from each flask are inoculated into 250 mL flasks containing 50 mL of a yeast extract-based media and grown at about 30° C. After approximately 2 days of incubation, when sporulation is at least 95% completed, the culture broth is harvested and colony forming units calculated. The fermentation broth is diluted to 5% in 50 mL water and the following colony forming units may be applied to each seed. Abamectin will also be applied at about 250 μg/kernel.

It is expected that the maize plants treated with the recombinant Bacillus thuringiensis in combination with the abamectin will have % shoot weights that exceed the calculated value based on the % shoot weights from the maize plants treated with the two active ingredients alone, i.e., a synergistic effect will be observed.

### Example 3. Plant Growth Promotion with Spinosad and Recombinant Bacillus thuringiensis Cells

Maize seeds will be grown in loamy sand in the greenhouse at 20° C. and 70% humidity for about 11 days. After about 11 days from the time of treatment the seedlings will be cut off above the soil and the fresh weight will be determined.

Recombinant Bacillus thuringiensis cells expressing an endoglucanase encoded by SEQ ID NO: 107 will be applied at about 50 μg/kernel, as described above. Spinosad will also be applied at about 250 μg/kernel.

It is expected that the maize plants treated with the recombinant Bacillus thuringiensis in combination with the spinosad will have % shoot weights that exceed the calculated value based on the % shoot weights from the maize plants treated with the two active ingredients alone, i.e., a synergistic effect will be observed.

The above experiments may also be conducted with recombinant Bacillus thuringiensis cells expressing a phospholipase having SEQ ID NO: 108, and a synergistic effect is expected.

1-25. (canceled)

26. A method of treating a plant, a plant part, or the locus surrounding the plant to enhance plant growth and/or promote plant health comprising the step of simultaneously or sequentially applying:
a) recombinant exosporium-producing Bacillus cells of a Bacillus cereus family member that express a fusion protein comprising:
(i) an endoglucanase having at least 95% sequence identity to SEQ ID NO: 107; and
(ii) a targeting sequence, exosporium protein, or exosporium protein fragment; and
b) abamectin;
in a synergistically effective amount.

27. The method according to claim 26, wherein the targeting sequence or exosporium protein comprises:
an amino acid sequence having at least about 43% identity with amino acids 20-35 of SEQ ID NO: 1, wherein the identity with amino acids 25-35 is at least about 54%;
a targeting sequence comprising amino acids 1-35 of SEQ ID NO: 1;
a targeting sequence comprising amino acids 20-35 of SEQ ID NO: 1;
a targeting sequence comprising amino acids 22-31 of SEQ ID NO: 1;
a targeting sequence comprising amino acids 22-33 of SEQ ID NO: 1;
a targeting sequence comprising amino acids 20-31 of SEQ ID NO: 1;
a targeting sequence comprising SEQ ID NO: 1; or
an exosporium protein comprising an amino acid sequence having at least 85% identity with SEQ ID NO: 2.

28-31. (canceled)

32. The method according of claim 26, wherein the Bacillus cereus family member is selected from the group consisting of Bacillus anthracis, Bacillus cereus, Bacillus thuringiensis, Bacillus mycoides, Bacillus pseudomycoides, Bacillus samanii, Bacillus gaemokensis, Bacillus weihenstephensis, Bacillus toyoiensis and combinations thereof.

33. The method of claim 26, wherein the fusion protein comprises SEQ ID NO: 107.

34. The method according to claim 26, wherein the fusion protein is expressed under the control of a sporulation promoter native to the targeting sequence of the fusion protein.

35. The method according to claim 26, wherein the fusion protein is expressed under the control of a high-expression sporulation promoter.

36. The method according to claim 26, wherein the sporulation promoter comprises a nucleic acid sequence having at least 95% identity with a nucleic acid sequence of any one of SEQ ID NOs: 85-103.

37. The method according to claim 26, wherein the method comprises applying the recombinant exosporium-producing Bacillus cells and abamectin to a plant seed.

38. The method according to claim 26, wherein the method comprises applying the recombinant exosporium-producing Bacillus cells and abamectin to the plant.

39. The method according to claim 26, wherein the method comprises applying the recombinant exosporium-producing Bacillus cells and abamectin to the locus surrounding the plant.

40. The method according to claim 26, wherein the method comprises applying the recombinant exosporium-producing Bacillus cells and abamectin simultaneously.

41. The method according to claim 26, wherein the method comprises applying the recombinant exosporium-producing Bacillus cells and abamectin sequentially.

42. The method according to claim 26, wherein the targeting sequence comprises an amino acid sequence having at least about 43% identity with amino acids 20-35 of SEQ ID NO: 1, wherein the identity with amino acids 25-35 is at least about 54%; and the recombinant Bacillus cells are derived from Bacillus thuringiensis or Bacillus mycoides.

43. The method according to claim 42, wherein the fusion protein comprises SEQ ID NO: 107: the targeting sequence comprises amino acids 20-35 of SEQ ID NO: 1; and the Bacillus thuringiensis is Bacillus thuringiensis BT013A.
